SEQUENCE LISTING (28 sequences)

SEQ ID NO: 1 - protein, 133 aa, Homo sapiens [sequence omitted]

SEQ ID NO: 2 - DNA, 507 bp, Artificial Sequence
  Synthetic Construct
atgtacagcatgcagctggccagctgcgtgacactgaccctcgtgctgctggtgaacagcgctcctacctcctccagcaccagcagcagcaccgctgaggcccagcagcagcagcagcaacagcaacagcagcaacaacatttagaacagctgctgatggatttacaagaactgctgtctcgtatggagaactatcgtaatttaaagctgcctcgtatgctgaccgccaagttcgctttacccaagcaagctacagagctgaaggatttacagtgtttagaggacgagctgggccctctgaggcatgtgctggacggcacccagagcaagagcttccagctggaggacgccgagaactttatcagcaacattcgtgtgaccgtggtgaagctgaagggcagcgacaacaccttcgagtgccagttcgacgacgagagcgccacagtggtggactttttaagaaggtggatcgccttctgccagtccatcatcagcaccagcccccag

SEQ ID NO: 3 - protein, 169 aa, Artificial Sequence
  Synthetic Construct
MYSMQLASCVTLTLVLLVNSAPTSSSTSSSTAEAQQQQQQQQQQQQHLEQLLMDLQELLSRMENYRNLKLPRMLTAKFALPKQATELKDLQCLEDELGPLRHVLDGTQSKSFQLEDAENFISNIRVTVVKLKGSDNTFECQFDDESATVVDFLRRWIAFCQSIISTSPQ

SEQ ID NO: 4 - DNA, 1960 bp, Artificial Sequence
  Synthetic Construct
gattacgacgtgctaatctagcgtgtgaagacgataaattaatgatctatggattaccatggatgacaactcaaacatctgcgttatcaataaatagtaaaccgatagtgtataaagattgtgcaaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactctcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctattagatacatagatcctcgtcgcgatatcgcattttctaacgtgatggatatattaaagtcgaataaagtgaacaataattaattctttattgtcatcatgaacgggcgcgcctataaaaattgaaattttattttttttttttggaatataaatatccctatcagtgatagagatctccctatcagtgatagagagccaccatgtacagcatgcagctggccagctgcgtgacactgaccctcgtgctgctggtgaacagcgctcctacctcctccagcaccagcagcagcaccgctgaggcccagcagcagcagcagcaacagcaacagcagcaacaacatttagaacagctgctgatggatttacaagaactgctgtctcgtatggagaactatcgtaatttaaagctgcctcgtatgctgaccgccaagttcgctttacccaagcaagctacagagctgaaggatttacagtgtttagaggacgagctgggccctctgaggcatgtgctggacggcacccagagcaagagcttccagctggaggacgccgagaactttatcagcaacattcgtgtgaccgtggtgaagctgaagggcagcgacaacaccttcgagtgccagttcgacgacgagagcgccacagtggtggactttttaagaaggtggatcgccttctgccagtccatcatcagcaccagcccccagtaatgagcgatcgcgtgtagaaagtgttacatcgactcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatcggctcctggtacacatatacgttatttgagagatcatttctataatttaggaatgattatcaaatggatgctaattgacggacgccatcatgatcctattctaaatggattgcgtgatgtgactctagtgactcggttcgttgatgaggaatatctacgatccatcaaaaaacaactgcatccttctaagattattttaatttctgatgtaagatccaaacgaggaggaaatgaacctagtacggcggatttactaagtaattacgctctacaaaatgtcatgattagtattttaaaccccgtggcatctagtcttaaatggagatgcccgtttccagatcaatggatcaaggacttttatatcccacacggtaataaaatgttacaaccttttgctccttcatattcagctgaa

SEQ ID NO: 5 - DNA, 1954 bp, Artificial Sequence
  Synthetic Construct
gattacgacgtgctaatctagcgtgtgaagacgataaattaatgatctatggattaccatggatgacaactcaaacatctgcgttatcaataaatagtaaaccgatagtgtataaagattgtgcaaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactttcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctgttagatacatagatcctcgtcgcaatatcgcattttctaacgtgatggatatattaaagtcgaataaagtgaacaataattaattctttattgtcatcatgaacgtataaaaattgaaattttattttttttttttggaatataaatatccctatcagtgatagagatctccctatcagtgatagagagccaccatgtacagcatgcagctggccagctgcgtgacactgaccctcgtgctgctggtgaacagcgctcctacctcctccagcaccagcagcagcaccgctgaggcccagcagcagcagcagcaacagcaacagcagcaacaacatttagaacagctgctgatggatttacaagaactgctgtctcgtatggagaactatcgtaatttaaagctgcctcgtatgctgaccgccaagttcgctttacccaagcaagctacagagctgaaggatttacagtgtttagaggacgagctgggccctctgaggcatgtgctggacggcacccagagcaagagcttccagctggaggacgccgagaactttatcagcaacattcgtgtgaccgtggtgaagctgaagggcagcgacaacaccttcgagtgccagttcgacgacgagagcgccacagtggtggactttttaagaaggtggatcgccttctgccagtccatcatcagcaccagcccccagtaatgagcgatcgcgtgtagaaagtgttacatcgactcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatctgctcccggtacacatatacgttatttgagagatcatttctataatttaggagtgatcatcaaatggatgctaattgacggccgccatcatgatcctattttaaatggattgcgtgatgtgactctagtgactcggttcgttgatgaggaatatctacgatccatcaaaaaacaactgcatccttctaagattattttaatttctgatgtgagatccaaacgaggaggaaatgaacctagtacggcggatttactaagtaattacgctctacaaaatgtcatgattagtattttaaaccccgtggcgtctagtcttaaatggagatgcccgtttccagatcaatggatcaaggacttttatatcccacacggtaataaaatgttacaaccttttgctccttcatattcagctgaaat

SEQ ID NO: 6 - DNA, 5869 bp, Artificial Sequence
  Synthetic Construct
tgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacacggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcactgttaaaaggacaattacaaacaggaatcaaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacaggtcgacgaattcgattacgacgtgctaatctagcgtgtgaagacgataaattaatgatctatggattaccatggatgacaactcaaacatctgcgttatcaataaatagtaaaccgatagtgtataaagattgtgcaaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactctcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctattagatacatagatcctcgtcgcgatatcgcattttctaacgtgatggatatattaaagtcgaataaagtgaacaataattaattctttattgtcatcatgaacgggcgcgcctataaaaattgaaattttattttttttttttggaatataaatatccctatcagtgatagagatctccctatcagtgatagagagccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgggatcccagaccctgaactttgatctgctgaaactggcaggcgatgtggaaagcaacccaggcccaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgagcgatcgcgtgtagaaagtgttacatcgactcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatcggctcctggtacacatatacgttatttgagagatcatttctataatttaggaatgattatcaaatggatgctaattgacggacgccatcatgatcctattctaaatggattgcgtgatgtgactctagtgactcggttcgttgatgaggaatatctacgatccatcaaaaaacaactgcatccttctaagattattttaatttctgatgtaagatccaaacgaggaggaaatgaacctagtacggcggatttactaagtaattacgctctacaaaatgtcatgattagtattttaaaccccgtggcatctagtcttaaatggagatgcccgtttccagatcaatggatcaaggacttttatatcccacacggtaataaaatgttacaaccttttgctccttcatattcagctgaaatgaattc

SEQ ID NO: 7 - DNA, 3943 bp, Artificial Sequence
  Synthetic Construct
tgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacacggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcactgttaaaaggacaattacaaacaggaatcaaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacaggtcgacgaattcgattacgacgtgctaatctagcgtgtgaagacgataaattaatgatctatggattaccatggatgacaactcaaacatctgcgttatcaataaatagtaaaccgatagtgtataaagattgtgcaaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactctcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctattagatacatagatcctcgtcgcgatatcgcattttctaacgtgatggatatattaaagtcgaataaagtgaacaataattaattctttattgtcatcatgaacgggcgcgcctataaaaattgaaattttattttttttttttggaatataaatatccctatcagtgatagagatctccctatcagtgatagagagccaccatgtacagcatgcagctggccagctgcgtgacactgaccctcgtgctgctggtgaacagcgctcctacctcctccagcaccagcagcagcaccgctgaggcccagcagcagcagcagcaacagcaacagcagcaacaacatttagaacagctgctgatggatttacaagaactgctgtctcgtatggagaactatcgtaatttaaagctgcctcgtatgctgaccgccaagttcgctttacccaagcaagctacagagctgaaggatttacagtgtttagaggacgagctgggccctctgaggcatgtgctggacggcacccagagcaagagcttccagctggaggacgccgagaactttatcagcaacattcgtgtgaccgtggtgaagctgaagggcagcgacaacaccttcgagtgccagttcgacgacgagagcgccacagtggtggactttttaagaaggtggatcgccttctgccagtccatcatcagcaccagcccccagtaatgagcgatcgcgtgtagaaagtgttacatcgactcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatcggctcctggtacacatatacgttatttgagagatcatttctataatttaggaatgattatcaaatggatgctaattgacggacgccatcatgatcctattctaaatggattgcgtgatgtgactctagtgactcggttcgttgatgaggaatatctacgatccatcaaaaaacaactgcatccttctaagattattttaatttctgatgtaagatccaaacgaggaggaaatgaacctagtacggcggatttactaagtaattacgctctacaaaatgtcatgattagtattttaaaccccgtggcatctagtcttaaatggagatgcccgtttccagatcaatggatcaaggacttttatatcccacacggtaataaaatgttacaaccttttgctccttcatattcagctgaaatgaattc

SEQ ID NO: 8 - DNA, 3935 bp, Artificial Sequence
  Synthetic Construct
tgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacacggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcactgttaaaaggacaattacaaacaggaatcaaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacaggtcgacgaattcgattacgacgtgctaatctagcgtgtgaagacgataaattaatgatctatggattaccatggatgacaactcaaacatctgcgttatcaataaatagtaaaccgatagtgtataaagattgtgcaaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactttcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctgttagatacatagatcctcgtcgcaatatcgcattttctaacgtgatggatatattaaagtcgaataaagtgaacaataattaattctttattgtcatcatgaacgtataaaaattgaaattttattttttttttttggaatataaatatccctatcagtgatagagatctccctatcagtgatagagagccaccatgtacagcatgcagctggccagctgcgtgacactgaccctcgtgctgctggtgaacagcgctcctacctcctccagcaccagcagcagcaccgctgaggcccagcagcagcagcagcaacagcaacagcagcaacaacatttagaacagctgctgatggatttacaagaactgctgtctcgtatggagaactatcgtaatttaaagctgcctcgtatgctgaccgccaagttcgctttacccaagcaagctacagagctgaaggatttacagtgtttagaggacgagctgggccctctgaggcatgtgctggacggcacccagagcaagagcttccagctggaggacgccgagaactttatcagcaacattcgtgtgaccgtggtgaagctgaagggcagcgacaacaccttcgagtgccagttcgacgacgagagcgccacagtggtggactttttaagaaggtggatcgccttctgccagtccatcatcagcaccagcccccagtaatgagcgatcgcgtgtagaaagtgttacatcgactcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatctgctcccggtacacatatacgttatttgagagatcatttctataatttaggagtgatcatcaaatggatgctaattgacggccgccatcatgatcctattttaaatggattgcgtgatgtgactctagtgactcggttcgttgatgaggaatatctacgatccatcaaaaaacaactgcatccttctaagattattttaatttctgatgtgagatccaaacgaggaggaaatgaacctagtacggcggatttactaagtaattacgctctacaaaatgtcatgattagtattttaaaccccgtggcgtctagtcttaaatggagatgcccgtttccagatcaatggatcaaggacttttatatcccacacggtaataaaatgttacaaccttttgctccttcatattcagctgaaatgaattc

SEQ ID NO: 9 - protein, 133 aa, Artificial Sequence
  Synthetic Construct
APTSSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFCQSIISTLT

SEQ ID NO: 10 - DNA, 399 bp, Artificial Sequence
  Synthetic Construct
gcccctaccagctcctccaccaagaagacccagctgcagctggagcatttactgctggatttacagatgattttaaacggcatcaacaactacaagaaccccaagctgactcgtatgctgaccgccaagttcgctatgcccaagaaggccaccgagctgaagcacctccagtgtttagaggaggagctgaagcctttagaggaggtgctgaatggagcccagagcaagaatttccatttaaggcctcgtgatttaatcagcaacatcaacgtgatcgtgctggagctgaaaggctccgagaccaccttcatgtgcgagtacgccgacgagaccgccaccatcgtggagtttttaaatcgttggatcaccttctgccagagcatcatcagcactttaacc

SEQ ID NO: 11 - DNA, 399 bp, Artificial Sequence
  Synthetic Construct
gcgccaacatcaagttcgaccaagaagacgcagttgcagctagagcatttgcttttggatcttcaaatgatccttaatggtataaataattataagaaccccaaattgacgcgaatgctaacagctaaattcgcaatgccaaagaaggcaaccgagttaaagcacctacaatgcttggaagaagaactaaaaccccttgaggaggtattaaatggtgctcagtcgaagaattttcatcttcgacctcgagacctaatttcaaatattaacgtaattgttttggaattaaagggttcggaaactacttttatgtgtgagtacgcagacgagacagctacaatagtggagtttcttaaccgttggataaccttttgtcaatcaatcatttcgactttgacc

SEQ ID NO: 12 - DNA, 459 bp, Artificial Sequence
  Synthetic Construct
atgtatcgtatgcagctgctgagctgcatcgctttatctttagctttagtgaccaacagcgcccctaccagctcctccaccaagaagacccagctgcagctggagcatttactgctggatttacagatgattttaaacggcatcaacaactacaagaaccccaagctgactcgtatgctgaccgccaagttcgctatgcccaagaaggccaccgagctgaagcacctccagtgtttagaggaggagctgaagcctttagaggaggtgctgaatggagcccagagcaagaatttccatttaaggcctcgtgatttaatcagcaacatcaacgtgatcgtgctggagctgaaaggctccgagaccaccttcatgtgcgagtacgccgacgagaccgccaccatcgtggagtttttaaatcgttggatcaccttctgccagagcatcatcagcactttaacc

SEQ ID NO: 13 - DNA, 459 bp, Artificial Sequence
  Synthetic Construct
atgtatcgaatgcaattactttcctgtatcgcactttcattagcccttgtgaccaactcagcgccaacatcaagttcgaccaagaagacgcagttgcagctagagcatttgcttttggatcttcaaatgatccttaatggtataaataattataagaaccccaaattgacgcgaatgctaacagctaaattcgcaatgccaaagaaggcaaccgagttaaagcacctacaatgcttggaagaagaactaaaaccccttgaggaggtattaaatggtgctcagtcgaagaattttcatcttcgacctcgagacctaatttcaaatattaacgtaattgttttggaattaaagggttcggaaactacttttatgtgtgagtacgcagacgagacagctacaatagtggagtttcttaaccgttggataaccttttgtcaatcaatcatttcgactttgacc

SEQ ID NO: 14 - protein, 153 aa, Artificial Sequence
  Synthetic Construct
MYRMQLLSCIALSLALVTNSAPTSSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFCQSIISTLT

SEQ ID NO: 15 - DNA, 1914 bp, Artificial Sequence
  Synthetic Construct
gattacgacgtgctaatctagcgtgtgaagacgataaattaatgatctatggattaccatggatgacaactcaaacatctgcgttatcaataaatagtaaaccgatagtgtataaagattgtgcaaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactctcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctattagatacatagatcctcgtcgcgatatcgcattttctaacgtgatggatatattaaagtcgaataaagtgaacaataattaattctttattgtcatcatgaacgggcgcgcctataaaaattgaaattttattttttttttttggaatataaatatccctatcagtgatagagatctccctatcagtgatagagagccaccatgtatcgtatgcagctgctgagctgcatcgctttatctttagctttagtgaccaacagcgcccctaccagctcctccaccaagaagacccagctgcagctggagcatttactgctggatttacagatgattttaaacggcatcaacaactacaagaaccccaagctgactcgtatgctgaccgccaagttcgctatgcccaagaaggccaccgagctgaagcacctccagtgtttagaggaggagctgaagcctttagaggaggtgctgaatggagcccagagcaagaatttccatttaaggcctcgtgatttaatcagcaacatcaacgtgatcgtgctggagctgaaaggctccgagaccaccttcatgtgcgagtacgccgacgagaccgccaccatcgtggagtttttaaatcgttggatcaccttctgccagagcatcatcagcactttaacctaatgagcgatcgcgtgtagaaagtgttacatcgactcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatcggctcctggtacacatatacgttatttgagagatcatttctataatttaggaatgattatcaaatggatgctaattgacggacgccatcatgatcctattctaaatggattgcgtgatgtgactctagtgactcggttcgttgatgaggaatatctacgatccatcaaaaaacaactgcatccttctaagattattttaatttctgatgtaagatccaaacgaggaggaaatgaacctagtacggcggatttactaagtaattacgctctacaaaatgtcatgattagtattttaaaccccgtggcatctagtcttaaatggagatgcccgtttccagatcaatggatcaaggacttttatatcccacacggtaataaaatgttacaaccttttgctccttcatattcagctgaaat

SEQ ID NO: 16 - DNA, 3895 bp, Artificial Sequence
  Synthetic Construct
tgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacacggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcactgttaaaaggacaattacaaacaggaatcaaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacaggtcgacgaattcgattacgacgtgctaatctagcgtgtgaagacgataaattaatgatctatggattaccatggatgacaactcaaacatctgcgttatcaataaatagtaaaccgatagtgtataaagattgtgcaaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactctcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctattagatacatagatcctcgtcgcgatatcgcattttctaacgtgatggatatattaaagtcgaataaagtgaacaataattaattctttattgtcatcatgaacgggcgcgcctataaaaattgaaattttattttttttttttggaatataaatatccctatcagtgatagagatctccctatcagtgatagagagccaccatgtatcgtatgcagctgctgagctgcatcgctttatctttagctttagtgaccaacagcgcccctaccagctcctccaccaagaagacccagctgcagctggagcatttactgctggatttacagatgattttaaacggcatcaacaactacaagaaccccaagctgactcgtatgctgaccgccaagttcgctatgcccaagaaggccaccgagctgaagcacctccagtgtttagaggaggagctgaagcctttagaggaggtgctgaatggagcccagagcaagaatttccatttaaggcctcgtgatttaatcagcaacatcaacgtgatcgtgctggagctgaaaggctccgagaccaccttcatgtgcgagtacgccgacgagaccgccaccatcgtggagtttttaaatcgttggatcaccttctgccagagcatcatcagcactttaacctaatgagcgatcgcgtgtagaaagtgttacatcgactcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatcggctcctggtacacatatacgttatttgagagatcatttctataatttaggaatgattatcaaatggatgctaattgacggacgccatcatgatcctattctaaatggattgcgtgatgtgactctagtgactcggttcgttgatgaggaatatctacgatccatcaaaaaacaactgcatccttctaagattattttaatttctgatgtaagatccaaacgaggaggaaatgaacctagtacggcggatttactaagtaattacgctctacaaaatgtcatgattagtattttaaaccccgtggcatctagtcttaaatggagatgcccgtttccagatcaatggatcaaggacttttatatcccacacggtaataaaatgttacaaccttttgctccttcatattcagctgaaatgaattc

SEQ ID NO: 17 - DNA, 1914 bp, Artificial Sequence
  Synthetic Construct
gattacgacgtgctaatctagcgtgtgaagacgataaattaatgatctatggattaccatggatgacaactcaaacatctgcgttatcaataaatagtaaaccgatagtgtataaagattgtgcaaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactctcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctattagatacatagatcctcgtcgcgatatcgcattttctaacgtgatggatatattaaagtcgaataaagtgaacaataattaattctttattgtcatcatgaacgggcgcgcctataaaaattgaaattttattttttttttttggaatataaatatccctatcagtgatagagatctccctatcagtgatagagagccaccatgtatcgaatgcaattactttcctgtatcgcactttcattagcccttgtgaccaactcagcgccaacatcaagttcgaccaagaagacgcagttgcagctagagcatttgcttttggatcttcaaatgatccttaatggtataaataattataagaaccccaaattgacgcgaatgctaacagctaaattcgcaatgccaaagaaggcaaccgagttaaagcacctacaatgcttggaagaagaactaaaaccccttgaggaggtattaaatggtgctcagtcgaagaattttcatcttcgacctcgagacctaatttcaaatattaacgtaattgttttggaattaaagggttcggaaactacttttatgtgtgagtacgcagacgagacagctacaatagtggagtttcttaaccgttggataaccttttgtcaatcaatcatttcgactttgacctaatgagcgatcgcgtgtagaaagtgttacatcgactcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatcggctcctggtacacatatacgttatttgagagatcatttctataatttaggaatgattatcaaatggatgctaattgacggacgccatcatgatcctattctaaatggattgcgtgatgtgactctagtgactcggttcgttgatgaggaatatctacgatccatcaaaaaacaactgcatccttctaagattattttaatttctgatgtaagatccaaacgaggaggaaatgaacctagtacggcggatttactaagtaattacgctctacaaaatgtcatgattagtattttaaaccccgtggcatctagtcttaaatggagatgcccgtttccagatcaatggatcaaggacttttatatcccacacggtaataaaatgttacaaccttttgctccttcatattcagctgaaat

SEQ ID NO: 18 - DNA, 3895 bp, Artificial Sequence
  Synthetic Construct
tgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacacggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcactgttaaaaggacaattacaaacaggaatcaaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacaggtcgacgaattcgattacgacgtgctaatctagcgtgtgaagacgataaattaatgatctatggattaccatggatgacaactcaaacatctgcgttatcaataaatagtaaaccgatagtgtataaagattgtgcaaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactctcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctattagatacatagatcctcgtcgcgatatcgcattttctaacgtgatggatatattaaagtcgaataaagtgaacaataattaattctttattgtcatcatgaacgggcgcgcctataaaaattgaaattttattttttttttttggaatataaatatccctatcagtgatagagatctccctatcagtgatagagagccaccatgtatcgaatgcaattactttcctgtatcgcactttcattagcccttgtgaccaactcagcgccaacatcaagttcgaccaagaagacgcagttgcagctagagcatttgcttttggatcttcaaatgatccttaatggtataaataattataagaaccccaaattgacgcgaatgctaacagctaaattcgcaatgccaaagaaggcaaccgagttaaagcacctacaatgcttggaagaagaactaaaaccccttgaggaggtattaaatggtgctcagtcgaagaattttcatcttcgacctcgagacctaatttcaaatattaacgtaattgttttggaattaaagggttcggaaactacttttatgtgtgagtacgcagacgagacagctacaatagtggagtttcttaaccgttggataaccttttgtcaatcaatcatttcgactttgacctaatgagcgatcgcgtgtagaaagtgttacatcgactcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatcggctcctggtacacatatacgttatttgagagatcatttctataatttaggaatgattatcaaatggatgctaattgacggacgccatcatgatcctattctaaatggattgcgtgatgtgactctagtgactcggttcgttgatgaggaatatctacgatccatcaaaaaacaactgcatccttctaagattattttaatttctgatgtaagatccaaacgaggaggaaatgaacctagtacggcggatttactaagtaattacgctctacaaaatgtcatgattagtattttaaaccccgtggcatctagtcttaaatggagatgcccgtttccagatcaatggatcaaggacttttatatcccacacggtaataaaatgttacaaccttttgctccttcatattcagctgaaatgaattc

SEQ ID NO: 19 - DNA, 507 bp, Artificial Sequence
  Synthetic Construct
atgtactcgatgcagttagcttcctgcgtgaccctaaccttagtcttgctagtgaattcggcgcccacctcatcctcaacgtcatcttccacagcggaggctcaacagcagcagcaacagcagcaacaacaacagcagcatttggaacaattgctaatggacttacaggaactactatcaagaatggagaattatcgaaacctaaagttacctcgaatgttgacagcaaaatttgcgttgccaaagcaggccacagagctaaaggacctacagtgtcttgaagatgagctaggaccacttcgtcacgttttagacggaacacagtccaagtcttttcagttggaagacgccgagaactttatatctaacatacgtgttactgtcgtaaaacttaaaggatcggacaatactttcgaatgccaattcgatgatgaaagtgcaaccgtcgtggacttcttgcgacgttggatcgccttctgtcaaagtataatttccacttcgccacag

SEQ ID NO: 20 - DNA, 2204 bp, Artificial Sequence
  Synthetic Construct
actttaatcctgtgtttatagagcccacgtttaaacattctttattaagtgtttataaacacagattaatagttttatttgaagtattcgttgtattcattctaatatatgtattttttagatctgaattaaatatgttcttcatgcctaaacgaaaaatacccgatcctattgatagattacgacgtgctaatctagcgtgtgaagacgataaattaatgatctatggattaccatggatgacaactcaaacatctgcgttatcaataaatagtaaaccgatagtgtataaagattgtgcaaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactttcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctgttagatacatagatcctcgtcgcaatatcgcattttctaacgtgatggatatattaaagtcgaataaagtgaacaataattaattctttattgtcatcggcgcgcctataaaaattgaaattttattttttttttttggaatataaatatccctatcagtgatagagatctccctatcagtgatagagagccaccatgtactcgatgcagttagcttcctgcgtgaccctaaccttagtcttgctagtgaattcggcgcccacctcatcctcaacgtcatcttccacagcggaggctcaacagcagcagcaacagcagcaacaacaacagcagcatttggaacaattgctaatggacttacaggaactactatcaagaatggagaattatcgaaacctaaagttacctcgaatgttgacagcaaaatttgcgttgccaaagcaggccacagagctaaaggacctacagtgtcttgaagatgagctaggaccacttcgtcacgttttagacggaacacagtccaagtcttttcagttggaagacgccgagaactttatatctaacatacgtgttactgtcgtaaaacttaaaggatcggacaatactttcgaatgccaattcgatgatgaaagtgcaaccgtcgtggacttcttgcgacgttggatcgccttctgtcaaagtataatttccacttcgccacagtattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatctgctcccggtacacatatacgttatttgagagatcatttctataatttaggagtgatcatcaaatggatgctaattgacggccgccatcatgatcctattttaaatggattgcgtgatgtgactctagtgactcggttcgttgatgaggaatatctacgatccatcaaaaaacaactgcatccttctaagattattttaatttctgatgtgagatccaaacgaggaggaaatgaacctagtacggcggatttactaagtaattacgctctacaaaatgtcatgattagtattttaaaccccgtggcgtctagtcttaaatggagatgcccgtttccagatcaatggatcaaggacttttatatcccacacggtaataaaatgttacaaccttttgctccttcatattcagctgaaatgagattattaagtatttataccggtgagaacatgagactgactcgagttaccaaatcagacgctgtaaattatgaaaaaaagatgtactaccttaataagatcgtccgtaacaa

SEQ ID NO: 21 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 22 - protein, 20 aa, Homo sapians
MYRMQLLSCIALSLALVTNS

SEQ ID NO: 23 - protein, 149 aa, Homo sapiens
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 24 - protein, 169 aa, Mus musculus
MYSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 25 - protein, 376 aa, human herpesvirus 1
MASYPGHQHASAFDQAARSRGHSNRRTALRPRRQQEATEVRPEQKMPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMIQTHVTTPGSIPTICDLARTFAREMGEAN

SEQ ID NO: 26 - protein, 376 aa, Artificial Sequence
  Synthetic Construct
MASYPGHQHASAFDQAARSRGHSNRRTALRPRRQQEATEVRPEQKMPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHVPPPALTILADRHPIAYFLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMIQTHVTTPGSIPTICDLARTFAREMGEAN

SEQ ID NO: 27 - protein, 376 aa, Artificial Sequence
  Synthetic Construct
MASYPGHQHASAFDQAARSRGHSNRRTALRPRRQQEATEVRPEQKMPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTIFLDRHPIAFMLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMIQTHVTTPGSIPTICDLARTFAREMGEAN

SEQ ID NO: 28 - protein, 376 aa, Artificial Sequence
  Synthetic Construct
MASYPGHQHASAFDQAARSRGHSNRRTALRPRRQQEATEVRPEQKMPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTLIFDRHPIAHLLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMIQTHVTTPGSIPTICDLARTFAREMGEAN

(19) United States
(12) Patent Application Publication
(10) Pub. No.: US 2023/0201283 A1
(43) Pub. Date: Jun. 29, 2023

(54) RECOMBINANT VACCINIA VIRUS

(71) Applicant: Pfizer Inc., New York, NY (US)

(72) Inventors: Joseph John Binder, San Diego, CA (US); Michael Dale Eisenbraun, San Diego, CA (US); Douglas Hanahan, Saint-Sulpice (CH); David H. Kirn, Mill Valley, CA (US); Clare Lees, Del Mar, CA (US); Prajit Limsirichai, Washington, DC (US); Liliana Maruri Avidal, Oakland, CA (US)

(21) Appl. No.: 17/790,160

(22) PCT Filed: Jan. 5, 2021
(86) PCT No.: PCT/IB2021/050040

§ 371 Date: Jun. 30, 2022

Related U.S. Application Data
(60) Provisional application No. 62/959,083, filed on Jan. 9, 2020.

(51) Int. Cl. A61K 35/768; C12N 15/86; A61K 39/395; C07K 14/55; C12N 9/12; A61P 35/00

(52) U.S. Cl. CPC A61K 35/768; A61K 39/3955; A61P 35/00; C07K 14/55; C12N 9/1211; C12N 15/86; C12Y 207/01021; C12N 2710/24132; C12N 2710/24143

(57) ABSTRACT

The present disclosure provides a replication-competent, recombinant oncolytic vaccinia virus (RVV), compositions comprising the RVV, and use of the RVV or composition for inducing oncolysis in an individual having a tumor.

### CROSS-REFERENCE TO RELATED APPLICATIONS

This application claims the benefit of U.S. Provisional Application No. 62/959,083 filed Jan. 9, 2020, the disclosure of which is incorporated herein by reference in its entirety.

### INCORPORATION BY REFERENCE OF SEQUENCE LISTING PROVIDED AS A TEXT FILE

A Sequence Listing is provided herewith as a text file, “PC72576A_SEQ_LISTING_ST25.txt” created on Dec. 3, 2020 and having a size of 69 KB. The contents of the text file are incorporated by reference herein in their entirety.

### BACKGROUND

Oncolytic viruses (OVs) are viruses that selectively or preferentially infect and kill cancer cells. Live replicating OVs have been tested in clinical trials in a variety of human cancers. OVs can induce anti-tumor immune responses, as well as direct lysis of tumor cells (i.e., oncolysis). OVs can occur naturally or can be constructed by modifying other viruses. Common OVs include those that are constructed based-on attenuated strains of Herpes Simplex Virus (HSV), Adenovirus (Ad), Measles Virus (MV), Coxsackie virus (CV), Vesicular Stomatitis Virus (VSV), and Vaccinia Virus (VV).

VV is a member of the orthopoxvirus genus of the poxvirus family. It has a linear, double-stranded DNA genome approximately 190 kb in length, which encodes about 200 genes. VV replicates in the cytoplasm of a host cell. The large VV genome codes for various enzymes and proteins used for viral DNA replication. During replication, VV produces several infectious forms which differ in their outer membranes: the intracellular mature virion (IMV), the intracellular enveloped virion (IEV), the cell-associated enveloped virion (CEV) and the extracellular enveloped virion (EEV). IMV is the most abundant infectious form and is thought to be responsible for spread between hosts; the CEV is believed to play a role in cell-to-cell spread; and the EEV is thought to be important for long range dissemination within the host organism. EEV-specific proteins are encoded by the genes A33R, A34R, A36R, A56R, B5R, and F13L, A34, a type II transmembrane glycoprotein encoded by the A34R gene, is involved in the induction of actin tails, the release of enveloped virus from the surfaces of infected cells, and the disruption of the virus envelope after ligand binding prior to virus entry.

VV is one of the commonly used backbones for oncolytic virus engineering due to its long history of use as the routine vaccine for smallpox. Clinical data suggest that the efficacy of oncolytic vaccinia virus (OVV) treatment tends to be dose-dependent. Therefore, in clinical settings, a high treatment dosage (substantially higher than vaccination dose) is more likely to be applied to maximize the OVV’s anti-tumor effects. However, high-level, persistent viral replication may cause major safety concerns when dealing with replication-competent oncolytic viruses, including OVV, as addressed by the US FDA in the guidance of Preclinical Assessment of Investigational Cellular and Gene Therapy Products (November 2013). Therefore, there is a need for a replication-controllable OVV and a method of controlling OVV replication in a patient administered with the OVV.

### SUMMARY

The present disclosure provides a replication-competent, recombinant oncolytic vaccinia virus (hereinafter referred to as “recombinant vaccinia virus,” “recombinant VV,” or “RVV,”) which comprises: (i) a nucleotide sequence encoding an immunostimulatory cytokine polypeptide, such as interleukin-2 (IL-2) polypeptide or a variant thereof (IL-2v); and (ii) a nucleotide sequence encoding a heterologous thymidine kinase (TK) polypeptide. In some embodiments, the present disclosure provides a replication-controllable RVV comprising a herpes simplex virus thymidine kinase (HSV-tk) polypeptide, which allows for viral replication control via an anti-viral agent, such as a 2′-deoxyguanosine analog (e.g., such as ganciclovir or GCV). The present disclosure further provides compositions comprising the RVVs, methods of inducing oncolysis in an individual having a tumor comprising administering to the individual an effective amount of the RVV or a composition of the present disclosure and use of an RVV or composition of the present disclosure in the manufacture of a medicament for treatment of cancers or inducing oncolysis. The disclosure also provides methods of controlling the replications of the RVVs, or reducing the side effects caused by the RVV, in a subject administered the virus, comprising administering to the subject an effective amount a 2′-deoxyguanosine analog, such as ganciclovir.

### BRIEF DESCRIPTION OF THE DRAWINGS

FIG. 1 provides schematic representation of full genomes for representative recombinant vaccinia viruses VV91, VV93, and VV96. Abbreviations: LITR = left inverted terminal repeat; RITR = right inverted terminal repeat; A - O = viral gene regions historically defined by HindIII digest fragments; P_{SEL} = synthetic early late promoter; mIL2v = mouse interleukin-2 variant; * = mutation encoding substitution of lysine to glutamate at position 151 of A34 protein; P_{F17} = promoter from the F17R gene; HSV TK.007 = herpes simplex virus thymidine kinase gene with mutation encoding alanine to histidine substitution at position 168.

FIG. 2 provides schematic representation of full genomes for representative recombinant vaccinia viruses VV94 and IGV121. Abbreviations: LITR = left inverted terminal repeat; RITR = right inverted terminal repeat; A - O = viral gene regions historically defined by HindIII digest fragments; P_{SEL} = synthetic early late promoter; mIL2v = mouse interleukin-2 variant; * = mutation encoding substitution of lysine to glutamate at position 151 of A34 protein; P_{F17} = promoter from the F17R gene; HSV TK.007 = herpes simplex virus thymidine kinase gene with mutation encoding alanine to histidine substitution at position 168.

FIG. 3 provides schematic representation of full genomes for recombinant vaccinia viruses VV101, VV102, and VV103. Abbreviations: LITR = left inverted terminal repeat; RITR = right inverted terminal repeat; A - O = viral gene regions historically defined by HindIII digest fragments; P_{SEL} = synthetic early late promoter; hIL2v = human interleukin-2 variant; * = mutation encoding substitution of lysine to glutamate at position 151 of A34 protein; P_{F17} = promoter from the F17R gene; HSV TK.007 = herpes simplex virus thymidine kinase gene with mutation encoding alanine to histidine substitution at position 168.

FIG. 4 provides results of mouse IL-2 variant (mIL-2v) expression analysis following infection of cells with recombinant oncolytic vaccinia viruses.

FIG. 5 provides results of human IL-2 variant (hIL-2v) expression analysis following infection of cells with recombinant oncolytic vaccinia viruses.

FIG. 6 provides results of HSV TK.007 mRNA expression analysis following infection of cells with recombinant oncolytic vaccinia viruses.

FIGS. 7A-7G provide results of assessment of virotherapy-induced tumor growth inhibition on C57BL/6 female mice implanted SC with MC38 tumor cells. Tumor growth trajectories are shown for individual mice in groups treated with vehicle only (A) or Copenhagen vaccinia virus containing the A34R K151E mutation armed with either a Luciferase-2A-GFP reporter (Cop.Luc-GFP.A34R-K151E; VV16) (B), mIL-2v only (Cop.mGM-CSF.A34R-K151E; VV27) (C), mIL-2v and HSV TK.007 in a forward orientation in the B16R gene locus (Cop.mIL-2v.A34R-K151E.HSV TK.007 (B16R_For); VV91) (D), mIL-2v and HSV TK.007 in a reverse orientation in the J2R gene locus (Cop.mIL-2v.A34R-K151E.HSV TK.007 (J2R_Rev); VV93) (E), or mIL-2v and HSV TK.007 in a reverse orientation in the B16R gene locus (Cop.mIL-2v.A34R-K151E.HSV TK.007 (B16R_Rev); VV96) (F). The dashed vertical line on each graph represents time point when mice received intratumoral injections of vehicle or virus. The dashed horizontal line on each graph represents the tumor volume threshold used as a criterion to remove animals from the study. Average tumor volumes (mm3) ± 95% confidence intervals for each treatment group are shown through day 28 post-tumor implant (G), which was the last tumor measurement time point when all animals in each group were still alive.

FIG. 8 provides results of statistical comparison of virotherapy-induced tumor growth inhibition using ANCOVA. Tumor volumes for individual mice in each group after vehicle/virus treatment (day 14 to day 27 post-tumor implantation) were analyzed by ANCOVA to determine statistically significant inhibitory effects on tumor growth across various treatment groups. Columns show the statistical results (p values) of comparisons between specific treatment group pairs. Values in bold font represent comparative ANCOVA results where p ≤ 0.05.

FIG. 9 provides results of a representative study on survival of MC38 tumor-implanted C57BL/6 female mice following treatment with vehicle or virus on day 12 after implantation. Mice were designated daily as deceased upon reaching tumor volume ≥ 1400 mm3. The point of intersection between each group’s curve and the horizontal dashed line indicates the median (50%) survival threshold for group.

FIG. 10 provides results of IL-2 levels detected in sera collected from MC38 tumor-bearing C57BL/6 female mice 24 hours and 48 hours after intratumoral injection with vehicle or recombinant Cop vaccinia viruses. Each symbol represents the calculated IL-2 serum levels for an individual mouse, while bars represent group geometric mean (N=9 /group). Error bars represent 95% confidence intervals.

FIGS. 11A-11F provide results of assessment of virotherapy-induced tumor growth inhibition on C57BL/6 female mice implanted SC with LLC tumor cells. Tumor growth trajectories are shown for individual mice in groups treated with vehicle only (A) or Copenhagen vaccinia virus containing the A34R K151E mutation and armed with either a Luciferase-2A-GFP reporter (Cop.Luc-GFP.A34R-K151E; VV16) (B), mIL-2v only (Cop.IL-2v.A34R-K151E; VV27) (C), mIL-2v and HSV TK.007 in a forward orientation in the B16R gene locus (Cop.mIL-2v.A34R-K151E.HSV TK.007 (B16R_For); VV91) (D), mIL-2v and HSV TK.007 in a reverse orientation in the J2R gene locus (Cop.mIL-2v.A34R-K151E.HSV TK.007 (J2R_Rev); VV93) (E), mIL-2v and HSV TK.007 in a reverse orientation in the B16R gene locus (Cop.mIL-2v.A34R-K151E.HSV TK.007 (B16R_Rev); VV96) (F). The dashed vertical line on each graph represents time point when mice received intratumoral injections of vehicle or virus. The dashed horizontal line on each graph represents the tumor volume threshold used as a criterion to remove animals from the study.

FIG. 12 provides results of IL-2 levels detected in sera collected from LLC tumor-bearing C57BL/6 female mice 24, 48, and 72 hours after intratumoral injection with vehicle or recombinant Cop vaccinia viruses. Each symbol represents the calculated IL-2 serum levels for an individual mouse, while bars represent group geometric mean (N=5 /group). Error bars represent 95% confidence intervals.

FIGS. 13A-13F provide results of assessment of virotherapy-induced tumor growth inhibition using single (day 11) IV virus delivery on C57BL/6 female mice implanted SC with MC38 tumor cells. Tumor growth trajectories are shown for each treatment as group averages ± 95% confidence intervals up through day 32 post-tumor implantation until time of sacrifice (A) or for individual mice in each group until time of sacrifice or study termination (B-F). Test viruses included WR vaccinia viruses containing the A34R K151E mutation and armed with either a Luciferase-2A-GFP reporter (WR.Luc-GFP.A34R-K151E; VV17) (C), mIL-2v only (WR.mIL-2v.A34R-K151E; VV79) (D), mIL-2v with HSV TK.007 in a reverse orientation in the J2R gene locus (WR.mIL-2v.A34R-K151E.HSV TK.007 (J2R_Rev); VV94) (E), and mIL-2v and HSV TK.007 in a forward orientation in the B15R/B17R gene locus (WR.mIL-2v.A34R-K151E.HSV TK.007 (B16R_For); IGV-121) (F). Dashed vertical lines on each graph represent time points when mice received IV injections of virus. The dashed horizontal line on each graph represents the tumor volume threshold used as a criterion to remove animals from the study.

FIG. 14 provides results of statistical comparison of virotherapy-induced tumor growth inhibition using ANCOVA for subcutaneous MC38 tumor model study. Tumor volumes for individual mice in each group on multiple days after treatment were analyzed by ANCOVA to determine statistically significant inhibitory effects on tumor growth across various treatment groups. Columns show the statistical results (p values) of comparisons between specific treatment group pairs. Values in bold font represent comparative ANCOVA results where p values ≤ 0.05 were observed.

FIG. 15 provides results of survival of MC38 tumor-bearing C57BL/6 female mice following IV treatment with recombinant oncolytic vaccinia viruses on day 11 after SC tumor implantation. Mice were designated daily as deceased upon reaching tumor volume ≥ 1400 mm3. The point of intersection between each group’s curve and the horizontal dashed line indicates the median (50%) survival threshold for the group. P values represent the statistical results of Log-rank test (Mantel-Cox) comparisons between select virus groups.

FIG. 16 provides results of IL-2 levels detected in sera collected from MC38 tumor-bearing C57BL/6 female mice 72 hours (day 14) after IV injection with 5e7 pfu recombinant WR vaccinia viruses. Each symbol represents IL-2 serum levels detected in an individual mouse, while bars represent the group geometric means (N=10/group). Error bars represent 95% confidence intervals.

FIGS. 17A-17D provide results of assessment of virotherapy-induced tumor growth inhibition using single (day 14) IV virus delivery on C57BL/6 female mice implanted SC with LLC tumor cells. Tumor growth trajectories are shown for each treatment as group averages ± 95% confidence intervals up through day 27 post-tumor implantation until time of sacrifice (A) or for individual mice in each group until time of sacrifice or study termination (B-D). Test viruses included WR vaccinia viruses armed with either a Luciferase-2A-GFP reporter (WR.Luc-GFP; VV3) (C), or mIL-2v and HSV TK.007 in a forward orientation in the B15R/B17R gene locus with the A34R K151E mutation (WR.mIL-2v.A34R-K151E.HSV TK.007 (B16R_For); IGV-121)) (D). Dashed vertical lines on each graph represent time points when mice received IV injections of virus. The dashed horizontal line on each graph represents the tumor volume threshold used as a criterion to remove animals from the study.

FIG. 18 provides results of statistical comparison of virotherapy-induced tumor growth inhibition using ANCOVA for subcutaneous LLC tumor model study. Tumor volumes for individual mice in each group on multiple days after treatment were analyzed by ANCOVA to determine statistically significant inhibitory effects on tumor growth across various treatment groups. Columns show the statistical results (p values) of comparisons between specific treatment group pairs. Values in bold font represent comparative ANCOVA results where p values ≤ 0.05 were observed.

FIG. 19 provides results of survival of LLC tumor-bearing C57BL/6 female mice following IV treatment with recombinant oncolytic vaccinia viruses on day 11 after SC tumor implantation. Mice were designated daily as deceased upon reaching tumor volume ≥ 2000 mm3. The point of intersection between each group’s curve and the horizontal dashed line indicates the median (50%) survival threshold for the group. P values represent the statistical results of Log-rank test (Mantel-Cox) comparisons between select virus groups.

FIGS. 20A-20I provide results of assessment of virotherapy-induced tumor growth inhibition on C57BL/6 female mice implanted SC with MC38 tumor cells. Tumor growth trajectories are shown for individual mice in groups treated with vehicle only (A), Copenhagen vaccinia virus armed with either mIL-2v and HSV TK.007 in a forward orientation in the B16R gene locus (Cop.mIL-2v.A34R-K151E.HSV TK.007 (B16R_For); VV91)) at 5e7 pfu (B), hIL-2v and HSV TK.007 in a forward orientation in the B16R gene locus (Cop.hIL-2v.A34R-K151E.HSV TK.007 (B16R_For); VV102)) at 5e7 pfu (C), mGM-CSF and a LacZ reporter transgene (Cop.mGM-CSF/LacZ; (VV10) at 5e7 pfu (D), a Luciferase-2A-GFP reporter (Cop.Luc-GFP; VV7) at 2e8 pfu (E), mIL-2v and HSV TK.007 in a forward orientation in the B16R gene locus (Cop.mIL-2v.A34R-K151E.HSV TK.007 (B16R_For); VV91)) at 2e8 pfu (F), hIL-2v and HSV TK.007 in a forward orientation in the B16R gene locus (Cop.hIL-2v.A34R-K151E.HSV TK.007 (B16R_For); W102)) at 2e8 pfu (G), and mGM-CSF and a LacZ reporter transgene (Cop.mGM-CSF/LacZ; (VV10) at 2e8 pfu (H). The dashed vertical line on each graph represents time point when mice received intratumoral injections of vehicle or virus. The dashed horizontal line on each graph represents the tumor volume threshold used as a criterion to remove animals from the study. Average tumor volumes (mm³) for each treatment group are shown through day 28 post-tumor implant (I).

FIG. 21 provides results of statistical comparison of virotherapy-induced tumor growth inhibition using ANCOVA. Tumor volumes for individual mice in each group after vehicle/virus treatment (day 14 to day 28 post-tumor implantation) were analyzed by ANCOVA to determine statistically significant inhibitory effects on tumor growth across various treatment groups. Columns show the statistical results (p values) of comparisons between specific treatment group pairs. Values in bold font represent comparative ANCOVA results where p ≤ 0.05.

FIGS. 22A -22B provide results of survival of MC38 tumor-implanted C57BL/6 female mice following treatment with vehicle or recombinant vaccinia virus on day 11 after implantation. Mice were designated daily as deceased upon reaching tumor volume ≥ 1400 mm3. The point of intersection between each group’s curve and the horizontal dashed line indicates the median (50%) survival threshold for group. (A) shows groups dosed with 5e7 pfu virus. (B) shows groups dosed with virus at 2e8 pfu.

FIG. 23 provides results of mouse IL-2 levels detected in sera collected from MC38 tumor-bearing C57BL/6 female mice 24 hours after intratumoral injection with vehicle or recombinant Cop vaccinia viruses. Each symbol represents the calculated IL-2 serum levels for an individual mouse, while bars represent group geometric mean (N=10 /group). Error bars represent 95% confidence intervals.

FIG. 24 provides results of human IL-2 levels detected in sera collected from MC38 tumor-bearing C57BL/6 female mice 24 hours after intratumoral injection with vehicle or recombinant Cop vaccinia viruses. Each symbol represents the calculated IL-2 serum levels for an individual mouse, while bars represent group geometric mean (N=9 /group). Error bars represent 95% confidence intervals.

FIG. 25 provides results of assessment of virotherapy-induced tumor growth inhibition on athymic nude female mice implanted SC with HCT-116 tumor cells after intratumoral injection with vehicle or recombinant Cop vaccinia viruses. Average tumor volumes (mm3) for each treatment group are shown through day 40 post-tumor implant. The dashed vertical line on each graph represents time point when mice received intratumoral injections of vehicle or virus. The dashed horizontal line on each graph represents the tumor volume threshold used as a criterion to remove animals from the study.

### DETAILED DESCRIPTION

### A. Definitions

The term “heterologous” refers to a molecule (e.g., a nucleic acid, polypeptide, protein, or gene) that is not found in a naturally-occurring organism. For example, in the context of a recombinant oncolytic vaccinia virus of the present disclosure, a nucleic acid comprising a nucleotide sequence encoding a “heterologous” thymidine kinase polypeptide refers to a thymidine kinase polypeptide, such as a thymidine kinase polypeptide from herpes simplex virus (HSV), which is not found in naturally-occurring vaccinia virus.

The term “oncolytic virus” refers to a virus that preferentially infects and kills cancer cells (oncolysis), compared to normal (non-cancerous) cells.

The term “replication-competent” refers to a virus that is capable of infecting and replicating within a particular host cell.

The term “recombinant” virus refers to a virus that is constructed based on a wild-type or existing virus (i.e., parent virus), using recombinant nucleic acid techniques, by introducing changes or modifications to the viral genome and/or to introduce changes or modifications to the viral proteins. For example, a recombinant virus may contain modified endogenous nucleic acid sequences, exogenous nucleic acid sequences, or both. A recombinant virus may also include modified protein components. A “recombinant vaccinia virus” refers to a recombinant virus that is modified or constructed based on a wild-type or existing vaccinia virus.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “individual,” “subject,” “host,” and “patient,” used interchangeably herein, refer to a mammal, including, but not limited to, murines (e.g., rats, mice), lagomorphs (e.g., rabbits), non-human primates, humans, canines, felines, ungulates (e.g., equines, bovines, ovines, porcines, caprines).

The term “substitution” refers to the replacement of one amino acid in a polypeptide with a different amino acid. In the context of the present disclosure, a substitution in a polypeptide is indicated as: original amino acid-position-substituted amino acid. Accordingly, the notation “K151E” means, that the variant comprises a substitution of Lysine (K) with Glutamic acid (E) in the variant amino acid position corresponding to the amino acid in position 151 in the parent polypeptide.

A “therapeutically effective amount” or “efficacious amount” refers to the amount of an agent (e.g., a replication-competent, recombinant oncolytic vaccinia virus of the present disclosure), or combined amounts of two agents (e.g., a replication-competent, recombinant oncolytic vaccinia virus of the present disclosure and a second therapeutic agent), that, when administered to a subject for treating a disease, is sufficient to cause an intended effect, such treatment for the disease. The “therapeutically effective amount” will vary depending on the agent(s), the disease and its severity and the age, weight, etc., of the subject to be treated.

The terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The term “variant” polypeptide refers to a polypeptide that contains one or more amino acid mutations relative to the amino acid sequence of a reference polypeptide and retains certain properties of the reference polypeptide. The variant may be arrived at by modification of the amino acid sequence of the reference polypeptide by such modifications as insertion, substitution, or deletion of one or more amino acids. Accordingly, the term “variant” polypeptide encompasses fragments of a reference polypeptide that comprises a sufficient number of contiguous amino acid residues to confer a desired biological property.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a vaccinia virus” includes a plurality of such vaccinia viruses and reference to “the variant IL-2 polypeptide” includes reference to one or more variant IL-2 polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all subcombinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### B. Recombinant Oncolytic Vaccinia Virus

In a first aspect, the present disclosure provides a replication-competent, recombinant oncolytic vaccinia virus having modifications in the viral genome or viral proteins relative to the corresponding wild-type or existing virus (i.e., parent virus), wherein the modifications comprise: (i) an inserted nucleotide sequence encoding an immunostimulatory cytokine polypeptide; and (ii) an inserted nucleotide sequence encoding a heterologous thymidine kinase polypeptide. For convenience, the replication-competent, recombinant oncolytic vaccinia virus provided by the present disclosure may be referred to as “recombinant vaccinia virus” or “RVV.” In some embodiments, the RVV further comprises one or more modifications or mutations to the native genome, protein, or other components of the virus, which increases or enhances one or more desirable anti-tumor properties of the virus, such as increased tumor selectivity, increased oncolysis properties, enhanced production of extracellular enveloped virus (EEV), or reduced toxicity. Examples of insertions and other modifications found in an RVV provided by the present disclosure are described in detail herein below.

### B-1. Immunostimulatory Cytokine Polypeptides

As noted above, the RVV provided by the present disclosure comprises an inserted nucleotide sequence encoding an immunostimulatory cytokine polypeptide. The term “immunostimulatory cytokine” refers to a cytokine that is capable of stimulating expansion of cytotoxic T cells in the presence of IL-2 receptor, enhancing innate or adoptive immunity against a tumor, or otherwise enhancing the anti-cancer activity of an oncolytic virus. Examples of immunostimulatory cytokines include interleukin (IL)-2 (IL-2; also known as T-cell growth factor), IL-6, IL-12, IL-15, IL-18 (also known as IFN-γ-inducing factor), IL24, and GM-CFF.

In some embodiments, the RVV comprises a nucleotide sequence encoding a wild-type IL-2 polypeptide or a variant thereof. A variant of a wild-type IL-2 polypeptide may also be referred to herein an “IL-2v polypeptide.” In an embodiment, the RVV comprises a nucleotide sequence encoding an IL-2v polypeptide that, when expressed in a subject being administered the recombinant VV, has reduced toxicity, reduced binding to receptor CD25 (high-affinity IL-2 receptor α (alpha) subunit), reduced stimulation of immunosuppressive T-regulatory cells (T-reg cells), or otherwise reduced immunosuppressive activities. In a particular embodiment, the IL-2v polypeptide comprises an amino acid substitution that provides for reduced binding to CD25 compared to wild-type IL-2. The IL-2v polypeptide-encoding nucleotide sequence is present in the genome of the RVV and may be referred to as a “transgene.” The IL-2v polypeptide-encoding nucleotide sequence is not normally present in wild-type vaccinia virus and is thus heterologous to wild-type vaccinia virus. Thus, the IL-2v polypeptide-encoding nucleotide sequence can be referred to as a “heterologous nucleotide sequence” or “inserted nucleotide sequence” encoding an IL-2v polypeptide.” A virus comprising a transgene is said to be “armed” with the transgene. Thus, an RVV of the present disclosure that comprises a nucleotide sequence encoding an IL-2v polypeptide may be said to be “armed” with the IL-2v-encoding nucleotide sequence.

In some embodiment, the IL-2 polypeptide is a human IL-2 polypeptide or a variant thereof. In some other embodiments, the IL-2 polypeptide is a mouse IL-2 polypeptide or a variant thereof. The amino acid sequence of the mature form of a wild-type human IL-2 (hIL-2) polypeptide is set forth in SEQ ID NO: 1. The amino acid sequence of the precursor form of the wild-type hIL-2 polypeptide is set forth in SEQ ID NO:21. The precursor form of the wild-type hIL-2 polypeptide includes a signal peptide (e.g., MYRMQLLSCIALSLALVTNS (SEQ ID NO:22)). The amino acid sequence of the mature form of a wild-type mouse IL-2 (mIL-2) polypeptide is set forth in SEQ ID NO:23. The amino acid sequence of the precursor form of the mouse wild-type IL-2 polypeptide is set forth in SEQ ID NO:24.

In some cases, an IL-2v polypeptide encoded by an RVV of the present disclosure provides reduced undesirable biological activity when compared to wild-type IL-2. In some cases, said reduced undesirable biological activity is determined by measuring potency at inducing increased pSTAT5 levels in CD25+ CD4+ Treg cells when compared to wild-type IL-2. In some cases, an IL-2v polypeptide provides reduced concentration potency when compared to wild-type IL-2 at inducing increased pSTAT5 levels in CD25+ CD4+ Treg cells. In some cases, an IL-2v polypeptide provides reduced concentration potency of at least 1, at least 2 or at least 3 logs when compared to wild-type IL-2 at inducing increased pSTAT5 levels in CD25+ CD4+ Treg cells. In some cases, an IL-2v polypeptide provides reduced concentration potency of about 1, about 2 or about 3 logs when compared to wild-type IL-2 at inducing increased pSTAT5 levels in CD25+ CD4+ Treg cells. In some cases, said reduced undesirable biological activity is determined by measuring the proinflammatory cytokine levels after treatment with an IL-2v polypeptide encoded by the RVV when compared to wild-type IL-2, as disclosed at Example 9. In some cases, an IL-2v polypeptide provides reduced proinflammatory cytokine levels when compared to wild-type IL-2 (e.g. using the test disclosed at Example 9). In some cases, an IL-2v polypeptide provides reduced proinflammatory cytokine levels by at least 10%, at least 15%, at least 20%, at least 25%, at least 30%, at least 40%, at least 45%, at least 50%, at least 55%, at least 60%, at least 65%, at least 70%, at least 75%, at least 80%, at least 85%, at least 90%, at least 95%, or at least 100%, when compared to wild type IL-2.

In some cases, an IL-2v polypeptide comprises a substitution of one or more of F42, Y45, and L72, based on the amino acid numbering of the IL-2 amino acid sequence depicted in SEQ ID NO:1. In some cases, an IL-2v polypeptide comprises a substitution of one or more of F42 and Y45, based on the amino acid numbering of the IL-2 amino acid sequence depicted in SEQ ID NO:1. In some cases, an IL-2v polypeptide comprises a substitution of one or more of F42 and L72, based on the amino acid numbering of the IL-2 amino acid sequence depicted in SEQ ID NO:1. In some cases, an IL-2v polypeptide comprises a substitution of one or more of Y45 and L72, based on the amino acid numbering of the IL-2 amino acid sequence depicted in SEQ ID NO:1. In some cases, an IL-2v polypeptide comprises an F42L, F42A, F42G, F42S, F42T, F42Q, F42E, F42D, F42R, or F42K substitution, based on the amino acid numbering of the IL-2 amino acid sequence depicted in SEQ ID NO: 1. In some cases, an IL-2v polypeptide comprises a Y45A, Y45G, Y45S, Y45T, Y45Q, Y45E, Y45N, Y45D, Y45R, or Y45K substitution, based on the amino acid numbering of the IL-2 amino acid sequence depicted in SEQ ID NO: 1. In some cases, an IL-2v polypeptide comprises an L72G, L72A, L72S, L72T, L72Q, L72E, L72N, L72R, or L72K substitution, based on the amino acid numbering of the IL-2 amino acid sequence depicted in SEQ ID NO: 1.

In some cases, an RVV of the present disclosure comprises a nucleotide sequence encoding an IL-2v polypeptide that includes a signal peptide (e.g., MYRMQLLSCIALSLALVTNS (SEQ ID NO:22). Thus, e.g., in some cases, a replication-competent, recombinant oncolytic vaccinia virus of the present disclosure comprises a nucleotide sequence encoding an IL-2v polypeptide having at least 95% (e.g., at least 95%, at least 98%, at least 99%, or 100%) amino acid sequence identity to the IL-2 amino acid sequence depicted in SEQ ID NO:21, and comprising a substitution of one or more of F62, Y65, and L92 of the IL-2 based on the amino acid numbering of the amino acid sequence depicted in SEQ ID NO:21. As will be appreciated, F62, Y65, and L92 of the IL-2 amino acid sequence depicted in SEQ ID NO:21 correspond to F42, Y45, and L72 of the amino acid sequence depicted in SEQ ID NO: 1.

In some cases, an IL-2v polypeptide encoded by an RVV of the present disclosure comprises one or more of: a) an F42L, F42A, F42G, F42S, F42T, F42Q, F42E, F42D, F42R, or F42K substitution; b) a Y45A, Y45G, Y45S, Y45T, Y45Q, Y45E, Y45N, Y45D, Y45R, or Y45K substitution; and c) an L72G, L72A, L72S, L72T, L72Q, L72E, L72N, L72R, or L72K substitution, based on the amino acid numbering of the IL-2 amino acid sequence depicted in SEQ ID NO:1. In some cases, an IL-2v polypeptide comprises: a) an F42L, F42A, F42G, F42S, F42T, F42Q, F42E, F42D, F42R, or F42K substitution; and b) a Y45A, Y45G, Y45S, Y45T, Y45Q, Y45E, Y45N, Y45D, Y45R, or Y45K substitution, based on the amino acid numbering of the IL-2 amino acid sequence depicted in SEQ ID NO:1. In some cases, an IL-2v polypeptide comprises: a) an F42L, F42A, F42G, F42S, F42T, F42Q, F42E, F42D, F42R, or F42K substitution; and b) an L72G, L72A, L72S, L72T, L72Q, L72E, L72N, L72R, or L72K substitution, based on the amino acid numbering of the IL-2 amino acid sequence depicted in SEQ ID NO:1. In some cases, an IL-2v polypeptide comprises: a) a Y45A, Y45G, Y45S, Y45T, Y45Q, Y45E, Y45N, Y45D, Y45R, or Y45K substitution; and b) an L72G, L72A, L72S, L72T, L72Q, L72E, L72N, L72R, or L72K substitution, based on the amino acid numbering of the IL-2 amino acid sequence depicted in SEQ ID NO:1. In some cases, an IL-2v polypeptide comprises: a) an F42L, F42A, F42G, F42S, F42T, F42Q, F42E, F42D, F42R, or F42K substitution; b) a Y45A, Y45G, Y45S, Y45T, Y45Q, Y45E, Y45N, Y45D, Y45R, or Y45K substitution; and c) an L72G, L72A, L72S, L72T, L72Q, L72E, L72N, L72R, or L72K substitution, based on the amino acid numbering of the IL-2 amino acid sequence depicted in SEQ ID NO: 1.

In some cases, the amino acid sequence of an IL-2v polypeptide comprises: (1) one or more substitutions of F42A, Y45A, and L72G; (2) one or both substitutions of F42A and Y45A; (3) substitutions of F42A and L72G; (4) substitutions of Y45A, and L72G; or (5) substitutions of F42A, Y45A, and L72G, wherein the amino acid numbering of the IL-2v polypeptide is based on the amino acid sequence of SEQ ID NO: 1.

In some cases, an IL-2v polypeptide encoded by an RVV of the present disclosure comprises an amino acid sequence having at least 95% (e.g., at least 95%, at least 98%, at least 99%, or 100%) amino acid sequence identity to the amino acid sequence depicted in SEQ ID NO:1, and comprises an amino acid substitution selected from the group consisting of:
(1) an F42A substitution;
(2) a Y45A substitution;
(3) an L72G substitution;
(4) an F42A substitution and an L72G substitution;
(5) an F42A substitution and a Y45A substitution;
(6) a Y45A substitution and an L72G substitution; and
(7) an F42A substitution, a Y45A substitution, and an L72G substitution,
wherein the amino acid numbering of the IL-2v polypeptide is based on the amino acid sequence of SEQ ID NO: 1.

In some cases, an IL-2v polypeptide encoded by a replication-competent RVV of the present disclosure does not include a substitution of T3 and/or C125. In other words, in some cases, an IL-2v polypeptide comprises a Thr at amino acid position 3, and a Cys at amino acid position 125, based on the amino acid numbering of the IL-2 amino acid sequence depicted in SEQ ID NO: 1.

Suitable amino acid sequences of IL-2v polypeptides include, e.g., a mouse IL-2v polypeptide comprising an amino acid sequence having at least 95% (e.g., at least 95%, at least 98%, at least 99%, or 100%) amino acid sequence identity to the following amino acid sequence:

MYSMQLASCVTLTLVLLVNSAPTSSSTSSSTAEAQQQQQQQQQQQQHLEQ
LLMDLQELLSRMENYRNLKLPRMLTAKFALPKQATELKDLQCLEDELGPL
RHVLDGTQSKSFQLEDAENFISNIRVTVVKLKGSDNTFECQFDDESATVV
DFLRRWIAFCQSIISTSPQ (SEQ ID NO:3)

, and comprising F76A, Y79A, and L106G substitutions (i.e., comprising Ala-76, Ala-79, and Gly-106).

Suitable nucleotide sequences encoding an IL-2v polypeptide include, e.g., a nucleotide sequence encoding a mouse IL-2v polypeptide and having at least 95% (e.g., at least 95%, at least 98%, at least 99%, or 100%) nucleotide sequence identity to the following nucleotide sequence:

ATGTACAGCATGCAGCTGGCCAGCTGCGTGACACTGACCCTCGTGCTGCT
GGTGAACAGCGCTCCTACCTCCTCCAGCACCAGCAGCAGCACCGCTGAGG
CCCAGCAGCAGCAGCAGCAACAGCAACAGCAGCAACAACATTTAGAACAG
CTGCTGATGGATTTACAAGAACTGCTGTCTCGTATGGAGAACTATCGTAA
TTTAAAGCTGCCTCGTATGCTGACCGCCAAGTTCGCTTTACCCAAGCAAG
CTACAGAGCTGAAGGATTTACAGTGTTTAGAGGACGAGCTGGGCCCTCTG
AGGCATGTGCTGGACGGCACCCAGAGCAAGAGCTTCCAGCTGGAGGACGC
CGAGAACTTTATCAGCAACATTCGTGTGACCGTGGTGAAGCTGAAGGGCA
GCGACAACACCTTCGAGTGCCAGTTCGACGACGAGAGCGCCACAGTGGTG
GACTTTTTAAGAAGGTGGATCGCCTTCTGCCAGTCCATCATCAGCACCAG
CCCCCAG (SEQ ID NO:2)

, where the encoded IL-2v polypeptide comprises F76A, Y79A, and L106G substitutions (i.e., comprises Ala-76, Ala-79, and Gly-106). This sequence is codon optimized for expression in mouse.

In some cases, a nucleotide sequence encoding a mouse IL-2v polypeptide is codon optimized for vaccinia virus. The following is a non-limiting example of a nucleotide sequence encoding a mouse IL-2v polypeptide that codon optimized for vaccinia virus:

ATGTACTCGATGCAGTTAGCTTCCTGCGTGACCCTAACCTTAGTCTTGCT
AGTGAATTCGGCGCCCACCTCATCCTCAACGTCATCTTCCACAGCGGAGG
CTCAACAGCAGCAGCAACAGCAGCAACAACAACAGCAGCATTTGGAACAA
TTGCTAATGGACTTACAGGAACTACTATCAAGAATGGAGAATTATCGAAA
CCTAAAGTTACCTCGAATGTTGACAGCAAAATTTGCGTTGCCAAAGCAGG
CCACAGAGCTAAAGGACCTACAGTGTCTTGAAGATGAGCTAGGACCACTT
CGTCACGTTTTAGACGGAACACAGTCCAAGTCTTTTCAGTTGGAAGACGC
CGAGAACTTTATATCTAACATACGTGTTACTGTCGTAAAACTTAAAGGAT
CGGACAATACTTTCGAATGCCAATTCGATGATGAAAGTGCAACCGTCGTG
GACTTCTTGCGACGTTGGATCGCCTTCTGTCAAAGTATAATTTCCACTTC
GCCACAG (SEQ ID NO:19)

.

Suitable amino acid sequences of IL-2v polypeptides include, e.g., a human IL-2v polypeptide comprising an amino acid sequence having at least 95% (e.g., at least 95%, at least 98%, at least 99%, or 100%) amino acid sequence identity to the following amino acid sequence:

MYRMQLLSCIALSLALVTNSAPTSSSTKKTQLQLEHLLLDLQMILNGINN
YKNPKLTRMLTAKFAMPKKATELKHLQCLEEELKPLEEVLNGAQSKNFHL
RPRDLISNINVIVLELKGSETTFMCEYADETATIVEFLNRWITFCQSIIS
TLT (SEQ ID NO: 14)

, and comprising F62A, Y65A, and L92G substitutions (i.e., comprising Ala-62, Ala-65, and Gly-92).

Suitable nucleotide sequences encoding an IL-2v polypeptide include, e.g., a nucleotide sequence encoding a human IL-2v polypeptide and having at least 80%, at least 85%, at least 90%, at least 95%, at least 98%, at least 99%, or 100%, nucleotide sequence identity to the following nucleotide sequence:

ATGTATCGTATGCAGCTGCTGAGCTGCATCGCTTTATCTTTAGCTTTAGT
GACCAACAGCGCCCCTACCAGCTCCTCCACCAAGAAGACCCAGCTGCAGC
TGGAGCATTTACTGCTGGATTTACAGATGATTTTAAACGGCATCAACAAC
TACAAGAACCCCAAGCTGACTCGTATGCTGACCGCCAAGTTCGCTATGCC
CAAGAAGGCCACCGAGCTGAAGCACCTCCAGTGTTTAGAGGAGGAGCTGA
AGCCTTTAGAGGAGGTGCTGAATGGAGCCCAGAGCAAGAATTTCCATTTA
AGGCCTCGTGATTTAATCAGCAACATCAACGTGATCGTGCTGGAGCTGAA
AGGCTCCGAGACCACCTTCATGTGCGAGTACGCCGACGAGACCGCCACCA
TCGTGGAGTTTTTAAATCGTTGGATCACCTTCTGCCAGAGCATCATCAGC
ACTTTAACC (SEQ ID NO: 12)

, where the encoded IL-2v polypeptide comprises F62A, Y65A, and L92G substitutions (i.e., comprises Ala-62, Ala-65, and Gly-92). In some cases, the nucleotide sequence is human codon optimized. SEQ ID NO: 12 is an example of a human codon-optimized IL-2v-encoding nucleotide sequence.

Suitable nucleotide sequences encoding an IL-2v polypeptide include, e.g., a nucleotide sequence encoding a human IL-2v polypeptide and having at least 80%, at least 85%, at least 90%, at least 95%, at least 98%, at least 99%, or 100%, nucleotide sequence identity to the following nucleotide sequence:

ATGTATCGAATGCAATTACTTTCCTGTATCGCACTTTCATTAGCCCTTGT
GACCAACTCAGCGCCAACATCAAGTTCGACCAAGAAGACGCAGTTGCAGC
TAGAGCATTTGCTTTTGGATCTTCAAATGATCCTTAATGGTATAAATAAT
TATAAGAACCCCAAATTGACGCGAATGCTAACAGCTAAATTCGCAATGCC
AAAGAAGGCAACCGAGTTAAAGCACCTACAATGCTTGGAAGAAGAACTAA
AACCCCTTGAGGAGGTATTAAATGGTGCTCAGTCGAAGAATTTTCATCTT
CGACCTCGAGACCTAATTTCAAATATTAACGTAATTGTTTTGGAATTAAA
GGGTTCGGAAACTACTTTTATGTGTGAGTACGCAGACGAGACAGCTACAA
TAGTGGAGTTTCTTAACCGTTGGATAACCTTTTGTCAATCAATCATTTCG
ACTTTGACC (SEQ ID NO: 13)

, where the encoded IL-2v polypeptide comprises F62A, Y65A, and L92G substitutions (i.e., comprises Ala-62, Ala-65, and Gly-92). In some cases, the nucleotide sequence is codon optimized for vaccinia virus. SEQ ID NO: 13 is an example of a vaccinia virus codon-optimized IL-2v-encoding nucleotide sequence.

Suitable amino acid sequences of IL-2v polypeptides include, e.g., a human IL-2v polypeptide comprising an amino acid sequence having at least 95% (e.g., at least 95%, at least 98%, at least 99%, or 100%) amino acid sequence identity to the following amino acid sequence:

APTSSSTKKTQLQLEHLLLDLQMILNGINNYKNPKLTRMLTAKFAMPKK
ATELKHLQCLEEELKPLEEVLNGAQSKNFHLRPRDLISNINVIVLELKG
SETTFMCEYADETATIVEFLNRWITFCQSIISTLT (SEQ ID NO:9)

, and comprising F42A, Y45A, and L72G substitutions (i.e., comprising Ala-42, Ala-45, and Gly-72).

Suitable nucleotide sequences encoding an IL-2v polypeptide include, e.g., a nucleotide sequence encoding a human IL-2v polypeptide and having at least 80%, at least 85%, at least 90%, at least 95%, at least 98%, at least 99%, or 100%, nucleotide sequence identity to the following nucleotide sequence:

GCCCCTACCAGCTCCTCCACCAAGAAGACCCAGCTGCAGCTGGAGCATTT
ACTGCTGGATTTACAGATGATTTTAAACGGCATCAACAACTACAAGAACC
CCAAGCTGACTCGTATGCTGACCGCCAAGTTCGCTATGCCCAAGAAGGCC
ACCGAGCTGAAGCACCTCCAGTGTTTAGAGGAGGAGCTGAAGCCTTTAGA
GGAGGTGCTGAATGGAGCCCAGAGCAAGAATTTCCATTTAAGGCCTCGTG
ATTTAATCAGCAACATCAACGTGATCGTGCTGGAGCTGAAAGGCTCCGAG
ACCACCTTCATGTGCGAGTACGCCGACGAGACCGCCACCATCGTGGAGTT
TTTAAATCGTTGGATCACCTTCTGCCAGAGCATCATCAGCACTTTAACC
(SEQ ID NO: 10)

, where the encoded IL-2v polypeptide comprises F42A, Y45A, and L72G substitutions (i.e., comprises Ala-42, Ala-45, and Gly-72).

In some cases, the nucleotide sequence is human codon optimized. SEQ ID NO: 10 is an example of a human codon-optimized IL-2v-encoding nucleotide sequence.

Suitable nucleotide sequences encoding an IL-2v polypeptide include, e.g., a nucleotide sequence encoding a human IL-2v polypeptide and having at least 80%, at least 85%, at least 90%, at least 95%, at least 98%, at least 99%, or 100%, nucleotide sequence identity to the following nucleotide sequence:

GCGCCAACATCAAGTTCGACCAAGAAGACGCAGTTGCAGCTAGAGCATTT
GCTTTTGGATCTTCAAATGATCCTTAATGGTATAAATAATTATAAGAACC
CCAAATTGACGCGAATGCTAACAGCTAAATTCGCAATGCCAAAGAAGGCA
ACCGAGTTAAAGCACCTACAATGCTTGGAAGAAGAACTAAAACCCCTTGA
GGAGGTATTAAATGGTGCTCAGTCGAAGAATTTTCATCTTCGACCTCGAG
ACCTAATTTCAAATATTAACGTAATTGTTTTGGAATTAAAGGGTTCGGAA
ACTACTTTTATGTGTGAGTACGCAGACGAGACAGCTACAATAGTGGAGTT
TCTTAACCGTTGGATAACCTTTTGTCAATCAATCATTTCGACTTTGACC
(SEQ ID NO:11)

, where the encoded IL-2v polypeptide comprises F42A, Y45A, and L72G substitutions (i.e., comprises Ala-42, Ala-45, and Gly-72).

In some cases, the nucleotide sequence is codon optimized for vaccinia virus. SEQ ID NO: 11 is an example of a vaccinia virus codon-optimized IL-2v-encoding nucleotide sequence.

In some cases, a replication-competent, recombinant oncolytic vaccinia virus of the present disclosure comprises a homologous recombination donor fragment encoding an IL-2v polypeptide, where the homologous recombination donor fragment comprises a nucleotide sequence having at least 80%, at least 85%, at least 90%, at least 95%, at least 98%, at least 99%, or 100%, nucleotide sequence identity to the nucleotide sequence set forth in any one of SEQ ID NO:4 (VV27/VV38 homologous recombination donor fragment), SEQ ID NO:5 (VV39 homologous recombination donor fragment), SEQ ID NO: 15 (VV75 homologous recombination donor fragment containing hIL-2v (human codon optimized)), SEQ ID NO:16 (Copenhagen J2R homologous recombination plasmid containing hIL-2v (human codon optimized)), SEQ ID NO: 17 (homologous recombination donor fragment containing hIL-2v (vaccinia virus codon optimized)), SEQ ID NO:18 (Copenhagen J2R homologous recombination plasmid containing hIL-2v (vaccinia virus codon optimized)), and SEQ ID NO:20 (mouse IL-2 variant (vaccinia virus codon optimized) homologous recombination donor fragment).

In some cases, a replication-competent, recombinant oncolytic vaccinia virus of the present disclosure comprises a nucleotide sequence having at least 80%, at least 85%, at least 90%, at least 95%, at least 98%, at least 99%, or 100%, nucleotide sequence identity to the nucleotide sequence set forth in SEQ ID NO:6 (Copenhagen J2R homologous recombination plasmid); and comprises a nucleic acid comprising a nucleotide sequence encoding an IL-2v polypeptide.

In some cases, a replication-competent, recombinant oncolytic vaccinia virus of the present disclosure comprises a nucleotide sequence having at least 80%, at least 85%, at least 90%, at least 95%, at least 98%, at least 99%, or 100%, nucleotide sequence identity to the nucleotide sequence set forth in SEQ ID NO:7 (Copenhagen J2R homologous recombination plasmid containing mouse IL-2 variant (mIL-2v) polypeptide). In some cases, the replication-competent, recombinant oncolytic vaccinia virus comprises, in place of the mIL-2v polypeptide, a human IL-2 variant (hIL-2v) polypeptide, as described above.

In some cases, a replication-competent, recombinant oncolytic vaccinia virus of the present disclosure comprises a nucleotide sequence having at least 80%, at least 85%, at least 90%, at least 95%, at least 98%, at least 99%, or 100%, nucleotide sequence identity to the nucleotide sequence set forth in SEQ ID NO:8 (Western Reserve J2R homologous recombination plasmid containing mIL-2v). In some cases, the replication-competent, recombinant oncolytic vaccinia virus comprises, in place of the mIL-2v polypeptide, a human IL-2 variant (hIL-2v) polypeptide, as described above.

In some cases, a replication-competent, recombinant oncolytic vaccinia virus of the present disclosure is VV27, (Copenhagen vaccinia containing A34R-K151E and mIL-2v transgene). In some cases, the replication-competent, recombinant oncolytic vaccinia virus comprises, in place of the mIL-2v polypeptide, a human IL-2 variant (hIL-2v) polypeptide, as described above.

In some cases, a replication-competent, recombinant oncolytic vaccinia virus of the present disclosure is VV38, (Copenhagen vaccinia containing mIL-2v transgene). In some cases, the replication-competent, recombinant oncolytic vaccinia virus comprises, in place of the mIL-2v polypeptide, a human IL-2 variant (hIL-2v) polypeptide, as described above.

In some cases, a replication-competent, recombinant oncolytic vaccinia virus of the present disclosure is VV39, (Western Reserve vaccinia containing mIL-2v transgene). In some cases, the replication-competent, recombinant oncolytic vaccinia virus comprises, in place of the mIL-2v polypeptide, a human IL-2 variant (hIL-2v) polypeptide, as described above.

### B-2. Heterologous Thymidine Kinase Polypeptides

As noted above, a replication-competent RVV provided by the present disclosure comprises an inserted nucleotide sequence encoding a heterologous thymidine kinase (TK) polypeptide. In some embodiments, the heterologous TK polypeptide is a human wild-type herpes simplex virus (HSV) TK polypeptide. The amino acid sequence of a human wild-type HSV-TK polypeptide is set forth in SEQ ID NO:25. In some other embodiments, the heterologous TK polypeptide is a variant of wild-type HSV-TK polypeptide. A variant of wild-type HSV-TK is referred to herein as an “HSV-TKv polypeptide,” “TKv polypeptide,” or simply “TKv.” The TKv polypeptide is in some cases a type I TK polypeptide, i.e., a TK polypeptide that can catalyze phosphorylation of deoxyguanosine (dG) to generate dG monophosphate, respectively.

In wild-type vaccinia virus, the J2R region encodes vaccinia virus TK. In some instances, the nucleotide sequence encoding the heterologous TK polypeptide is inserted in the location of the J2R gene of the vaccinia virus. In some other embodiments, the nucleotide sequence encoding the heterologous TK polypeptide replaces all or a part of the vaccinia virus TK-encoding nucleotide sequence. For example, in some cases, the heterologous TK polypeptide-encoding nucleotide sequence replaces at least 10%, at least 15%, at least 20%, at least 25%, at least 30%, at least 40%, at least 50%, at least 75%, or 100%, of the J2R region of vaccinia virus. In some cases, replication-competent, recombinant oncolytic vaccinia virus of the present disclosure comprises a modification such that transcription of the endogenous (vaccinia virus-encoded) TK-encoding gene is reduced or eliminated. For example, in some cases, transcription of the endogenous (vaccinia virus-encoded) TK-encoding gene is reduced by at least 50%, at least 60%, at least 70%, at least 80%, at least 90%, or more than 90%, compared to the transcription of the endogenous (vaccinia virus-encoded) TK-encoding gene without the modification.

In some cases, replication of the replication-competent RVV is inhibited with ganciclovir at a lower concentration than the concentration at which replication of a replication-competent RVV encoding a wild-type HSV-TK polypeptide is inhibited. For example, the ganciclovir inhibitory concentration at which replication of a replication-competent RVV of the present disclosure that encodes a variant of wild-type HSV-TK is inhibited by 50% of maximum (IC50) is at least 10%, at least 15%, at least 20%, at least 25%, at least 30%, at least 40%, at least 50%, at least 60%, at least 70%, or at least 80% lower than the ganciclovir IC50 for inhibition of replication of a replication-competent RVV encoding a wild-type HSV-TK polypeptide.

In some embodiments the heterologous TK polypeptide encoded by a nucleotide sequence present in a replication-competent RVV of the present disclosure is a variant of wild-type HSV-TK, where the TKv polypeptide comprises one or more amino acid substitutions relative to wild-type HSV-TK (SEQ ID NO:25). Thus, e.g., a TKv polypeptide encoded by a nucleotide sequence present in a replication-competent, recombinant oncolytic vaccinia virus of the present disclosure comprises from 1 to 40 amino acid substitutions relative to wild-type HSV-TK. For example, a TKv polypeptide encoded by a nucleotide sequence present in a replication-competent, recombinant oncolytic vaccinia virus of the present disclosure comprises from 1 to 5, from 5 to 10, from 10 to 15, from 15 to 20, from 20 to 25, from 25 to 30, from 30 to 35, or from 35 to 40, amino acid substitutions relative to wild-type HSV-TK (SEQ ID NO:25).

In some embodiments, a heterologous TK polypeptide present in an RVV of the present disclosure comprises an amino acid sequence having at least 80%, at least 85%, at least 90%, at least 95%, at least 98%, or at least 99%, amino acid sequence identity to the following wild-type HSV-TK amino acid sequence:

MASYPGHQHASAFDQAARSRGHSNRRTALRPRRQQEATEVRPEQKMPTLL
RVYIDGPHGMGKTTTTQLLVALGSRDDIVYVPEPMTYWRVLGASETIANI
YTTQHRLDQGEISAGDAAVVMTSAQITMGMPYAVTDAVLAPHIGGEAGSS
HAPPPALTLIFDRHPIAALLCYPAARYLMGSMTPQAVLAFVALIPPTLPG
TNIVLGALPEDRHIDRLAKRQRPGERLDLAMLAAIRRVYGLLANTVRYLQ
GGGSWREDWGQLSGTAVPPQGAEPQSNAGPRPHIGDTLFTLFRAPELLAP
NGDLYNVFAWALDVLAKRLRPMHVFILDYDQSPAGCRDALLQLTSGMIQT
HVTTPGSIPTICDLARTFAREMGEAN (SEQ ID NO:25)

, where the TKv polypeptide comprises one or more amino acid substitutions relative to SEQ ID NO:25.

In some cases, the heterologous TK polypeptide comprises one or more amino acid substitutions relative to the wild-type HSV-TK amino acid sequence (set forth above; SEQ ID NO:25). For example, in some cases, the heterologous TK polypeptide comprises a substitution of one or more of L159, I160, F161, A168, and L169.

In some cases, the heterologous TK polypeptide comprises an amino acid sequence having at least 80%, at least 85%, at least 90%, at least 95%, at least 98%, or at least 99%, amino acid sequence identity to the wild-type HSV-TK amino acid sequence set forth above; SEQ ID NO:25), but has a substitution at L159, i.e., amino acid 159 is other than Leu. For example, amino acid 159 is Gly, Ala, Val, Ile, Pro, Phe, Tyr, Trp, Ser, Thr, Cys, Met, Gln, Asn, Lys, Arg, His, Asp, or Glu. In some cases, the substitution is an L159I substitution. In some cases, the substitution is an L159A substitution. In some cases, the substitution is an L159V substitution.

In some cases, the heterologous TK polypeptide comprises an amino acid sequence having at least 80%, at least 85%, at least 90%, at least 95%, at least 98%, or at least 99%, amino acid sequence identity to the wild-type HSV-TK amino acid sequence set forth above (SEQ ID NO:25), but has a substitution at I160, i.e., amino acid 160 is other than Ile. For example, amino acid 160 is Gly, Ala, Val, Leu, Pro, Phe, Tyr, Trp, Ser, Thr, Cys, Met, Gln, Asn, Lys, Arg, His, Asp, or Glu. In some cases, the substitution is an I160L substitution. In some cases, the substitution is an I160V substitution. In some cases, the substitution is an I160A substitution. In some cases, the substitution is an I160F substitution. In some cases, the substitution is an I160Y substitution. In some cases, the substitution is an I160W substitution.

In some cases, the heterologous TK polypeptide comprises an amino acid sequence having at least 80%, at least 85%, at least 90%, at least 95%, at least 98%, or at least 99%, amino acid sequence identity to the wild-type HSV-TK amino acid sequence set forth above (SEQ ID NO:25), but has a substitution at F161, i.e., amino acid 161 is other than Phe. For example, amino acid 161 is Gly, Ala, Val, Leu, Ile, Pro, Tyr, Trp, Ser, Thr, Cys, Met, Gln, Asn, Lys, Arg, His, Asp, or Glu. In some cases, the substitution is an F161A substitution. In some cases, the substitution is an F161L substitution. In some cases, the substitution is an F161V substitution. In some cases, the substitution is an F161I substitution.

In some cases, the heterologous TK polypeptide comprises an amino acid sequence having at least 80%, at least 85%, at least 90%, at least 95%, at least 98%, or at least 99%, amino acid sequence identity to the wild-type HSV-TK amino acid sequence set forth above (SEQ ID NO:25), but has a substitution at A168, i.e., amino acid 168 is other than Ala. For example, amino acid 168 is Gly, Val, Leu, Ile, Pro, Phe, Tyr, Trp, Ser, Thr, Cys, Met, Gln, Asn, Lys, Arg, His, Asp, or Glu. In some cases, the substitution is A168H. In some cases, the substitution is A168R. In some cases, the substitution is A168K. In some cases, the substitution is A168Y. In some cases, the substitution is A168F. In some cases, the substitution is A168W. In some cases, the TKv polypeptide does not include any other substitutions other than a substitution of A168.

In some cases, the heterologous TK polypeptide comprises an amino acid sequence having at least 80%, at least 85%, at least 90%, at least 95%, at least 98%, or at least 99%, amino acid sequence identity to the wild-type HSV-TK amino acid sequence set forth above (SEQ ID NO:25), but has a substitution at L169, i.e., amino acid 169 is other than Leu. For example, amino acid 169 is Gly, Ala, Val, Ile, Pro, Phe, Tyr, Trp, Ser, Thr, Cys, Met, Gln, Asn, Lys, Arg, His, Asp, or Glu. In some cases, the substitution is L169F. In some cases, the substitution is L169M. In some cases, the substitution is L169Y. In some cases, the substitution is L169W.

In some cases, the heterologous TK polypeptide comprises an amino acid sequence having at least 80%, at least 85%, at least 90%, at least 95%, at least 98%, or at least 99%, amino acid sequence identity to the wild-type HSV-TK amino acid sequence set forth above (SEQ ID NO:25), where: i) amino acid 159 is other than Leu; ii) amino acid 160 is other than Ile; iii) amino acid 161 is other than Phe; iv) amino acid 168 is other than Ala; and v) amino acid 169 is other than Leu. In some cases, the heterologous TK polypeptide comprises an amino acid sequence having at least 80%, at least 85%, at least 90%, at least 95%, at least 98%, at least 99%, or 100%, amino acid sequence identity to the following amino acid sequence:

MASYPGHQHASAFDQAARSRGHSNRRTALRPRRQQEATEVRPEQKMPTLL
RVYIDGPHGMGKTTTTQLLVALGSRDDIVYVPEPMTYWRVLGASETIANI
YTTQHRLDQGEISAGDAAVVMTSAQITMGMPYAVTDAVLAPHIGGEAGSS
HVPPPALTILADRHPIAYFLCYPAARYLMGSMTPQAVLAFVALIPPTLPG
TNIVLGALPEDRHIDRLAKRQRPGERLDLAMLAAIRRVYGLLANTVRYLQ
GGGSWREDWGQLSGTAVPPQGAEPQSNAGPRPHIGDTLFTLFRAPELLAP
NGDLYNVFAWALDVLAKRLRPMHVFILDYDQSPAGCRDALLQLTSGMIQT
HVTTPGSIPTICDLARTFAREMGEAN (“dm30”; SEQ ID NO:26)

, where amino acid 159 is Ile, amino acid 160 is Leu, amino acid 161 is Ala, amino acid 168 is Tyr, and amino acid 169 is Phe.

In some cases, the heterologous TK polypeptide comprises an amino acid sequence having at least 80%, at least 85%, at least 90%, at least 95%, at least 98%, or at least 99%, amino acid sequence identity to the wild-type HSV-TK amino acid sequence set forth above (SEQ ID NO:25), where: i) amino acid 159 is other than Leu; ii) amino acid 160 is other than Ile; iii) amino acid 161 is other than Phe; iv) amino acid 168 is other than Ala; and v) amino acid 169 is other than Leu. In some cases, the heterologous TK polypeptide comprises an amino acid sequence having at least 80%, at least 85%, at least 90%, at least 95%, at least 98%, at least 99%, or 100%, amino acid sequence identity to the following amino acid sequence:

MASYPGHQHASAFDQAARSRGHSNRRTALRPRRQQEATEVRPEQKMPTLL
RVYIDGPHGMGKTTTTQLLVALGSRDDIVYVPEPMTYWRVLGASETIANI
YTTQHRLDQGEISAGDAAVVMTSAQITMGMPYAVTDAVLAPHIGGEAGSS
HAPPPALTIFLDRHPIAFMLCYPAARYLMGSMTPQAVLAFVALIPPTLPG
TNIVLGALPEDRHIDRLAKRQRPGERLDLAMLAAIRRVYGLLANTVRYLQ
GGGSWREDWGQLSGTAVPPQGAEPQSNAGPRPHIGDTLFTLFRAPELLAP
NGDLYNVFAWALDVLAKRLRPMHVFILDYDQSPAGCRDALLQLTSGMIQT
HVTTPGSIPTICDLARTFAREMGEAN (“SR39”; SEQ ID NO:27)

, where amino acid 159 is Ile, amino acid 160 is Phe, amino acid 161 is Leu, amino acid 168 is Phe, and amino acid 169 is Met.

In some cases, the heterologous TK polypeptide comprises an amino acid sequence having at least 80%, at least 85%, at least 90%, at least 95%, at least 98%, or at least 99%, amino acid sequence identity to the wild-type HSV-TK amino acid sequence set forth above (SEQ ID NO:25), where amino acid 168 is other than Ala, e.g., where amino acid 168 is Gly, Val, Ile, Leu, Pro, Phe, Tyr, Trp, Ser, Thr, Cys, Met, Gln, Asn, Lys, Arg, His, Asp, or Glu. In some cases, amino acid 168 is His. In some cases, amino acid 168 is Arg. In some cases, amino acid 168 is Lys. In some cases, the heterologous TK polypeptide comprises an amino acid sequence having at least 80%, at least 85%, at least 90%, at least 95%, at least 98%, at least 99%, or 100%, amino acid sequence identity to the following amino acid sequence:

MASYPGHQHASAFDQAARSRGHSNRRTALRPRRQQEATEVRPEQKMPTLL
RVYIDGPHGMGKTTTTQLLVALGSRDDIVYVPEPMTYWRVLGASETIANI
YTTQHRLDQGEISAGDAAVVMTSAQITMGMPYAVTDAVLAPHIGGEAGSS
HAPPPALTLIFDRHPIAHLLCYPAARYLMGSMTPQAVLAFVALIPPTLPG
TNIVLGALPEDRHIDRLAKRQRPGERLDLAMLAAIRRVYGLLANTVRYLQ
GGGSWREDWGQLSGTAVPPQGAEPQSNAGPRPHIGDTLFTLFRAPELLAP
NGDLYNVFAWALDVLAKRLRPMHVFILDYDQSPAGCRDALLQLTSGMIQT
HVTTPGSIPTICDLARTFAREMGEAN (“TK.007”; SEQ ID NO:28
)

, where amino acid 168 is His.

The heterologous TK polypeptide of SEQ ID NO:28 where amino acid 168 is His is also referred to as “TK.007” or HSV-TK.007” in the present disclosure.

### B-3. Other Modifications

In addition to an inserted nucleotide sequence encoding an immunostimulatory cytokines and an inserted nucleotide sequence encoding a heterologous TK, an RVV provided by the present disclosure may comprise further modifications in the viral genome or viral proteins relative to the parent virus to further improve the properties of the recombinant oncolytic vaccinia virus, such as modifications that increase or enhance its desirable properties as an oncolytic virus, such as modifications to render deficient the function of a specific protein, to suppress or enhance the expression of a specific gene or protein, or to express an exogenous protein.

In some embodiments, the RVV provided by the present disclosure further comprises one or more modifications that increase the tumor-selectivity of the oncolytic vaccinia viruses. As used herein, “tumor selective” means toxicity to tumor cells (for example, oncolytic) higher than that to normal cells (for example, non-tumor cell). Examples of such modifications include: (1) modification that renders the virus deficient in the function of vaccinia growth factor (VGF) (McCart et al. (2001) Cancer Research 61:8751); (2) modification to the vaccinia virus TK gene to render the virus TK deficient, or modifications to the hemagglutinin (HA) gene, or F3 gene or an interrupted F3 locus (WO 2005/047458); (3) modification that renders the vaccinia virus deficient in the function of VGF and O1L (WO 2015/076422); (4) insertion of a micro RNA whose expression is decreased in cancer cells into the 3′ noncoding region of the B5R gene (WO 2011/125469); (5) modifications that render the vaccinia virus deficient in the function of B18R (Kirn et al. (2007) PLoS Medicine 4:e353), ribonucleotide reductase (Gammon et al. (2010) PLoS Pathogens 6:e1000984), serine protease inhibitor (e.g., SPI-1, SPI-2) (Guo et al. (2005) Cancer Research 65:9991), SPI-1 and SPI-2 (Yang et al. (2007) Gene Therapy 14:638), ribonucleotide reductase genes F4L or I4L (Child et al. (1990) Virology 174:625; Potts et al. (2017) EMBO Mol. Med. 9:638), B18R (B19R in Copenhagen strain) (Symons et al. (1995) Cell 81:551), A48R (Hughes et al. (1991) J. Biol. Chem. 266:20103); B8R (Verardi et al. (2001) J. Virol. 75:11), B15R (B16R in Copenhagen strain) (Spriggs et al. (1992) Cell 71:145), A41R (Ng et al. (2001) Journal of General Virology 82:2095), A52R (Bowie et al. (2000) Proc. Natl. Acad. Sci. USA 97:10162), F1L (Gerlic et al. (2013) Proc. Natl. Acad. Sci. USA 110:7808), E3L (Chang et al. (1992) Proc. Natl. Acad. Sci. USA 89:4825), A44R-A46R (Bowie et al. (2000) Proc. Natl. Acad. Sci. USA 97:10162), K1L (Bravo Cruz et al. (2017) Journal of Virology 91:e00524), A48R, B18R, C11R, and TK (Mejías-Pérez et al. (2017) Molecular Therapy: Oncolytics 8:27), E3L and K3L regions (WO 2005/007824), or O1L (Schweneker et al. (2012) J. Virol. 86:2323). Moreover, an RVV may comprise a modification that renders the vaccinia virus deficient in the extracellular region of B5R (Bell et al. (2004) Virology 325:425), deficient in the A34R region (Thirunavukarasu et al. (2013) Molecular Therapy 21:1024), or deficient in interleukin-1□ (IL-1□) receptor (WO 2005/030971). Moreover, vaccinia virus having a combination of two or more of such genetic modifications may be used in a replication-competent, recombinant oncolytic vaccinia virus of the present disclosure. Such insertion of a foreign gene or deletion or mutation of a gene on the vaccinia virus genome can be made, for example, by a known homologous recombination or site-directed mutagenesis.

As used herein, the term “deficient” or “deficiency” means that the gene region or protein specified by this term has reduced or no function. A gene or protein can be rendered deficient by ways known in the art, such as: i) mutation (e.g., substitution, inversion, etc.) and/or truncation and/or deletion of the gene region specified by this term; ii) mutation and/or truncation and/or deletion of a promoter region controlling expression of the gene region; and iii) mutation and/or truncation and/or deletion of a polyadenylation sequence such that translation of a polypeptide encoded by the gene region is reduced or eliminated. A replication-competent RVV of the present disclosure that comprises a modification such that the virus is rendered “deficient” in a given vaccinia virus gene exhibits reduced production and/or activity of a gene product (e.g., mRNA gene product; polypeptide gene product) of the gene; for example, the amount and/or activity of the gene product is less than 75%, less than 60%, less than 50%, less than 40%, less than 30%, less than 25%, less than 20%, less than 15%, less than 10%, less than 5%, or less than 1% of the amount and/or activity of the same gene product produced by wild-type vaccinia virus, or by a control vaccinia virus that does not comprise the genetic alteration. For example, being ‘deficient” may be a result of the deletion in a region consisting of the specified gene region or the deletion in a neighboring gene region comprising the specified gene region. As an example, a mutation and/or truncation and/or deletion of a promoter region that reduces transcription of a gene region can result in deficiency. A gene region can also be rendered deficient through incorporation of a transcriptional termination element such that translation of a polypeptide encoded by the gene region is reduced or eliminated. A gene region can also be rendered deficient through use of a gene-editing enzyme or a gene-editing complex (e.g., a CRISPR/Cas effector polypeptide complexed with a guide RNA) to reduce or eliminate transcription of the gene region. A gene region can also be rendered deficient through use of competitive reverse promoter/polymerase occupancy to reduce or eliminate transcription of the gene region. A gene region can also be rendered deficient by insertion of a nucleic acid into the gene region, thereby knocking out the gene region.

In some specific embodiments, an RVV of the present disclosure lacks the vaccinia virus’s endogenous thymidine kinase (TK) activity. As used herein, the term “endogenous” refers to any materials, such as polynucleotide, polypeptide, or protein, that is naturally present or naturally expressed within an organism, such as a virus, or a cell thereof. The vaccinia virus TK is encoded by the TK gene and open-reading frame (ORF) J2R on the vaccinia virus genome. A virus that lacks endogenous TK activity may be referred to as being “thymidine kinase negative,” “TK negative,” “thymidine kinase deficient,” or “TK deficient.” In some cases, an RVV of the present disclosure comprises a deletion of all or a portion of the vaccinia virus TK coding region, such that the vaccinia virus is TK deficient. For example, in some cases, a replication-competent, recombinant oncolytic vaccinia virus of the present disclosure comprises a J2R deletion. See, e.g., Mejía-Perez et al. (2018) Mol. Ther. Oncolytics 8:27. In some cases, a replication-competent, recombinant oncolytic vaccinia virus of the present disclosure comprises an insertion into the J2R region, thereby resulting in reduced or no vaccinia virus TK activity.

In some embodiments, the present disclosure provides an RVV wherein the A34R gene of the virus comprises a K151E substitution (i.e., comprising a modification that provides for a K151E substitution in the encoded polypeptide). See, e.g., Blasco et al. (1993) J. Virol. 67(6):3319-3325; and Thirunavukarasu et al. (2013) Mol. Ther. 21:1024. The A34R gene encodes vaccinia virus gp22-24 (also known as Protein A34). The amino acid sequence of an A34 protein of the vaccinia virus strain Copenhagen is available at UniProt (UniProtKB-P21057 (Q34_VACCC)), which consists of 168 amino acids.

In some embodiments, the RVV provided by the present disclosure comprises: (1) an inserted nucleotide sequence encoding an IL-2v polypeptide; (2) an inserted nucleotide sequence encoding a heterologous TK polypeptide; and (3) a K151E substitution in the A34R gene, wherein the RVV is TK deficient. In some particular embodiments, the IL-2v polypeptide encoded by the RVV comprises an amino acid sequence having at least 95% (e.g., at least 95%, at least 98%, at least 99%, or 100%) identity to the amino acid sequence of in SEQ ID NO:1 and comprises an amino acid substitution an F42A substitution, a Y45A substitution, and an L72G substitution, wherein the amino acid numbering is based on the amino acid sequence of SEQ ID NO:1. In some further particular embodiments, the heterologous TK polypeptide comprises an amino acid sequence having at least 95% (e.g., at least 95%, at least 98%, at least 99%, or 100%) identity to the amino acid sequence of in SEQ ID NO:28 where amino acid 168 is His.

### B-4. Construction of Recombinant Vaccinia Virus

A replication-competent, recombinant oncolytic vaccinia virus of the present disclosure can be constructed from any of a variety of strains of vaccinia virus, either known now or discovered in the future. Strains of the vaccinia virus suitable for use include, but not limited to, the strains Lister, New York City Board of Health (NYBH), Wyeth, Copenhagen, Western Reserve (WR), Modified Vaccinia Ankara (MVA), EM63, Ikeda, Dalian, LIVP, Tian Tan, IHD-J, Tashkent, Bern, Paris, Dairen, and derivatives the like. In some cases, a replication-competent RVV of the present disclosure is a Copenhagen strain vaccinia virus. In some cases, a replication-competent RVV of the present disclosure is a WR strain vaccinia virus.

The nucleotide sequences of the genomes of vaccinia viruses of various strains are known in the art. See, e.g., Goebel et al. (1990) Virology 179:247; Goebel et al. (1990) Virology 179:517. The nucleotide sequence of the Copenhagen strain vaccinia virus is known; see, e.g., GenBank Accession No. M35027. The nucleotide sequence of the WR strain vaccinia virus is known; see, e.g., GenBank Accession No. AY243312; and GenBank Accession No. NC_006998. The WR strain of vaccinia virus is available from the American Type Culture Collection (ATCC); ATCC VR-1354. In a particular embodiment, an RVV provided by the present disclosure comprises: (1) an inserted nucleotide sequence encoding an IL-2v polypeptide; (2) an inserted nucleotide sequence encoding a heterologous TK polypeptide; and (3) a K151E substitution in the A34R gene, wherein the RVV is Strain Copenhagen and is TK deficient, wherein the IL-2v polypeptide comprises the amino acid sequence SEQ ID NO:1 and comprises an amino acid substitution an F42A substitution, a Y45A substitution, and an L72G substitution, and wherein the heterologous TK polypeptide comprises an amino acid sequence of SEQ ID NO:28 where amino acid 168 is His.

A replication-competent RVV of the present disclosure exhibits oncolytic activity. The oncolytic activity of a virus can be evaluated by any suitable method known in the art. Examples of methods for evaluating whether a given virus exhibits oncolytic activity include in vitro methods for evaluating decrease of the survival rate of cancer cells by the addition of the virus. Examples of cancer cells or cell lines that may be used include the malignant melanoma cell RPMI-7951 (for example, ATCC HTB-66), the lung adenocarcinoma HCC4006 (for example, ATCC CRL-2871), the lung carcinoma A549 (for example, ATCC CCL-185), the lung carcinoma HOP-62 (for example, DCTD Tumor Repository), the lung carcinoma EKVX (for example, DCTD Tumor Repository), the small cell lung cancer cell DMS 53 (for example, ATCC CRL-2062), the lung squamous cell carcinoma NCI-H226 (for example, ATCC CRL-5826), the kidney cancer cell Caki-1 (for example, ATCC HTB-46), the bladder cancer cell 647-V (for example, DSMZ ACC 414), the head and neck cancer cell Detroit 562 (for example, ATCC CCL-138), the breast cancer cell JIMT-1 (for example, DSMZ ACC 589), the breast cancer cell MDA-MB-231 (for example, ATCC HTB-26), the breast cancer cell MCF7 (for example, ATCC HTB-22), the breast cancer HS-578T (for example, ATCC HTB-126), the breast ductal carcinoma T-47D (for example, ATCC HTB-133), the esophageal cancer cell OE33 (for example, ECACC 96070808), the glioblastoma U-87MG (for example, ECACC 89081402), the neuroblastoma GOTO (for example, JCRB JCRB0612), the myeloma RPMI 8226 (for example, ATCC CCL-155), the ovarian cancer cell SK-OV-3 (for example, ATCC HTB-77), the ovarian cancer cell OVMANA (for example, JCRB JCRB1045), the cervical cancer HeLa (for example, ATCC CCL-2), the colon cancer cell RKO (for example, ATCC CRL-2577), the colon cancer cell HT-29 (for example, ATCC HTB-38), the colon cancer Colo 205 (for example, ATCC CCL-222), the colon cancer SW620 (for example, ATCC CCL-227), the colorectal carcinoma HCT 116 (for example, ATCC CCL-247), the pancreatic cancer cell BxPC-3 (for example, ATCC CRL-1687), the bone osteosarcoma U-2 OS (for example, ATCC HTB-96), the prostate cancer cell LNCaP clone FGC (for example, ATCC CRL-1740), the hepatocellular carcinoma JHH-4 (for example, JCRB JCRB0435), the mesothelioma NCI-H28 (for example, ATCC CRL-5820), the cervical cancer cell SiHa (for example, ATCC HTB-35), and the gastric cancer cell Kato III (for example, RIKEN BRC RCB2088).

A nucleic acid comprising a nucleotide sequence encoding an IL-2v polypeptide or heterologous TK polypeptide can be introduced into a vaccinia virus using established techniques. An example of a suitable technique is reactivation with helper virus. Another example of a suitable technique is as homologous recombination. For example, a plasmid (also referred to as transfer vector plasmid DNA) in which a nucleic acid comprising a nucleotide sequence encoding an IL-2v polypeptide is inserted can be generated, generating a recombinant transfer vector; the recombinant transfer vector can be introduced into cells infected with vaccinia virus. The nucleic acid comprising a nucleotide sequence encoding the IL-2v polypeptide is then introduced into the vaccinia virus from the recombinant transfer vector via homologous recombination. The region in which a nucleic acid comprising a nucleotide sequence encoding an IL-2v polypeptide is introduced can be a gene region that is inessential for the life cycle of vaccinia virus. For example, the region in which a nucleic acid comprising a nucleotide sequence encoding an IL-2v polypeptide is introduced can be a region within the VGF gene in vaccinia virus deficient in the VGF function, a region within the O1L gene in vaccinia virus deficient in the O1L function, or a region or regions within either or both of the VGF and O1L genes in vaccinia virus deficient in both VGF and O1L functions. In the above, the foreign gene(s) can be introduced so as to be transcribed in the direction same as or opposite to that of the VGF and O1L genes. As another example, the region in which a nucleic acid comprising a nucleotide sequence encoding an IL-2v polypeptide is introduced can be a region within the B18 gene (B19 in Copenhagen) in vaccinia virus deficient in B18 (B19) function.

Similarly, a plasmid (also referred to as transfer vector plasmid DNA) in which a nucleotide sequence encoding a heterologous TK polypeptide is inserted can be generated, generating a recombinant transfer vector; the recombinant transfer vector can be introduced into cells transfected with digested genomic DNA from Vaccinia virus and infected with a helper virus. The nucleotide sequence encoding the TKv polypeptide is then introduced into the vaccinia virus from the recombinant transfer vector via homologous recombination. The region in which a nucleotide sequence encoding a TKv polypeptide is introduced can be the endogenous vaccinia virus TK-encoding gene, e.g., J2R. The nucleic acid encoding a TKv polypeptide can replace all or a portion of vaccinia virus J2R.

In some case, the nucleotide sequence encoding the IL-2v polypeptide or heterologous TK polypeptide is operably linked to a transcriptional control element, e.g., a promoter. In some cases, the promoter provides for expression of the polypeptide in tumor cells. Suitable promoters include, but are not limited to, a pSEL promoter, a PSFJ1-10 promoter, a PSFJ2-16 promoter, a pHyb promoter, a Late-Early optimized promoter, a p7.5K promoter, a p11K promoter, a T7.10 promoter, a CPX promoter, a modified H5 promoter, an H4 promoter, a HF promoter, an H6 promoter, and a T7 hybrid promoter.

In some cases, the nucleotide sequence encoding the IL-2v polypeptide or heterologous TK polypeptide is operably linked to a regulatable promoter. In some cases, the regulatable promoter is a reversible promoter. In some cases, the nucleotide sequence encoding the IL-2v polypeptide or heterologous TK polypeptide is operably linked to a tetracycline-regulated promoter, (e.g., a promoter system such as TetActivators, TetON, TetOFF, Tet-On Advanced, Tet-On 3G, etc.). In some cases, the nucleotide sequence encoding the IL-2v polypeptide or heterologous TK polypeptide is operably linked to a repressible promoter. In some cases, the nucleotide sequence encoding the IL-2v polypeptide or heterologous TK polypeptide is operably linked to a promoter that is tetracycline repressible, e.g., the promoter is repressed in the presence of tetracycline or a tetracycline analog or derivative. In some cases, the nucleotide sequence encoding the IL-2v polypeptide or heterologous TK polypeptide is operably linked to a TetOFF promoter system. Bujard and Gossen (1992) Proc. Natl. Acad. Sci. USA 89:5547. For example, a TetOFF promoter system is repressed (inactive) in the presence of tetracycline (or suitable analog or derivative, such as doxycycline); once tetracycline is removed, the promoter is active and drives expression of the polypeptide. In some cases, the nucleotide sequence encoding the IL-2v polypeptide or heterologous TK polypeptide is operably linked to a promoter that is tetracycline activatable, e.g., the promoter is activated in the presence of tetracycline or a tetracycline analog or derivative.

### C. Compositions

In another aspect, the present disclosure provides a composition comprising an RVV provided by the present disclosure. In some cases, the composition is a pharmaceutical composition. In some cases, the pharmaceutical composition is suitable for administering to human in need thereof.

A pharmaceutical composition provided by the present disclosure may further include a pharmaceutically acceptable carrier(s). As used herein, the term “pharmacologically acceptable carrier” refers to any substance that has substantially no long-term or permanent detrimental effect when administered and encompasses terms such as pharmacologically acceptable “vehicle,” “stabilizer,” “diluent,” “auxiliary” or “excipient.” Such a carrier generally is mixed with an RVV of the present disclosure, and can be a solid, semi-solid, or liquid agent. It is understood that an RVV of the present disclosure can be soluble or can be delivered as a suspension in the desired carrier or diluent. Any of a variety of pharmaceutically acceptable carriers can be used including, without limitation, buffers, preservatives, tonicity adjusters, salts, antioxidants, bulking agents, emulsifying agents, wetting agents, and the like. Various buffers and means for adjusting pH can be used to prepare a pharmaceutical composition disclosed in the present specification, provided that the resulting preparation is pharmaceutically acceptable. Such buffers include, without limitation, acetate buffers, citrate buffers, phosphate buffers, neutral buffered saline, phosphate buffered saline and borate buffers. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole and butylated hydroxytoluene. Useful preservatives include, without limitation, benzalkonium chloride, chlorobutanol, thimerosal, phenylmercuric acetate, phenylmercuric nitrate and a stabilized oxy chloro composition, for example, PURITE™. Tonicity adjustors suitable for inclusion in a subject pharmaceutical composition include, without limitation, salts such as, e.g., sodium chloride, potassium chloride, mannitol or glycerin and other pharmaceutically acceptable tonicity adjustor. It is understood that these and other substances known in the art of pharmacology can be included in a subject pharmaceutical composition.

A pharmaceutical composition of the present disclosure can comprise an RVV of the present disclosure in an amount of from about 10² plaque-forming units (pfu) per ml (pfu/ml) to about 10⁴ pfu/ml, from about 10⁴ pfu/ml to about 10⁵ pfu/ml, from about 10⁵ pfu/ml to about 10⁶ pfu/ml, from about 10⁶ pfu/ml to about 10⁷ pfu/ml, from about 10⁷ pfu/ml to about 10⁸ pfu/ml, from about 10⁸ pfu/ml to about 10⁹ pfu/ml, from about 10⁹ pfu/ml to about 10¹⁰ pfu/ml, from about 10¹⁰ pfu/ml to about 10¹¹ pfu/ml, or from about 10¹¹ pfu/ml to about 10¹² pfu/ml.

### D. Uses and Methods of Inducing Oncolysis and Treatment of Cancer

### D-1. Method, Use, and Administration

In another aspect, the present disclosure provides uses of, as well as method of using, the recombinant oncolytic vaccinia viruses and compositions comprising the recombinant oncolytic vaccinia virus. The uses or methods includes those for inducing oncolysis, or treating cancer, in an individual having a tumor, the methods comprising administering to the individual in need thereof an effective amount of a replication-competent RVV of the present disclosure or a composition of the present disclosure. Administration of an effective amount of a replication-competent RVV of the present disclosure, or a composition of the present disclosure, is also referred to herein as “virotherapy.”

In some cases, an “effective amount” of a replication-competent RVV of the present disclosure is an amount that, when administered in one or more doses to an individual in need thereof, reduces the number of cancer cells or tumor mass in the individual. For example, in some cases, an “effective amount” of a replication-competent, RVV is an amount that, when administered in one or more doses to an individual in need thereof, reduces the number of cancer cells in the individual by at least 10%, at least 15%, at least 20%, at least 25%, at least 30%, at least 40%, at least 50%, at least 60%, at least 70%, at least 80%, at least 90%, or at least 95%, compared to the number of cancer cells in the individual before administration of the RVV, or in the absence of administration with the RVV. In some cases, an “effective amount” of an RVV is an amount that, when administered in one or more doses to an individual in need thereof, reduces the number of cancer cells in the individual to undetectable levels. In some cases, an “effective amount” of an RVV of the present disclosure is an amount that, when administered in one or more doses to an individual in need thereof, reduces the tumor mass in the individual. For example, in some cases, an “effective amount” of an RVV of the present disclosure is an amount that, when administered in one or more doses to an individual in need thereof, reduces the tumor mass in the individual by at least 10%, at least 15%, at least 20%, at least 25%, at least 30%, at least 40%, at least 50%, at least 60%, at least 70%, at least 80%, at least 90%, or at least 95%, compared to the tumor mass in the individual before administration of the RVV, or in the absence of administration with the replication-competent, recombinant oncolytic vaccinia virus.

In some cases, an “effective amount” of an RVV of the present disclosure is an amount that, when administered in one or more doses to an individual in need thereof, increases survival time of the individual. For example, in some cases, an “effective amount” of an RVV of the present disclosure is an amount that, when administered in one or more doses to an individual in need thereof, increases survival time of the individual by at least 1 month, at least 2 months, at least 3 months, from 3 months to 6 months, from 6 months to 1 year, from 1 year to 2 years, from 2 years to 5 years, from 5 years to 10 years, or more than 10 years, compared to the expected survival time of the individual in the absence of administration with the replication-competent, recombinant oncolytic vaccinia virus.

In some cases, an “effective amount” of an RVV of the present disclosure is an amount that, when administered in one or more doses to an individual in need thereof, provides for an increase in the number of IFN-γ-producing T cells. For example, in some cases, an “effective amount” of an RVV of the present disclosure is an amount that, when administered in one or more doses to an individual in need thereof, provides for an increase in the number of IFN-γ-producing T cells in the individual of at least 10%, at least 25%, at least 50%, at least 2-fold, at least 5-fold, or at least 10-fold, compared to the number of IFN-γ-producing T cells in the individual before administration of the replication-competent, recombinant oncolytic vaccinia virus, or in the absence of administration with the replication-competent, recombinant oncolytic vaccinia virus.

In some cases, an “effective amount” of an RVV of the present disclosure is an amount that, when administered in one or more doses to an individual in need thereof, provides for an increase in the circulating level of IL-2 or IL-2v in the individual. For example, in some cases, an “effective amount” of an RVV of the present disclosure is an amount that, when administered in one or more doses to an individual in need thereof, provides for an increase in the circulating level of IL-2 or IL-2v in the individual at least 10%, at least 25%, at least 50%, at least 2-fold, at least 2-fold, or at least 10-fold, compared to the circulating level of IL-2 or IL-2v in the individual before administration of the replication-competent, recombinant oncolytic vaccinia virus, or in the absence of administration with the replication-competent, recombinant oncolytic vaccinia virus.

In some cases, an “effective amount” of an RVV of the present disclosure is an amount that, when administered in one or more doses to an individual in need thereof, provides for an increase in the circulating level of IL-2v polypeptide in the individual. For example, in some cases, an “effective amount” of an RVV of the present disclosure is an amount that, when administered in one or more doses to an individual in need thereof, provides for an increase in the circulating level of IL-2v polypeptide in the individual at least 10%, at least 25%, at least 50%, at least 2-fold, at least 5-fold, or at least 10-fold, compared to the circulating level of IL-2v polypeptide in the individual before administration of the replication-competent, recombinant oncolytic vaccinia virus, or in the absence of administration with the replication-competent, recombinant oncolytic vaccinia virus.

In some cases, an “effective amount” of an RVV of the present disclosure is an amount that, when administered in one or more doses to an individual in need thereof, provides for an increase in the number of CD8⁺ tumor-infiltrating lymphocytes (TILs). For example, in some cases, an “effective amount” of an RVV of the present disclosure is an amount that, when administered in one or more doses to an individual in need thereof, provides for an increase in the number of CD8⁺ TILs of at least 10%, at least 25%, at least 50%, at least 2-fold, at least 5-fold, or at least 10-fold, compared to the number of CD8⁺ TILs in the individual before administration of the replication-competent, recombinant oncolytic vaccinia virus, or in the absence of administration with the replication-competent, recombinant oncolytic vaccinia virus.

In some cases, an “effective amount” of an RVV of the present disclosure is an amount that, when administered in one or more doses to an individual in need thereof, induces a durable anti-tumor immune response, e.g., an anti-tumor immune response that provides for reduction in tumor cell number and/or tumor mass and/or tumor growth for at least 1 month, at least 2 months, at least 6 months, or at least 1 year.

A suitable dosage can be determined by an attending physician, or other qualified medical personnel, based on various clinical factors. As is well known in the medical arts, dosages for any one patient depend upon many factors, including the patient’s size, body surface area, age, tumor burden, and other relevant factors.

An RVV of the present disclosure can be administered in an amount of from about 10² plaque-forming units (pfu) to about 10⁴ pfu, from about 10⁴ pfu to about 10⁵ pfu, from about 10⁵ pfu to about 10⁶ pfu, from about 10⁶ pfu to about 10⁷ pfu, from about 10⁷ pfu to about 10⁸ pfu, from about 10⁸ pfu to about 10⁹ pfu, from about 10⁹ pfu to about 10¹⁰ pfu, or from about 10¹⁰ pfu to about 10¹¹ pfu, per dose.

In some cases, an RVV of the present disclosure is administered in a total amount of from about 1 × 10⁹ pfu to 5 × 10¹¹ pfu. In some cases, an RVV of the present disclosure is administered in a total amount of from about 1 × 10⁹ pfu to about 5 × 10⁹ pfu, from about 5 × 10⁹ pfu to about 10¹⁰ pfu, from about 10¹⁰ pfu to about 5 × 10¹⁰ pfu, from about 5 × 10¹⁰ pfu to about y, or from about 10¹¹ pfu to about 5 × 10¹¹ pfu. In some cases, an RVV of the present disclosure is administered in a total amount of about 2 × 10¹⁰ pfu.

In some cases, an RVV of the present disclosure is administered in an amount of from about 1 × 10⁸ pfu/kg patient weight to about 5 × 10⁹ pfu/kg patient weight. In some cases, an RVV of the present disclosure is administered in an amount of from about 1 × 10⁸ pfu/kg patient weight to about 5 × 10⁸ pfu/kg patient weight, from about 5 × 10⁸ pfu/kg patient weight to about 10⁹ pfu/kg patient weight, or from about 10⁹ pfu/kg patient weight to about 5 × 10⁹ pfu/kg patient weight. In some cases, an RVV of the present disclosure is administered in an amount of 1 × 10⁸ pfu/kg patient weight. In some cases, an RVV of the present disclosure is administered in an amount of 2 × 10⁸ pfu/kg patient weight. In some cases, an RVV of the present disclosure is administered in an amount of 3 × 10⁸ pfu/kg patient weight. In some cases, an RVV of the present disclosure is administered in an amount of 4 × 10⁸ pfu/kg patient weight. In some cases, an RVV of the present disclosure is administered in an amount of 5 × 10⁸ pfu/kg patient weight.

In some cases, multiple doses of an RVV of the present disclosure are administered. The frequency of administration of an RVV of the present disclosure can vary depending on any of a variety of factors, e.g., severity of the symptoms, etc. For example, in some embodiments, an RVV of the present disclosure is administered once per month, twice per month, three times per month, every other week (qow), once per week (qw), twice per week (biw), three times per week (tiw), four times per week, five times per week, six times per week, every other day (qod), daily (qd), twice a day (bid), or three times a day (tid).

The duration of administration of an RVV of the present disclosure, e.g., the period of time over which a multimeric polypeptide of the present disclosure, an RVV of the present disclosure is administered, can vary, depending on any of a variety of factors, e.g., patient response, etc. For example, an RVV of the present disclosure can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

An RVV of the present disclosure is administered to an individual using any available method and route suitable for drug delivery, including in vivo and ex vivo methods, as well as systemic and localized routes of administration.

Conventional and pharmaceutically acceptable routes of administration include intratumoral, peritumoral, intramuscular, intratracheal, intrathecal, intracranial, subcutaneous, intradermal, topical application, intravenous, intraarterial, intraperitoneal, intrabladder, rectal, nasal, oral, and other enteral and parenteral routes of administration. Routes of administration may be combined, if desired, or adjusted depending upon the RVV and/or the desired effect. An RVV of the present disclosure can be administered in a single dose or in multiple doses.

In some cases, an RVV of the present disclosure is administered intravenously. In some cases, an RVV of the present disclosure is administered intramuscularly. In some cases, an RVV of the present disclosure is administered locally. In some cases, an RVV of the present disclosure is administered intratumorally. In some cases, an RVV of the present disclosure is administered peritumorally. In some cases, an RVV of the present disclosure is administered intracranially. In some cases, an RVV of the present disclosure is administered subcutaneously. In some cases, an RVV of the present disclosure is administered intra-arterially. In some cases, an RVV of the present disclosure is administered intraperitoneally. In some cases, an RVV of the present disclosure is administered via an intrabladder route of administration. In some cases, an RVV of the present disclosure is administered intrathecally.

### D-2. Combinations

In some cases, an RVV of the present disclosure is administered in combination with another therapy or agent. For example, the RVV may be administered as an adjuvant therapy to a standard cancer therapy, administered in combination with another cancer therapy, or administered in combination with an agent that enhances the anti-tumor effect of the RVV. Standard cancer therapies include surgery (e.g., surgical removal of cancerous tissue), radiation therapy, bone marrow transplantation, chemotherapeutic treatment, antibody treatment, biological response modifier treatment, immunotherapy treatment, and certain combinations of the foregoing. In some cases, a method or use of the present disclosure comprises: a) administering to an individual in need thereof an RVV of the present disclosure, or a composition comprising same; and b) administering to the individual a second cancer therapy. In some cases, the second cancer therapy is selected from chemotherapy, biological therapy, radiotherapy, immunotherapy, hormone therapy, anti-vascular therapy, cryotherapy, toxin therapy, oncolytic virus therapy (e.g., an oncolytic virus other than an RVV of the present disclosure), cell therapy, and surgery.

Radiation therapy includes, but is not limited to, x-rays or gamma rays that are delivered from either an externally applied source such as a beam, or by implantation of small radioactive sources.

Suitable antibodies for use in cancer treatment include, but are not limited to, e.g., avelumab (tradename Bavencio), trastuzumab (tradename Herceptin) , bevacizumab (tradename Avastin), cetuximab (tradename Erbitux), panitumumab (tradename Vectibix), ipilimumab (tradename Yervoy), rituximab (tradename Rituxan), alemtuzumab (tradename Lemtrada), ofatumumab (tradename Arzerra), oregovomab (tradename OvaRex), lambrolizumab (MK-3475), pertuzumab (tradename Perjeta), ranibizumab (tradename Lucentis) etc., and conjugated antibodies, e.g., gemtuzumab ozogamicin (tradename Mylortarg), Brentuximab vedotin (tradename Adcetris), ⁹⁰Y-labelled ibritumomab tiuxetan (tradename Zevalin), ¹³¹I-labelled tositumoma (tradename Bexxar), etc. Suitable antibodies for use in cancer treatment include, but are not limited to, e.g., ipilimumab targeting CTLA-4 (as used in the treatment of melanoma, prostate cancer, RCC); tremelimumab targeting CTLA-4 (as used in the treatment of CRC, gastric, melanoma, NSCLC); nivolumab targeting PD-1 (as used in the treatment of melanoma, NSCLC, RCC); MK-3475 targeting PD-1 (as used in the treatment of melanoma); pidilizumab targeting PD-1 (as used in the treatment of hematologic malignancies); BMS-936559 targeting PD-L1 (as used in the treatment of melanoma, NSCLC, Ovarian, RCC); MEDI4736 targeting PD-L1; MPDL33280A targeting PD-L1 (as used in the treatment of Melanoma); Rituximab targeting CD20 (as used in the treatment of Non-Hodgkin’s lymphoma); Ibritumomab tiuxetan and tositumomab (as used in the treatment of Lymphoma); brentuximab vedotin targeting CD30 (as used in the treatment of Hodgkin’s lymphoma); gemtuzumab ozogamicin targeting CD33 (as used in the treatment of acute myelogenous leukaemia); alemtuzumab targeting CD52 (as used in the treatment of chronic lymphocytic leukaemia); IGN101 and adecatumumab targeting EpCAM (as used in the treatment of epithelial tumors (breast, colon and lung)); labetuzumab targeting CEA (as used in the treatment of breast, colon, and lung tumors); huA33 targeting gpA33 (as used in the treatment of colorectal carcinoma); pemtumomab and oregovomab targeting mucins (as used in the treatment of breast, colon, lung, and ovarian tumors); CC49 (minretumomab) targeting TAG-72 (as used in the treatment of breast, colon, and lung tumors); cG250 targeting CAIX (as used in the treatment of renal cell carcinoma); J591 targeting PSMA (as used in the treatment of prostate carcinoma); MOv18 and MORAb-003 (farletuzumab) targeting folate-binding protein (as used in the treatment of ovarian tumors); 3F8, ch14.18 and KW-2871 targeting angliosides (such as GD2, GD3 and GM2) (as used in the treatment of Neuroectodermal tumors and some epithelial tumors); hu3S193 and IgN311 targeting Le y (as used in the treatment of breast, colon, lung and prostate tumors); bevacizumab targeting VEGF (as used in the treatment of tumor vasculature); IM-2C6 and CDP791 targeting VEGFR (as used in the treatment of epithelium-derived solid tumors); Etaracizumab targeting Integrin _V_3 (as used in the treatment of tumor vasculature); volociximab targeting Integrin _5_1 (as used in the treatment of tumor vasculature); cetuximab, panitumumab, nimotuzumab and 806 targeting EGFR (as used in the treatment of glioma, lung, breast, colon, and head and neck tumors); trastuzumab and pertuzumab targeting ERBB2 (as used in the treatment of breast, colon, lung, ovarian and prostate tumors); MM-121 targeting ERBB3 (as used in the treatment of breast, colon, lung, ovarian and prostate, tumors); AMG 102, METMAB and SCH 900105 targeting MET (as used in the treatment of breast, ovary and lung tumors); AVE1642, IMC-A12, MK-0646, R1507 and CP 751871 targeting IGF1R (as used in the treatment of glioma, lung, breast, head and neck, prostate and thyroid cancer); KB004 and IIIA4 targeting EPHA3 (as used in the treatment of Lung, kidney and colon tumors, melanoma, glioma and haematological malignancies); mapatumumab (HGS-ETR1) targeting TRAILR1 (as used in the treatment of colon, lung and pancreas tumors and hematological malignancies); HGS-ETR2 and CS-1008 targeting TRAILR2; denosumab targeting RANKL (as used in the treatment of prostate cancer and bone metastases); sibrotuzumab and F19 targeting FAP (as used in the treatment of colon, breast, lung, pancreas, and head and neck tumors); 81C6 targeting Tenascin (as used in the treatment of glioma, breast and prostate tumors); blinatumomab (tradename Blincyto) targeting CD3 (as used in the treatment of ALL); pembrolizumab targeting PD-1 as used in cancer immunotherapy; 9E10 antibody targeting c-Myc; and the like.

In some cases, a method or use of the present disclosure comprises administering: a) an effective amount of an RVV of the present disclosure; and b) an anti-PD-1 antibody. In some cases, a method or use of the present disclosure comprises administering: a) an effective amount of an RVV of the present disclosure; and b) an anti-PD-L1 antibody. Suitable anti-PD-1 antibodies include, but are not limited to, pembrolizumab (Keytruda®; MK-3475), nivolumab, pidilizumab (CT-011), AMP-224, AMP-514 (MEDI-0680), PDR001, and PF-06801591. Suitable anti-PD-L1 antibodies include, but are not limited to, BMS-936559 (MDX1105), durvalumab (MEDI4736; Imfinzi), and atezolizumab (MPDL33280A; Tecentriq). See, e.g., Sunshine and Taube (2015) Curr. Opin. Pharmacol. 23:32; and Heery et al. (2017) The Lancet Oncology 18:587; Iwai et al. (2017) J. Biomed. Sci. 24:26; Hu-Lieskovan et al. (2017) Annals of Oncology 28: issue Suppl. 5, mdx376.048; and U.S. Pat. Publication No. 2016/0159905.

In some cases, a suitable antibody is a bispecific antibody, e.g., a bispecific monoclonal antibody. Catumaxomab, blinatumomab, solitomab, pasotuxizumab, and flotetuzumab are non-limiting examples of bispecific antibodies suitable for use in cancer therapy. See, e.g., Chames and Baty (2009) MAbs 1:539; and Sedykh et al. (2018) Drug Des. Devel. Ther. 12:195.

Biological response modifiers suitable for use in connection with the methods of the present disclosure include, but are not limited to, (1) inhibitors of tyrosine kinase (RTK) activity; (2) inhibitors of serine/threonine kinase activity; (3) tumor-associated antigen antagonists, such as antibodies that bind specifically to a tumor antigen; (4) apoptosis receptor agonists; (5) interleukin-2; (6) interferon-α.; (7) interferon-y; (8) colony-stimulating factors; (9) inhibitors of angiogenesis; and (10) antagonists of tumor necrosis factor.

Chemotherapeutic agents are non-peptidic (i.e., non-proteinaceous) compounds that reduce proliferation of cancer cells, and encompass cytotoxic agents and cytostatic agents. Non-limiting examples of chemotherapeutic agents include alkylating agents, nitrosoureas, antimetabolites, antitumor antibiotics, plant (vinca) alkaloids, and steroid hormones.

Agents that act to reduce cellular proliferation are known in the art and widely used. Such agents include alkylating agents, such as nitrogen mustards, nitrosoureas, ethylenimine derivatives, alkyl sulfonates, and triazenes, including, but not limited to, mechlorethamine, cyclophosphamide (Cytoxan™), melphalan (L-sarcolysin), carmustine (BCNU), lomustine (CCNU), semustine (methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine (CYTOSAR-U), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatine, and gemcitabine.

Suitable natural products and their derivatives, (e.g., vinca alkaloids, antitumor antibiotics, enzymes, lymphokines, and epipodophyllotoxins), include, but are not limited to, Ara-C, paclitaxel (Taxol®), docetaxel (Taxotere®), deoxycoformycin, mitomycin-C, L-asparaginase, azathioprine; brequinar; alkaloids, e.g. vincristine, vinblastine, vinorelbine, vindesine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithramycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, etc.; and the like.

Other anti-proliferative cytotoxic agents are navelbene, CPT-11, anastrazole, letrazole, capecitabine, reloxafine, cyclophosphamide, ifosamide, and droloxafine.

Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), paclitaxel (Taxol®), Taxol® derivatives, docetaxel (Taxotere®), thiocolchicine (NSC 361792), trityl cysterin, vinblastine sulfate, vincristine sulfate, natural and synthetic epothilones including but not limited to, eopthilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, etc.; estrogens and pregestins, e.g. hydroxyprogesterone caproate, medroxyprogesterone acetate, megestrol acetate, estradiol, clomiphene, tamoxifen; etc.; and adrenocortical suppressants, e.g. aminoglutethimide; 17α-ethinylestradiol; diethylstilbestrol, testosterone, fluoxymesterone, dromostanolone propionate, testolactone, methylprednisolone, methyltestosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and Zoladex®. Estrogens stimulate proliferation and differentiation, therefore compounds that bind to the estrogen receptor are used to block this activity. Corticosteroids may inhibit T cell proliferation.

Other chemotherapeutic agents include metal complexes, e.g. cisplatin (cis-DDP), carboplatin, etc.; ureas, e.g. hydroxyurea; and hydrazines, e.g. N-methylhydrazine; epidophyllotoxin; a topoisomerase inhibitor; procarbazine; mitoxantrone; leucovorin; tegafur; etc. Other anti-proliferative agents of interest include immunosuppressants, e.g. mycophenolic acid, thalidomide, desoxyspergualin, azasporine, leflunomide, mizoribine, azaspirane (SKF 105685); 4-(3-chloro-4-fluorophenylamino)-7-methoxy-6-(3-(4-morpholinyl)propoxy)quinazoline) (tradename Iressa); etc.

“Taxanes” include paclitaxel, as well as any active taxane derivative or pro-drug. “Paclitaxel” as used herein refer to not only the common chemically available form of paclitaxel, but analogues, formulations, and derivatives such as, for example, docetaxel, TAXOL®, TAXOTERE® (a formulation of docetaxel), 10-desacetyl analogs of paclitaxel, and 3′N-desbenzoyl-3′N-t-butoxycarbonyl analogs of paclitaxel, as well as paclitaxel conjugates (e.g., paclitaxel-PEG, paclitaxel-dextran, or paclitaxel-xylose).

Cell therapy includes chimeric antigen receptor (CAR) T cell therapy (CAR-T therapy); natural killer (NK) cell therapy; dendritic cell (DC) therapy (e.g., DC-based vaccine); T cell receptor (TCR) engineered T cell-based therapy; and the like.

### D-3. Cancers

Cancer cells that may be treated by methods, uses and compositions of the present disclosure include cancer cells from or in the bladder, blood, bone, bone marrow, brain, breast, colon, esophagus, gastrointestine, gum, head, kidney, liver, lung, nasopharynx, neck, ovary, prostate, skin, stomach, spinal cord, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget’s disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi’s sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing’s sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin’s disease; Hodgkin’s; paragranuloma; malignant lymphoma, small lymphocytic; malignant lymphoma, large cell, diffuse; malignant lymphoma, follicular; mycosis fungoides; other specified non-Hodgkin’s lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; pancreatic cancer; rectal cancer; and hairy cell leukemia.

Tumors that can be treated using a method or use of the present disclosure include, e.g., a brain cancer tumor, a head and neck cancer tumor, an esophageal cancer tumor, a skin cancer tumor, a lung cancer tumor, a thymic cancer tumor, a stomach cancer tumor, a colon cancer tumor, a liver cancer tumor, an ovarian cancer tumor, a uterine cancer tumor, a bladder cancer tumor, a testicular cancer tumor, a rectal cancer tumor, a breast cancer tumor, or a pancreatic cancer tumor.

In some cases, the tumor is a colorectal adenocarcinoma. In some cases, the tumor is non-small cell lung carcinoma. In some cases, the tumor is a triple-negative breast cancer. In some cases, the tumor is a solid tumor. In some cases, the tumor is a liquid tumor. In some cases, the tumor is recurrent. In some cases, the tumor is a primary tumor. In some cases, the tumor is metastatic.

### D-4. Subjects Suitable for Treatment

A variety of subjects are suitable for treatment with a subject method of treating cancer. Suitable subjects include any individual, e.g., a human or non-human animal who has cancer, who has been diagnosed with cancer, who is at risk for developing cancer, who has had cancer and is at risk for recurrence of the cancer, who has been treated with an agent other than a an oncolytic vaccinia virus of the present disclosure for the cancer and failed to respond to such treatment, or who has been treated with an agent other than an oncolytic vaccinia virus of the present disclosure for the cancer but relapsed after initial response to such treatment. In some embodiments, the subject treated is a human.

### D-5. Vaccinia Virus Immunogenic Compositions

In another aspect, the RVV provided by the present disclosure further comprises, in its genome, a nucleotide sequence encoding a cancer antigen (also referred to herein as a “cancer-associated antigen”). Thus, the present disclosure provides an RVV comprising, in its genome: i) a nucleotide sequence encoding an IL-2v polypeptide, where the IL-2v polypeptide has reduced binding to CD25 or otherwise having reduced undesirable properties, compared to wild-type IL-2; ii) an nucleotide sequence encoding a heterologous TK polypeptide; and iii) a nucleotide sequence encoding a cancer antigen. Such an RVV, when administered to an individual in need thereof (e.g., an individual having a cancer), can induce or enhance an immune response in the individual to the encoded cancer antigen. The immune response can reduce the number of cancer cells in the individual. In some cases, the RVV is replication competent. In some cases, the RVV is replication incompetent. In some cases, the RVV is not oncolytic. Suitable IL-2v polypeptides are as described above.

Examples of cancer-associated antigens include: α-folate receptor; carbonic anhydrase IX (CAIX); CD19; CD20; CD22; CD30; CD33; CD44v7/8; carcinoembryonic antigen (CEA); epithelial glycoprotein-2 (EGP-2); epithelial glycoprotein-40 (EGP-40); folate binding protein (FBP); fetal acetylcholine receptor; ganglioside antigen GD2; Her2/neu; IL-13R-a2; kappa light chain; LeY; L1 cell adhesion molecule; melanoma-associated antigen (MAGE); MAGE-A1; mesothelin; MUC1; NKG2D ligands; oncofetal antigen (h5T4); prostate stem cell antigen (PSCA); prostate-specific membrane antigen (PSMA); tumor-associate glycoprotein-72 (TAG-72); vascular endothelial growth factor receptor-2 (VEGF-R2) (See, e.g., Vigneron et al. (2013) Cancer Immunity 13:15; and Vigneron (2015) BioMed Res. Int’l Article ID 948501; and epidermal growth factor receptor (EGFR) vIII polypeptide (see, e.g., Wong et al. (1992) Proc. Natl. Acad. Sci. USA 89:2965; and Miao et al. (2014) PLoSOne 9:e94281); a MUC1 polypeptide; a human papillomavirus (HPV) E6 polypeptide; an LMP2 polypeptide; an HPV E7 polypeptide; an epidermal growth factor receptor (EGFR) vIII polypeptide; a HER-2/neu polypeptide; a melanoma antigen family A, 3 (MAGE A3) polypeptide; a p53 polypeptide; a mutant p53 polypeptide; an NY-ESO-1 polypeptide; a folate hydrolase (prostate-specific membrane antigen; PSMA) polypeptide; a carcinoembryonic antigen (CEA) polypeptide; a melanoma antigen recognized by T-cells (melanA/MART1) polypeptide; a Ras polypeptide; a gp100 polypeptide; a proteinase3 (PR1) polypeptide; a bcr-abl polypeptide; a tyrosinase polypeptide; a survivin polypeptide; a prostate specific antigen (PSA) polypeptide; an hTERT polypeptide; a sarcoma translocation breakpoints polypeptide; a synovial sarcoma X (SSX) breakpoint polypeptide; an EphA2 polypeptide; a prostate acid phosphatase (PAP) polypeptide; a melanoma inhibitor of apoptosis (ML-IAP) polypeptide; an alpha-fetoprotein (AFP) polypeptide; an epithelial cell adhesion molecule (EpCAM) polypeptide; an ERG (TMPRSS2 ETS fusion) polypeptide; a NA17 polypeptide, a paired-box-3 (PAX3) polypeptide; an anaplastic lymphoma kinase (ALK) polypeptide; an androgen receptor polypeptide; a cyclin B1 polypeptide; an N-myc proto-oncogene (MYCN) polypeptide; a Ras homolog gene family member C (RhoC) polypeptide; a tyrosinase-related protein-2 (TRP-2) polypeptide; a mesothelin polypeptide; a prostate stem cell antigen (PSCA) polypeptide; a melanoma associated antigen-1 (MAGE A1) polypeptide; a cytochrome P450 1B1 (CYP1B1) polypeptide; a placenta-specific protein 1 (PLAC1) polypeptide; a BORIS polypeptide (also known as CCCTC-binding factor or CTCF); an ETV6-AML polypeptide; a breast cancer antigen NY-BR-1 polypeptide (also referred to as ankyrin repeat domain-containing protein 30A); a regulator of G-protein signaling (RGS5) polypeptide; a squamous cell carcinoma antigen recognized by T-cells (SART3) polypeptide; a carbonic anhydrase IX polypeptide; a paired box-5 (PAX5) polypeptide; an OY-TES1 (testis antigen; also known as acrosin binding protein) polypeptide; a sperm protein 17 polypeptide; a lymphocyte cell-specific protein-tyrosine kinase (LCK) polypeptide; a high molecular weight melanoma associated antigen (HMW-MAA); an A-kinase anchoring protein-4 (AKAP-4); a synovial sarcoma X breakpoint 2 (SSX2) polypeptide; an X antigen family member 1 (XAGE1) polypeptide; a B7 homolog 3 (B7H3; also known as CD276) polypeptide; a legumain polypeptide (LGMN1; also known as asparaginyl endopeptidase); a tyrosine kinase with Ig and EGF homology domains-2 (Tie-2; also known as angiopoietin-1 receptor) polypeptide; a P antigen family member 4 (PAGE4) polypeptide; a vascular endothelial growth factor receptor 2 (VEGF2) polypeptide; a MAD-CT-1 polypeptide; a fibroblast activation protein (FAP) polypeptide; a platelet derived growth factor receptor beta (PDGFβ) polypeptide; a MAD-CT-2 polypeptide; a Fos-related antigen-1 (FOSL) polypeptide; and a Wilms tumor-1 (WT-1) polypeptide.

Amino acid sequences of cancer-associated antigens are known in the art; see, e.g., MUC1 (GenBank CAA56734); LMP2 (GenBank CAA47024); HPV E6 (GenBank AAD33252); HPV E7 (GenBank AHG99480); EGFRvIII (GenBank NP_001333870); HER-2/neu (GenBank AAI67147); MAGE-A3 (GenBank AAH11744); p53 (GenBank BAC16799); NY-ESO-1 (GenBank CAA05908); PSMA (GenBank AAH25672); CEA (GenBank AAA51967); melan/MART1 (GenBank NP_005502); Ras (GenBank NP_001123914); gp100 (GenBank AAC60634); bcr-abl (GenBank AAB60388); tyrosinase (GenBank AAB60319); survivin (GenBank AAC51660); PSA (GenBank CAD54617); hTERT (GenBank BAC11010); SSX (GenBank NP_001265620); Eph2A (GenBank NP_004422); PAP (GenBank AAH16344); ML-IAP (GenBank AAH14475); AFP (GenBank NP_001125); EpCAM (GenBank NP_002345); ERG (TMPRSS2 ETS fusion) (GenBank ACA81385); PAX3 (GenBank AAI01301); ALK (GenBank NP_004295); androgen receptor (GenBank NP_000035); cyclin B1 (GenBank CAO99273); MYCN (GenBank NP_001280157); RhoC (GenBank AAH52808); TRP-2 (GenBank AAC60627); mesothelin (GenBank AAH09272); PSCA (GenBank AAH65183); MAGE A1 (GenBank NP_004979); CYP1B1 (GenBank AAM50512); PLAC1 (GenBank AAG22596); BORIS (GenBank NP_001255969); ETV6 (GenBank NP_001978); NY-BR1 (GenBank NP_443723); SART3 (GenBank NP_055521); carbonic anhydrase IX (GenBank EAW58359); PAX5 (GenBank NP_057953); OY-TES1 (GenBank NP_115878); sperm protein 17 (GenBank AAK20878); LCK (GenBank NP_001036236); HMW-MAA (GenBank NP_001888); AKAP-4 (GenBank NP_003877); SSX2 (GenBank CAA60111); XAGE1 (GenBank NP_001091073; XP_001125834; XP_001125856; and XP_001125872); B7H3 (GenBank NP_001019907; XP_947368; XP_950958; XP_950960; XP_950962; XP_950963; XP_950965; and XP_950967); LGMN1 (GenBank NP_001008530); TIE-2 (GenBank NP_000450); PAGE4 (GenBank NP_001305806); VEGFR2 (GenBank NP_002244); MAD-CT-1 (GenBank NP_005893 NP_056215); FAP (GenBank NP_004451); PDGFβ (GenBank NP_002600); MAD-CT-2 (GenBank NP_001138574); FOSL (GenBank NP_005429); and WT-1 (GenBank NP_000369). These polypeptides are also discussed in, e.g., Cheever et al. (2009) Clin. Cancer Res. 15:5323, and references cited therein; Wagner et al. (2003) J. Cell. Sci. 116:1653; Matsui et al. (1990) Oncogene 5:249; and Zhang et al. (1996) Nature 383:168.

As noted above, in some cases, an RVV of the present disclosure is replication incompetent. In some cases, the RVV comprises a modification of a vaccinia virus gene that results in inability of the vaccinia virus to replicate. One or more vaccinia virus genes encoding gene products required for replication can be modified such that the vaccinia virus is unable to replicate. For example, an RVV can be modified to reduce the levels and/or activity of an intermediate transcription factor (e.g., A8R and/or A23R) (see, e.g., Wyatt et al. (2017) mBio 8:e00790; and Warren et al. (2012) J. Virol. 86:9514) and/or a late transcription factor (e.g., one or more of G8R, A1L, and A2L) (see, e.g., Yang et al. (2013) Virology 447:213). Reducing the levels and/or activity of an intermediate transcription factor and/or a late transcription factor can result in a modified vaccinia virus that can express polypeptide(s) encoded by a nucleotide sequence(s) that is operably linked to an early viral promoter; however, the virus will be unable to replicate. Modifications include, e.g., deletion of all or part of the gene; insertion into the gene; and the like. For example, all or a portion of the A8R gene can be deleted. As another example, all or a portion of the A23R gene can be deleted. As another example, all or a portion of the G8R gene can be deleted. As another example, all or a portion of the A1L gene can be deleted. As another example, all or a portion of the A2L gene can be deleted.

### D-5. Administration of Analogs of 2′-deoxyguanosine

In another aspect, the present disclosure provides administration of the RVV described herein in combination with a synthetic analog of 2′-deoxyguanosine.

Oncolytic viruses may cause adverse side effects in a subject who received administration of the virus. Examples of the side effects include skin lesions, such vesicular lesions or “vesicular rash.” In some embodiments, the present disclosure provides a method of treating cancer in an individual, comprising administering to the individual: b) an effective amount of a replication-competent, RVV of the present disclosure; and b) an effective amount of a synthetic analog of 2′-deoxy-guanosine. In some other embodiments, the present disclosure provides a method of treating, reducing, or managing a side effect of the oncolytic RVV of the present disclosure, which comprises administering an effective amount of a synthetic analog of 2′-deoxy-guanosine to a subject who has received administration of the oncolytic RVV.

An “effective amount” of a synthetic analog of 2′-deoxy-guanosine is an amount that is effective to reduce an adverse side effect of administration of a replication-competent, RVV of the present disclosure. For example, where the adverse side effect is skin lesions, an effective amount of a synthetic analog of 2′-deoxy-guanosine is an amount that, when administered to an individual in one or more doses, is effective to reduce the number and/or severity and/or duration of vaccinia virus-induced skin lesions in the individual. For example, an effective amount of a synthetic analog of 2′-deoxy-guanosine can be an amount that, when administered to an individual in one or more doses, is effective to reduce the number and/or severity and/or duration of vaccinia virus-induced skin lesions in the individual by at least 10%, at least 15%, at least 20%, at least 25%, at least 30%, at least 40%, at least 50%, at least 60%, at least 75%, or more than 75%, compared with the number and/or severity and/or duration of vaccinia virus-induced skin lesions in the individual prior to administration of the synthetic analog of 2′-deoxy-guanosine or in the absence of administration of the synthetic analog of 2′-deoxy-guanosine. In some cases, an effective amount of a synthetic analog of 2′-deoxy-guanosine is an amount that, when administered to an individual in one or more doses, is effective to reduce shedding of virus from vaccinia virus-induced skin lesions. For example, in some cases, an effective amount of a synthetic analog of 2′-deoxy-guanosine is an amount that, when administered to an individual in one or more doses, is effective to reduce shedding of virus from vaccinia virus-induced skin lesions by at least 10%, at least 15%, at least 20%, at least 25%, at least 30%, at least 40%, at least 50%, at least 60%, at least 75%, or more than 75%, compared with the level or degree of virus shedding from vaccinia virus-induced skin lesions in the individual prior to administration of the synthetic analog of 2′-deoxy-guanosine or in the absence of administration of the synthetic analog of 2′-deoxy-guanosine. Where the adverse side effect is a skin lesion, in some cases, the synthetic analog of 2′-deoxy-guanosine can be administered by any convenient route of administration (e.g., topically, orally, intravenously, etc.). For example, where the adverse side effect is a skin lesion, in some cases, the synthetic analog of 2′-deoxy-guanosine can be administered topically. For reducing skin lesions, the synthetic analog of 2′-deoxy-guanosine is typically administered topically, for example, by application of the 2′-deoxy-guanosine analog to the lesion area of the skin.

Administration of a synthetic analog of 2′-deoxy-guanosine reduces replication of a replication-competent RVV of the present disclosure. Such reduction in replication of the replication-competent RVV of the present disclosure may be desirable, e.g., to control the level of replication-competent RVV in an individual, to control the effect of the replication-competent RVV, and the like. Thus, in some embodiments, the preset disclosure provides a method of controlling the replication of the replication-competent RVV provided by the present disclosure in an individual who is administered the RVV, comprising administering to the individual an effective amount of an anti-viral drug, such as a 2′-deoxy-guanosine analog. In some specific embodiments, the 2′-deoxy-guanosine analog administered is effective to reduce replication of a replication-competent RVV of the present disclosure in an individual by at least 10%, at least 15%, at least 20%, at least 25%, at least 30%, at least 40%, at least 50%, at least 60%, at least 75%, or more than 75%, compared with the level of replication of the replication-competent, RVV in the individual prior to administration of the 2′-deoxy-guanosine analog or in the absence of administration of the 2′-deoxy-guanosine analog.

In some other embodiments, the preset disclosure provides a method of treating cancer in an individual, comprising: a) administering an effective amount of a replication-competent RVV of the present disclosure; and b) administering an effective amount of a synthetic analog of 2′-deoxy-guanosine. In some cases, an effective amount of a synthetic analog of 2′-deoxy-guanosine is an amount that, when administered to an individual in one or more doses, is effective to reduce replication of a replication-competent RVV of the present disclosure in an individual by at least 10%, at least 15%, at least 20%, at least 25%, at least 30%, at least 40%, at least 50%, at least 60%, at least 75%, or more than 75%, compared with the level of replication of the replication-competent, RVV in the individual prior to administration of the synthetic analog of 2′-deoxy-guanosine or in the absence of administration of the synthetic analog of 2′-deoxy-guanosine.

A synthetic analog of 2′-deoxy-guanosine can be administered after administration of a replication-competent RVV of the present disclosure. For example, a synthetic analog of 2′-deoxy-guanosine can be administered 1 day to 7 days, from 7 days to 2 weeks, from 2 weeks to 1 month, from 1 month to 3 months, or more than 3 months, after administration of the replication-competent, recombinant oncolytic vaccinia virus.

In some cases, administration of a synthetic analog of 2′-deoxy-guanosine to an individual to whom a replication-competent RVV of the present disclosure has been administered, induces rapid, systemic, tumor lysis (lysis of cancer cells) in the individual. For example, a synthetic analog of 2′-deoxy-guanosine can be administered to an individual once oncolytic vaccinia virus-induced slowing of tumor growth has occurred and/or once viral replication is at or just after its peak and/or once circulating antibody to vaccinia virus proteins are at or just after their peak. Whether slowing of tumor growth has occurred, following administration of a replication-competent RVV of the present disclosure, can be determined using any of a variety of established methods to measure tumor growth and/or cancer cell number. Whether replication of a replication-competent RVV of the present disclosure in an individual is at its peak or just after its peak can be determined by detecting and/or measuring levels of TKv polypeptide in the individual, as described herein, where a non-limiting example of a suitable method is PET. Whether circulating antibody to a replication-competent RVV of the present disclosure is at or just after its peak can be measured using standard methods for measuring the levels of an antibody, where such methods include, e.g., enzyme-linked immunosorbent assay (ELISA), radioimmunoassay (RIA), and the like.

As an example, a method of use of the present disclosure can comprise: a) administering to an individual in need thereof an effective amount of a replication-competent RVV of the present disclosure; b) measuring: i) tumor size and/or cancer cell number in the individual; and/or ii) levels of TKv polypeptide in the individual; and/or iii) levels of antibody to the replication-competent, in the individual; and c) where the measuring step indicates that: i) tumor growth has slowed and/or the number of cancer cells has decreased, compared to the tumor growth and/or the number of cancer cells before administration of the replication-competent, recombinant oncolytic vaccinia virus; and/or ii) the level of TKv polypeptide in the individual is at or just past its peak; and/or iii) the level of circulating antibody to the replication-competent RVV in the individual is at or just past its peak, administering a synthetic analog of 2′-deoxy-guanosine. For example, a method or use of the present disclosure can comprise: a) administering to an individual in need thereof an effective amount of a replication-competent RVV of the present disclosure; and b) administering to the individual an effective amount of a synthetic analog of 2′-deoxy-guanosine, where the administration step (b) is carried out from 5 days to 20 days (e.g., 5 days, 6 days, 7 days, 8 days, 9 days, 10 days, 11 days, 12 days, 13 days, 14 days, 15 days, 16 days, 17 days, 18 days, 19 days, or 20 days) after step (a).

Suitable synthetic analogs of 2′-deoxy-guanosine include, e.g., acyclovir (acycloguanosine), 5′-iododeoxyuridine (also referred to as “idoxuridine”), ganciclovir, valganciclovir, famciclovir, valaciclovir, 2′-fluoro-2′-deoxy-5-iodo-1-beta-d-arabinofuranosyluracil (FIAU), and the like. The structures of suitable synthetic analogs of 2′-deoxy-guanosine are shown below. ganciclovir:

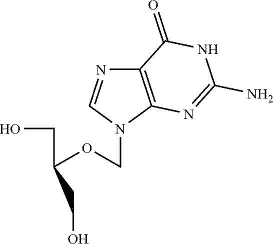

valganciclovir:

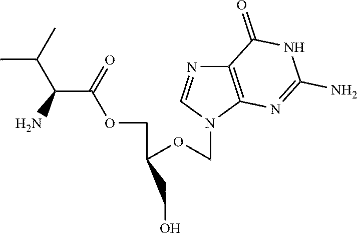 [SMILES: CC(C)[C@H](N)C(=O)OC[C@H](CO)OCn1cnc2c(=O)[nH]c(N)nc21]

valaciclovir:

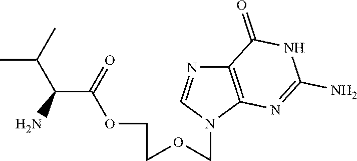 [SMILES: CC(C)[C@H](N)C(=O)OCCOCn1cnc2c(=O)[nH]c(N)nc21]

famciclovir:

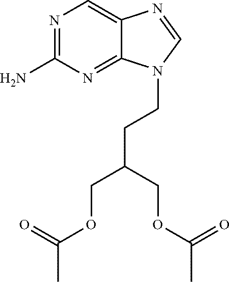 [SMILES: CC(=O)OCC(CCn1cnc2cnc(N)nc21)COC(C)=O]

In some cases, a synthetic analog of 2′-deoxy-guanosine is administered in a dose of less than 4000 mg per day orally. In some cases, a suitable oral dose of a synthetic analog of 2′-deoxy-guanosine is in the range of from about 50 mg per day to about 2500 mg per day, e.g., from about 50 mg per day to about 100 mg per day, from about 100 mg per day to about 200 mg per day, from about 200 mg per day to about 300 mg per day, from about 300 mg per day to about 400 mg per day, from about 400 mg per day to about 500 mg per day, from about 500 mg per day to about 600 mg per day, from about 600 mg per day to about 700 mg per day, from about 700 mg per day to about 800 mg per day, from about 800 mg per day to about 900 mg per day, from about 900 mg per day to about 1000 mg per day, from about 1000 mg per day to about 1250 mg per day, from about 1250 mg per day to about 1500 mg per day, from about 1500 mg per day to about 1750 mg per day, from about 1750 mg per day to about 2000 mg per day, from about 2000 mg per day to about 2250 mg per day, or from about 2250 mg per day to about 2500 mg per day. In some cases, a suitable oral dose of a synthetic analog of 2′-deoxy-guanosine is in the range of from about 2500 mg per day to about 3000 mg per day, from about 3000 mg per day to about 3500 mg per day, or from about 3500 mg per day to about 4000 mg per day.

As one non-limiting example, ganciclovir administered in a dose of 1000 mg 3 times per day, for a total daily dose of 3000 mg. Ganciclovir can be administered in a total daily dose of less than 3000 mg (e.g., from about 50 mg per day to about 2500 mg per day, e.g., from about 50 mg per day to about 100 mg per day, from about 100 mg per day to about 200 mg per day, from about 200 mg per day to about 300 mg per day, from about 300 mg per day to about 400 mg per day, from about 400 mg per day to about 500 mg per day, from about 500 mg per day to about 600 mg per day, from about 600 mg per day to about 700 mg per day, from about 700 mg per day to about 800 mg per day, from about 800 mg per day to about 900 mg per day, from about 900 mg per day to about 1000 mg per day, from about 1000 mg per day to about 1250 mg per day, from about 1250 mg per day to about 1500 mg per day, from about 1500 mg per day to about 1750 mg per day, from about 1750 mg per day to about 2000 mg per day, from about 2000 mg per day to about 2250 mg per day, or from about 2250 mg per day to about 2500 mg per day). In some cases, ganciclovir is administered via oral administration.

As another non-limiting example, acyclovir can be administered in a total daily dose of from 1000 mg to 4000 mg. Acyclovir can be administered in a total daily dose of less than 4000 mg (e.g., from about 50 mg per day to about 2500 mg per day, e.g., from about 50 mg per day to about 100 mg per day, from about 100 mg per day to about 200 mg per day, from about 200 mg per day to about 300 mg per day, from about 300 mg per day to about 400 mg per day, from about 400 mg per day to about 500 mg per day, from about 500 mg per day to about 600 mg per day, from about 600 mg per day to about 700 mg per day, from about 700 mg per day to about 800 mg per day, from about 800 mg per day to about 900 mg per day, from about 900 mg per day to about 1000 mg per day, from about 1000 mg per day to about 1250 mg per day, from about 1250 mg per day to about 1500 mg per day, from about 1500 mg per day to about 1750 mg per day, from about 1750 mg per day to about 2000 mg per day, from about 2000 mg per day to about 2250 mg per day, or from about 2250 mg per day to about 2500 mg per day). In some cases, acyclovir is administered via oral administration.

As another example valganciclovir is administered in a total daily dose of from about to about . Valganciclovir can be administered in a total daily dose of less than 1800 mg (e.g., from about 500 mg/day to about 600 mg/day, from about 600 mg/day to about 700 mg/day, from about 700 mg/day to about 800 mg/day, from about 800 mg/day to about 900 mg/day, from about 900 mg/day to about 1000 mg/day, from about 1000 mg/day to about 1200 mg/day, from about 1200 mg/day to about 1400 mg/day, or from about 1400 mg/day to about 1600 mg/day). In some cases, valganciclovir is administered via oral administration.

As another example, famciclovir is administered in a total daily dose of from about 2000 mg/day to about 4000 mg/day. Famciclovir can be administered in a total daily dose of less than 4000 mg (e.g., from about 50 mg per day to about 2500 mg per day, e.g., from about 50 mg per day to about 100 mg per day, from about 100 mg per day to about 200 mg per day, from about 200 mg per day to about 300 mg per day, from about 300 mg per day to about 400 mg per day, from about 400 mg per day to about 500 mg per day, from about 500 mg per day to about 600 mg per day, from about 600 mg per day to about 700 mg per day, from about 700 mg per day to about 800 mg per day, from about 800 mg per day to about 900 mg per day, from about 900 mg per day to about 1000 mg per day, from about 1000 mg per day to about 1250 mg per day, from about 1250 mg per day to about 1500 mg per day, from about 1500 mg per day to about 1750 mg per day, from about 1750 mg per day to about 2000 mg per day, from about 2000 mg per day to about 2250 mg per day, or from about 2250 mg per day to about 2500 mg per day). In some cases, famciclovir is administered via oral administration.

As another example valacyclovir is administered in a total daily dose of from about 2000 mg to about 4000 mg. Valacyclovir can be administered in a total daily dose of less than 4000 mg (e.g., from about 50 mg per day to about 2500 mg per day, e.g., from about 50 mg per day to about 100 mg per day, from about 100 mg per day to about 200 mg per day, from about 200 mg per day to about 300 mg per day, from about 300 mg per day to about 400 mg per day, from about 400 mg per day to about 500 mg per day, from about 500 mg per day to about 600 mg per day, from about 600 mg per day to about 700 mg per day, from about 700 mg per day to about 800 mg per day, from about 800 mg per day to about 900 mg per day, from about 900 mg per day to about 1000 mg per day, from about 1000 mg per day to about 1250 mg per day, from about 1250 mg per day to about 1500 mg per day, from about 1500 mg per day to about 1750 mg per day, from about 1750 mg per day to about 2000 mg per day, from about 2000 mg per day to about 2250 mg per day, or from about 2250 mg per day to about 2500 mg per day). In some cases, valacyclovir is administered via oral administration.

As another example, ganciclovir is administered in a total daily dose of about 10 mg/kg. Ganciclovir can be administered in a total daily dose of less than 10 mg/kg (e.g., from about 1 mg/kg to about 2 mg/kg, from about 2 mg/kg to about 3 mg/kg, from about 3 mg/kg to about 4 mg/kg, from about 4 mg/kg to about 5 mg/kg, from about 5 mg/kg to about 6 mg/kg, from about 6 mg/kg to about 7 mg/kg, from about 7 mg/kg to about 8 mg/kg, or from about 8 mg/kg to about 9 mg/kg). In some cases, ganciclovir is administered via injection (e.g., intramuscular injection, intravenous injection, or subcutaneous injection).

As another example, acyclovir is administered in a total daily dose of from about 15 mg/kg to about 30 mg/kg, or from about 30 mg/kg to about 45 mg/kg. Acyclovir can be administered in a total daily dose of less than 45 mg/kg (e.g., from about 5 mg/kg to about 7.5 mg/kg, from about 7.5 mg/kg to about 10 mg/kg, from about 10 mg/kg to about 12.5 mg/kg, from about 12.5 mg/kg to about 15 mg/kg, from about 15 mg/kg to about 20 mg/kg, from about 20 mg/kg to about 25 mg/kg, from about 25 mg/kg to about 30 mg/kg, or from about 30 mg/kg to about 35 mg/kg. In some cases, acyclovir is administered via injection (e.g., intramuscular injection, intravenous injection, or subcutaneous injection).

As another example, valganciclovir is administered in a total daily dose of about 10 mg/kg. Valganciclovir can be administered in a total daily dose of less than 10 mg/kg (e.g., from about 1 mg/kg to about 2 mg/kg, from about 2 mg/kg to about, from about 3 mg/kg to about 4 mg/kg, from about 4 mg/kg to about 5 mg/kg, from about 5 mg/kg to about 6 mg/kg, from about 6 mg/kg to about 7 mg/kg, from about 7 mg/kg to about 8 mg/kg, or from about 8 mg/kg to about 9 mg/kg). In some cases, valganciclovir is administered via injection (e.g., intramuscular injection, intravenous injection, or subcutaneous injection).

In some cases, a synthetic analog of 2′-deoxy-guanosine is administered topically. Formulations suitable for topical administration include, e.g., dermal formulations (e.g., liquids, creams, gels, and the like) and ophthalmic formulations (e.g., creams, liquids, gels, and the like). Topical doses of ganciclovir can be, e.g., 1 drop of a 0.15% formulation 5 times per day, e.g., for ophthalmic indications. Topical doses of acyclovir can be, e.g., application 6 times per day of a 5% formulation in an amount sufficient to cover a skin lesion. Topical doses of idoxuridine can be, e.g., application every 4 hours of 1 drop of a 0.5% ointment or a 0.1% cream.

In some cases, a synthetic analog of 2′-deoxy-guanosine is administered in a dose less than 10 mg/kg body weight intravenously. In some cases, a suitable intravenous dose of a synthetic analog of 2′-deoxy-guanosine is in the range of from about 1 mg/kg body weight to about 2.5 mg/kg body weight, from about 2.5 mg/kg body weight to about 5 mg/kg body weight, from about 5 mg/kg body weight to about 7.5 mg/kg body weight, or from about 7.5 mg/kg body weight to about 10 mg/kg body weight.

### E. Examples of Non-Limiting Aspects of the Disclosure

Aspects, including embodiments, of the subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
Aspect 1. A RVV comprising, in its genome: (1) a nucleotide sequence encoding a variant interleukin-2 (IL-2v) polypeptide, wherein the IL-2v polypeptide has reduced undesirable properties as compared to the wild-type IL-2; and (2) a nucleotide sequence encoding a heterologous thymidine kinase (TK) polypeptide .
Aspect 2. The RVV of aspect 1, wherein the vaccinia virus further comprises a modification that renders the vaccinia thymidine kinase deficient.
Aspect 3. The vaccinia virus of aspect 2, wherein the modification results in a lack of J2R expression and/or function.
Aspect 4. The vaccinia virus of any one of aspects 1-3, wherein the vaccinia virus is a Copenhagen strain vaccinia virus.
Aspect 5. The vaccinia virus of any one of aspects 1-3, wherein the vaccinia virus is a WR strain vaccinia virus.
Aspect 6. The vaccinia virus of any one of aspects 1-5, wherein the vaccinia virus comprises an A34R gene comprising a K151E substitution.
Aspect 7. The vaccinia virus of any one of aspects 1-6, wherein the IL-2v polypeptide comprises substitutions of one or more of F42, Y45, and L72, based on the amino acid numbering of the IL-2 amino acid sequence depicted in SEQ ID NO: 1.
Aspect 8. The vaccinia virus of any one of aspects 1-7, wherein IL-2v polypeptide comprises an F42L, F42A, F42G, F42S, F42T, F42Q, F42E, F42D, F42R, or F42K substitution, based on the amino acid numbering of the IL-2 amino acid sequence depicted in SEQ ID NO:1.
Aspect 9. The vaccinia virus of any one of aspects 1-8, wherein IL-2v polypeptide comprises a Y45A, Y45G, Y45S, Y45T, Y45Q, Y45E, Y45N, Y45D, Y45R, or Y45K substitution, based on the amino acid numbering of the IL-2 amino acid sequence depicted in SEQ ID NO:1.
Aspect 10. The vaccinia virus of any one of aspects 1-9, wherein IL-2v polypeptide comprises CD25 is an L72G, L72A, L72S, L72T, L72Q, L72E, L72N, L72R, or L72K substitution, based on the amino acid numbering of the IL-2 amino acid sequence depicted in SEQ ID NO:1.
Aspect 11. The vaccinia virus of any one of aspects 1-10, wherein the IL-2v polypeptide comprises F42A, Y45A, and L72G substitutions, based on the amino acid numbering of the IL-2 amino acid sequence depicted in SEQ ID NO: 1.
Aspect 12. The vaccinia virus of any one of aspects 1-11, wherein the IL-2v polypeptide-encoding nucleotide sequence is operably linked to a regulatable promoter.
Aspect 13. The vaccinia virus of aspect 12, wherein the regulatable promoter is regulated by tetracycline or a tetracycline analog or derivative.
Aspect 14. A composition comprising: a) the vaccinia virus of any one of aspects 1-13; and b) a pharmaceutically acceptable excipient.
Aspect 15. A method of inducing oncolysis in an individual having a tumor, the method comprising administering to the individual an effective amount of the vaccinia virus of any one of aspects 1-13, or the composition of aspect 14.
Aspect 16. The method of aspect 15, wherein said administering comprises administering a single dose of the virus or the composition.
Aspect 17. The method of aspect 16, wherein the single dose comprises at least 10⁶ plaque forming units (pfu) of the vaccinia virus.
Aspect 18. The method of aspect 16, wherein the single dose comprises from 10⁹ to 10¹² pfu of the vaccinia virus.
Aspect 19. The method of aspect 15, wherein said administering comprises administering multiple doses of the vaccinia virus or the composition.
Aspect 20. The method of aspect 19, wherein the vaccinia virus or the composition is administered every other day.
Aspect 21. The method of any one of aspects 15-20, wherein the vaccinia virus or the composition is administered once per week.
Aspect 22. The method of any one of aspects 15-20, wherein the vaccinia virus or the composition is administered every other week.
Aspect 23. The method of any one of aspects 15-21, wherein the tumor is a brain cancer tumor, a head and neck cancer tumor, an esophageal cancer tumor, a skin cancer tumor, a lung cancer tumor, a thymic cancer tumor, a stomach cancer tumor, a colon cancer tumor, a liver cancer tumor, an ovarian cancer tumor, a uterine cancer tumor, a bladder cancer tumor, a testicular cancer tumor, a rectal cancer tumor, a breast cancer tumor, or a pancreatic cancer tumor.
Aspect 24. The method of any one of aspects 15-22, wherein the tumor is a colorectal adenocarcinoma.
Aspect 25. The method of any one of aspects 15-22, wherein the tumor is non-small cell lung carcinoma.
Aspect 26. The method of any one of aspects 15-22, wherein the tumor is a triple-negative breast cancer.
Aspect 27. The method of any one of aspects 15-22, wherein the tumor is a solid tumor.
Aspect 28. The method of any one of aspects 15-22, wherein the tumor is a liquid tumor.
Aspect 29. The method of any one of aspects 15-28, wherein the tumor is recurrent.
Aspect 30. The method of any one of aspects 15-28, wherein the tumor is a primary tumor.
Aspect 31. The method of any one of aspects 15-28, wherein the tumor is metastatic.
Aspect 32. The method of any one of aspects 15-31, further comprising administering to the individual a second cancer therapy.
Aspect 33. The method of aspect 32, wherein the second cancer therapy is selected from chemotherapy, biological therapy, radiotherapy, immunotherapy, hormone therapy, anti-vascular therapy, cryotherapy, toxin therapy, oncolytic virus therapy, a cell therapy, and surgery.
Aspect 34. The method of aspect 32, wherein the second cancer therapy comprises an anti-PD1 antibody or an anti-PD-L1 antibody.
Aspect 35. The method of any one of aspects 15-34, wherein the individual is immunocompromised.
Aspect 36. The method of any one of aspects 15-35, wherein said administering of the vaccinia virus or the composition is intratumoral.
Aspect 37. The method of any one of aspects 15-35, wherein said administering of the vaccinia virus or the composition is peritumoral.
Aspect 38. The method of any one of aspects 15-35, wherein said administering of the vaccinia virus or the composition is intravenous.
Aspect 39. The method of any one of aspects 15-35, wherein said administering of the vaccinia virus or the composition is intra-arterial.
Aspect 40. The method of any one of aspects 15-35, wherein said administering of the vaccinia virus or the composition is intrabladder.
Aspect 41. The method of any one of aspects 15-35, wherein said administering of the vaccinia virus or the composition is intrathecal.
Aspect 42. An RVV comprising, in its genome, a nucleotide sequence encoding a variant interleukin-2 (IL-2v) polypeptide, wherein the IL-2v polypeptide comprises one or more amino acid substitutions that provides for reduced binding to CD25, compared to wild-type IL-2.
Aspect 43. An RVV comprising, in its genome, a nucleotide sequence encoding a variant interleukin-2 (IL-2v) polypeptide comprising SEQ ID NO: 9, wherein the vaccinia virus is a Copenhagen strain vaccinia virus, is vaccinia thymidine kinase deficient, and comprises an A34R gene comprising a K151E substitution.
Aspect 44. The vaccinia virus of aspect 43, further comprising a signal peptide.
Aspect 45. The vaccinia virus of aspect 44, wherein the signal peptide comprises SEQ ID NO:22.
Aspect 46. An RVV comprising, in its genome, a variant interleukin-2 (IL-2v) nucleotide sequence comprising SEQ ID NO: 10, wherein the vaccinia virus is a Copenhagen strain vaccinia virus, is vaccinia thymidine kinase deficient, and comprises an A34R gene comprising a K151E substitution.
Aspect 47. An RVV comprising, in its genome, a variant interleukin-2 (IL-2v) nucleotide sequence comprising SEQ ID NO: 12, wherein the vaccinia virus is a Copenhagen strain vaccinia virus, is vaccinia thymidine kinase deficient, and comprises an A34R gene comprising a K151E substitution.
Aspect 48. A composition comprising: (i) the vaccinia virus of any one of aspects 42-47 and (ii) a pharmaceutically acceptable carrier.
Aspect 49. An RVV comprising, in its genome, a nucleotide sequence encoding a variant interleukin-2 (IL-2v) polypeptide, wherein the IL-2v polypeptide provides reduced undesirable biological activity when compared to wild-type IL-2.
Aspect 50. The vaccinia virus of any one of aspects 1-13, or the composition of aspect 14, for use in a method of inducing oncolysis in an individual having a tumor.
Aspect 51. Use of the vaccinia virus of any one of aspects 1-13, or the composition of aspect 14, in the manufacture of a medicament for inducing oncolysis in an individual having a tumor.

### F. Sequence Index

| SEQ ID NO | Type | Description |
|---|---|---|
| 1 | Protein | Amino acid sequence of mature form of a wild-type human IL-2 (hIL-2) polypeptide |
| 2 | DNA | Nucleotide sequence encoding a mouse IL-2v polypeptide comprising F76A, Y79A, and L106G substitutions (codon optimized for expression in mouse) |
| 3 | Protein | Amino acid sequences of a mouse IL-2v polypeptide (comprising F76A, Y79A, and L106G substitutions |
| 4 | DNA | Nucleotide sequence of homologous recombination donor fragment encoding an IL-2v polypeptide (VV27/VV38 homologous recombination donor fragment) |
| 5 | DNA | Nucleotide sequence of homologous recombination donor fragment encoding an IL-2v polypeptide (VV39 homologous recombination donor fragment) |
| 6 | DNA | Nucleotide Sequence of Copenhagen J2R homologous recombination plasmid |
| 7 | DNA | Nucleotide sequence of Copenhagen J2R homologous recombination plasmid containing mouse IL-2 variant polypeptide |
| 8 | DNA | Nucleotide sequence of Western Reserve J2R homologous recombination plasmid containing mouse IL-2 variant |
| 9 | Protein | Amino acid sequence of a human IL-2v polypeptide comprising F42A, Y45A, and L72G substitutions |
| 10 | DNA | Nucleotide sequence encoding a human IL-2v polypeptide comprising F42A, Y45A, and L72G substitutions (human codon-optimized) |
| 11 | DNA | Nucleotide sequence encoding a human IL-2v polypeptide comprising F42A, Y45A, and L72G substitutions (vaccinia virus codon-optimized) |
| 12 | DNA | Nucleotide sequence encoding a human IL-2v polypeptide comprising F62A, Y65A, and L92G substitutions |
| 13 | DNA | Nucleotide sequence encoding a human IL-2v polypeptide comprising F62A, Y65A, and L92G substitutions |
| 14 | Protein | Amino acid sequence of a human IL-2v polypeptide comprising F62A, Y65A, and L92G substitutions |
| 15 | DNA | Nucleotide sequence of VV75 homologous recombination donor fragment containing hIL-2v (human codon optimized) |
| 16 | DNA | Nucleotide sequence of Copenhagen J2R homologous recombination plasmid containing hIL-2v (human codon optimized) |
| 17 | DNA | Nucleotide sequence of homologous recombination donor fragment containing hIL-2v (vaccinia virus codon optimized), |
| 18 | DNA | Nucleotide sequence of Copenhagen J2R homologous recombination plasmid containing hIL-2v (vaccinia virus codon optimized) |
| 19 | DNA | Nucleotide sequence encoding a mouse IL-2v polypeptide (codon optimized for vaccinia virus) |
| 20 | DNA | Nucleotide sequence of a mouse IL-2v homologous recombination donor fragment (vaccinia virus codon optimized) |
| 21 | Protein | Amino acid sequence of the precursor form of a wild-type human IL-2 polypeptide (including signal peptide) |
| 22 | Protein | Amino acid sequence of the signal peptide of the precursor form of the wild-type hIL-2 polypeptide |
| 23 | Protein | Amino acid sequence of the mature form of a wild-type mouse IL-2 polypeptide |
| 24 | Protein | Amino acid sequence of the precursor form of a wild-type mouse IL-2 polypeptide (including signal peptide) |
| 25 | Protein | Amino acid sequence of wild-type HSV-TK polypeptide |
| 26 | Protein | Amino acid sequence of an HSV-TK variant polypeptide comprising 159Ile, 160Leu, 161Ala, 168Tyr, and 169Phe (“dm30”) |
| 27 | Protein | Amino acid sequence of an HSV-TK variant polypeptide comprising 159Ile, 160Phe, 161Leu, 168Phe, and 169Met (“SR39”) |
| 28 | Protein | Amino acid sequence of an HSV-TK variant polypeptide where amino acid 168 is His (“TK.007” or “HSV-TK.007”) |

### G. Examples

The following examples are provided for the purpose of illustrating certain aspects of the invention and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); i.v., intravenous(ly); i.t., intratumoral(ly); and the like.

### Example 1: Generation of Recombinant Vaccinia Virus Constructs

Select features of certain RVV constructs generated in connection with the examples provided below are summarized in Table 1, below. Each virus in Table 1 has a deletion of the J2R gene except VV10 and VV18 which have an insertional inactivation of the J2R gene. VV27, VV79, VV91-W96 and IGV-121 have the gene encoding a mouse IL-2 variant (with F76A, Y79A, L106G substitutions) which was codon optimized for expression in mouse cells. VV75 and VV101-VV103 have the gene encoding a human IL-2 variant (with F62A, Y65A, and L92G substitutions) which was codon optimized for expression in human cells.

TABLE 1

Features of Recombinant Vaccinia Virus (RVV) Constructs

| RVV Construct ID | Description | | | | | |
|---|---|---|---|---|---|---|
| | Strain | Transgene 1 | Transgene 1 location / orientation | Transgene 2 | Transgene 2 location / orientation | A34R |
| VV7 | Cop | Luc-GFP | J2R / forward | | | WT |
| VV10 | Cop | mGM-CSF | J2R / reverse | LacZ | J2R / forward | WT |
| VV16 | Cop | Luc-GFP | J2R / forward | | | K151 E |
| VV18 | Cop | mGM-CSF | J2R / reverse | LacZ | J2R / forward | K151 E |
| VV27 | Cop | mIL-2v | J2R / forward | | | K151 E |
| VV75 | Cop | hIL-2v | J2R / forward | | | K151 E |
| VV90 | Cop | | | | | K151 E |
| VV91 | Cop | mIL-2v | J2R / forward | HSVTK.0 07 | B16R partial / forward | K151 E |
| VV92 | Cop | mIL-2v | J2R / forward | HSVTK.0 07 | B16R partial / reverse | K151 E |
| VV93 | Cop | mIL-2v | J2R / forward | HSVTK.0 07 | J2R / reverse | K151 E |
| VV95 | Cop | mIL-2v | J2R / forward | HSVTK.0 07 | B 16R / forward | K151 E |
| VV96 | Cop | mIL-2v | J2R / forward | HSVTK.0 07 | B 16R / reverse | K151 E |
| VV101 | Cop | hIL-2v | J2R / forward | HSVTK.0 07 | J2R / reverse | K151 E |
| VV102 | Cop | hIL-2v | J2R / forward | HSVTK.0 07 | B16R partial / forward | K151 E |
| VV103 | Cop | hIL-2v | J2R / forward | HSVTK.0 07 | B 16R / reverse | K151 E |
| VV3 | WR | Luc-GFP | J2R / forward | | | WT |
| VV17 | WR | Luc-GFP | J2R / forward | | | K151 E |
| VV79 | WR | mIL-2v | J2R / forward | | | K151 E |
| VV94 | WR | mIL-2v | J2R / forward | HSVTK.0 07 | J2R / reverse | K151 E |
| IGV-121 | WR | mIL-2v | J2R / forward | HSVTK.0 07 | B15R-B17L / forward | K151 E |

### VV27 Construction

The virus is based on the Copenhagen strain of vaccinia and carries the gene encoding the mouse IL-2 variant under the control of a synthetic early late promoter and operator. The virus was engineered for enhanced extracellular enveloped virus (EEV) production by incorporation of a K151E substitution in the A34R gene. VV27 was constructed using a helper virus-mediated, restriction enzyme-guided, homologous recombination repair and rescue technique. First, the gene encoding mouse IL-2v (F76A, Y79A, L106G) was codon optimized for expression in mouse cells and synthesized by GeneWiz (South Plainfield, NJ). The DNA was digested with BglII/AsiSI and inserted into the Copenhagen J2R homologous recombination plasmid also digested with BglII/AsiSI. The mouse IL-2v gene and flanking left and right vaccinia homology regions were amplified by PCR to generate the homologous recombination donor fragment. BSC-40 cells were infected with Shope Fibroma Virus (SFV), a helper virus, for one hour and subsequently transfected with a mixture of the donor amplicon and purified vaccinia genomic DNA previously restriction digested within the J2R region. The parent genomic DNA originated from a Copenhagen strain vaccinia virus carrying firefly luciferase and GFP in place of the native J2R gene and a K151E mutation (substitution) within the A34R gene for enhanced EEV production. Transfected cells were incubated until significant cytopathic effects were observed and total cell lysate was harvested by 3 rounds of freezing/thawing and sonication. Lysates were serially diluted, plated on BSC-40 monolayers, and covered by agar overlay. GFP negative plaques were isolated under a fluorescent microscope over a total of three rounds of plaque purification. One plaque (KR144) was selected for intermediate amplification in BSC-40 cells in a T225 flask, prior to large scale amplification in HeLa cells in a 20-layer cell factory. The virus was purified by sucrose gradient ultracentrifugation and thoroughly characterized in quality control assays, including full genome next generation sequencing.

### VV38 Construction

The virus is based on the Copenhagen strain of vaccinia and carries the gene encoding the mouse IL-2 variant under the control of a synthetic early late promoter and operator. The virus is identical to VV27 except that it carries a wildtype A34R gene and is not engineered for enhanced EEV production. VV38 was constructed using a helper virus-mediated, restriction enzyme-guided, homologous recombination repair and rescue technique. BSC-40 cells were infected with SFV helper virus for 1-2 hours and subsequently transfected with a mixture of the donor amplicon and purified vaccinia genomic DNA previously digested with AsiSI in the J2R region. The parent genomic DNA originated from a Copenhagen strain vaccinia virus carrying firefly luciferase and GFP in place of the native J2R gene. Transfected cells were incubated until significant cytopathic effects were observed and total cell lysate was harvested by 3 rounds of freezing/thawing and sonication. Lysates were serially diluted, plated on BSC-40 monolayers, and covered by agar overlay. GFP negative plaques were isolated under a fluorescent microscope for a total of three rounds of plaque purification. One plaque (LW226) was selected for intermediate amplification in BSC-40 cells in a T225 flask, prior to large scale amplification in HeLa cells in a 20-layer cell factory. The virus was purified by sucrose gradient ultracentrifugation and thoroughly characterized in quality control assays, including full genome next generation sequencing.

### VV39 Construction

The virus is based on the Western Reserve (WR) strain of vaccinia and carries the gene encoding the mouse IL-2 variant under the control of a synthetic early late promoter and operator. VV39 was constructed using a helper virus-mediated, restriction enzyme-guided, homologous recombination repair and rescue technique. BSC-40 cells were infected with SFV helper virus for 1-2 hours and subsequently transfected with a mixture of the donor amplicon and purified vaccinia genomic DNA previously digested with AsiSI in the J2R region. The parent genomic DNA originated from a WR strain vaccinia virus carrying a luciferase-2A-GFP reporter gene cassette in place of the native J2R gene and a wild-type A34R, which is not engineered for enhanced EEV production. Transfected cells were incubated until significant cytopathic effects were observed and total cell lysate was harvested by 3 rounds of freezing/thawing and sonication. Lysates were serially diluted, plated on BSC-40 monolayers, and covered by agar overlay. GFP negative plaques were isolated under a fluorescent microscope for a total of three rounds of plaque purification. One plaque (LW228) was selected for intermediate amplification in BSC-40 cells in a T225 flask, prior to large scale amplification in HeLa cells in a 20-layer cell factory. The virus (lot #180330) was purified by sucrose gradient ultracentrifugation and thoroughly characterized in quality control assays, including full genome next generation sequencing.

### VV79 Construction

VV79, the WR strain equivalent of Copenhagen VV27, is identical to VV39 except for the addition of the A34R K151E substitution. It was constructed using helper virus mediated, homologous recombination repair and rescue techniques to insert the K151E mutation into the VV39 parental virus backbone.

### VV101 Construction

VV101 is an armed oncolytic virus based upon the Copenhagen (Cop) strain of vaccinia virus. It differs from the parental Copenhagen smallpox vaccine strain by four genetic modifications, including 1) deletion of the native vaccinia J2R (thymidine kinase) gene, 2) insertion of a human IL-2 variant (hIL-2v) expression cassette controlled by a synthetic early-late promoter within the J2R locus, 3) insertion of a herpes simplex virus (HSV) thymidine kinase variant (TK.007) expression cassette controlled by an F17 promoter within the J2R locus in the opposite orientation as the hIL-2v cassette, and 4) mutation within the viral A34R gene that introduces a lysine to glutamate substitution at position 151 of the A34 protein (K151E). VV101 was constructed using helper virus mediated, homologous recombination repair and rescue techniques. First, the gene encoding HSV TK.007 was codon optimized for expression by vaccinia virus and synthesized by Genscript. The gene was cloned downstream of an F17 promoter (P_{F17}) in a homologous recombination vector targeting the J2R region of vaccinia Copenhagen. Second, vaccinia nucleic acids were extracted from purified VV27 and transfected into Shope Fibroma Virus infected BSC-40 cells along with the HSV TK.007 / J2R homologous recombination plasmid. Following a 3-day incubation, lysates were harvested by repeated freezing and thawing. Viruses were carried through 4 rounds of plaque purification and screened for the presence of HSV-TK.007 by PCR. The virus, labelled VV93, was expanded in HeLa cells, purified by sucrose gradient centrifugation, and characterized in quality control assays, including full genome next generation sequencing. Finally, VV101 was constructed from VV93 by replacing the gene encoding mouse IL-2 variant (mIL-2v) with a gene encoding hIL-2v, optimized for expression in human, using helper virus mediated, homologous recombination repair and rescue techniques as described above. Following recombination, plaque purification and screening, VV101 was expanded in HeLa cells, purified by sucrose gradient centrifugation, and characterized in quality control assays, including full genome next generation sequencing.

### VV102 Construction

VV102 is an armed oncolytic virus based upon the Copenhagen strain of vaccinia virus. It differs from the parental Copenhagen smallpox vaccine strain by four genetic modifications, including 1) deletion of the native vaccinia J2R gene, 2) insertion of a hIL-2v expression cassette controlled by a synthetic early-late promoter within the J2R locus, 3) insertion of an HSV thymidine kinase variant (TK.007) expression cassette controlled by an F17 promoter within the B16R locus, replacing 159 bases of the native B16R gene, and 4) mutation within the viral A34R gene that introduces a lysine to glutamate substitution at position 151 of the A34 protein (K151E). VV102 was constructed using helper virus mediated, homologous recombination repair and rescue techniques. First, the gene encoding HSV TK.007 was codon optimized for expression by vaccinia virus and synthesized by Genscript. The gene was cloned downstream of an F17 promoter (P_{F17}) in a homologous recombination vector targeting the B16R region of vaccinia Copenhagen. Second, vaccinia nucleic acids were extracted from purified VV27 (described in IGNT-001) and transfected into Shope Fibroma Virus infected BSC-40 cells along with the HSV TK.007 / B16 homologous recombination plasmid. Following a 3-day incubation, lysates were harvested by repeated freezing and thawing. Viruses were carried through 4 rounds of plaque purification and screened for the presence of HSV TK.007 by PCR. The virus, labelled VV91, was expanded in HeLa cells, purified by sucrose gradient centrifugation, and characterized in quality control assays, including full genome next generation sequencing. Finally, VV102 was constructed from VV91 by replacing the gene encoding mIL-2v with a gene encoding hIL-2v, optimized for expression in human, using helper virus mediated, homologous recombination repair and rescue techniques as described above. Following recombination, plaque purification and screening, VV102 was expanded in HeLa cells, purified by sucrose gradient centrifugation, and characterized in quality control assays, including full genome next generation sequencing.

### VV103 Construction

VV103 is an armed oncolytic virus based upon the Copenhagen strain of vaccinia virus. It differs from the parental Copenhagen smallpox vaccine strain by four genetic modifications, including 1) deletion of the native vaccinia J2R gene, 2) insertion of a hIL-2v expression cassette controlled by a synthetic early-late promoter within the J2R locus, 3) insertion of an HSV thymidine kinase variant (TK.007) expression cassette controlled by an F17 promoter within the B16R locus, replacing the entire native B16R gene, and 4) mutation within the viral A34R gene that introduces a lysine to glutamate substitution at position 151 of the A34 protein (K151E). VV103 was constructed using helper virus mediated, homologous recombination repair and rescue techniques. First, the gene encoding HSV TK.007 was codon optimized for expression by vaccinia virus and synthesized by Genscript. The gene was cloned downstream of an F17 promoter (P_{F17}) in a homologous recombination vector targeting the B16R region of vaccinia Copenhagen. Second, vaccinia nucleic acids were extracted from purified VV27 (described in IGNT-001) and transfected into Shope Fibroma Virus infected BSC-40 cells along with the HSV TK.007 / B16 homologous recombination plasmid. Following a 3-day incubation, lysates were harvested by repeated freezing and thawing. Viruses were carried through 4 rounds of plaque purification and screened for the presence of HSV TK.007 by PCR. The virus, labelled VV96, was expanded in HeLa cells, purified by sucrose gradient centrifugation, and characterized in quality control assays, including full genome next generation sequencing. Finally, W103 was constructed from VV96 by replacing the gene encoding mIL-2v with a gene encoding hIL-2v, optimized for expression in human, using helper virus mediated, homologous recombination repair and rescue techniques as described above. Following recombination, plaque purification and screening, VV103 was expanded in HeLa cells, purified by sucrose gradient centrifugation, and characterized in quality control assays, including full genome next generation sequencing.

### VV94 Construction

VV94 is an armed oncolytic virus based upon the mouse-adapted Western Reserve (WR) strain of vaccinia virus. It differs from the parental WR strain by four genetic modifications, including 1) deletion of the native vaccinia J2R gene, 2) insertion of a mIL-2v expression cassette controlled by a synthetic early-late promoter within the J2R locus in the forward orientation, 3) insertion of an HSV thymidine kinase variant (TK.007) expression cassette controlled by an F17 promoter within the J2R locus in the reverse orientation, and 4) mutation within the viral A34R gene that introduces a lysine to glutamate substitution at position 151 of the A34 protein (K151E). VV94 was constructed using helper virus mediated, homologous recombination repair and rescue techniques. First, the gene encoding HSV TK.007 was codon optimized for expression by vaccinia virus and synthesized by Genscript. The gene was cloned downstream of an F17 promoter (P_{F17}) in a homologous recombination vector targeting the WR J2R region. Second, vaccinia nucleic acids were extracted from purified VV79 and transfected into Shope Fibroma Virus infected BSC-40 cells along with the HSVTK.007 / J2R homologous recombination amplicon. Following a 3-day incubation, lysates were harvested by repeated freezing and thawing. Viruses were carried through 4 rounds of plaque purification and screened for the presence of HSV- TK.007 by PCR. The virus, labelled VV94, was expanded first in BSC-40 cells, then in HeLa cells, purified by sucrose gradient centrifugation, and characterized in quality control assays, including full genome next generation sequencing.

### IGV-121 Construction

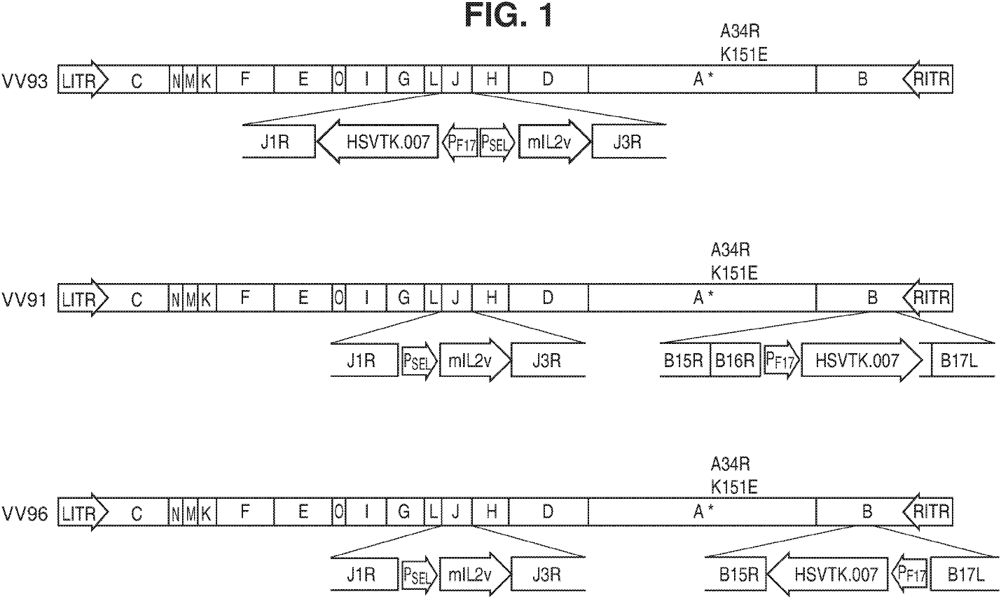

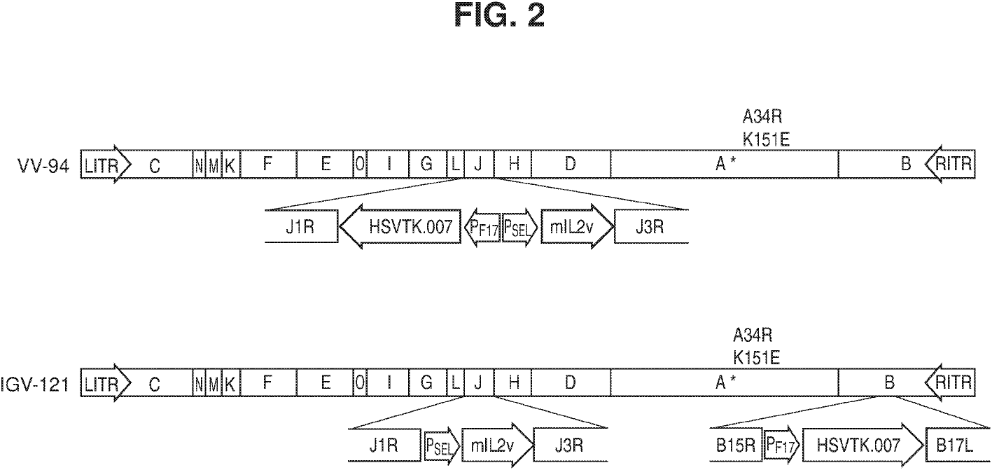

IGV-121 is an armed oncolytic virus based upon the mouse-adapted WR strain of vaccinia virus. It differs from the parental WR strain by four genetic modifications, including 1) deletion of the native vaccinia J2R gene, 2) insertion of a mIL-2v variant expression cassette controlled by a synthetic early-late promoter within the J2R locus, 3) insertion of an HSV thymidine kinase variant (TK.007) expression cassette controlled by an F17 promoter in the intergenic region between B15R (also known as WR197) and B17L (WR198), and 4) mutation within the viral A34R gene that introduces a lysine to glutamate substitution at position 151 of the A34 protein (K151E). IGV-121 was constructed using helper virus mediated, homologous recombination repair and rescue techniques. First, the gene encoding HSV TK.007 was codon optimized for expression by vaccinia virus and synthesized by Genscript. The gene was cloned downstream of an F17 promoter (P_{F17}) in a homologous recombination vector targeting the intergenic region between B15R and B17L of vaccinia WR strain. Second, vaccinia nucleic acids were extracted from purified VV79 (WR strain with J2R replaced by mouse IL-2v and A34R K151E mutation) and transfected into Shope Fibroma Virus infected Vero-B4 cells along with the HSV TK.007 / B15R-B17L homologous recombination plasmid. Following a 2-day incubation, lysates were harvested by repeated freezing, thawing, and sonication. Viruses were carried through 3 rounds of plaque purification on BSC-40 cells. The virus, labelled IGV-121, was expanded in HeLa S3 cells, purified by sucrose gradient centrifugation, and characterized in quality control assays, including full genome next generation sequencing. FIG. 1. Provides schematic representation of full genomes for VV91, VV93, and VV96, FIG. 2. provides schematic representation of full genomes for VV94 and IGV-121, and FIG. 3. Provides schematic representation of full genomes for VV101-VV103.

### Example 2: Demonstration Oƒ IL-2v Expression From Recombinant Vaccinia Viruses in Virus- Infected Cells by Western Blot

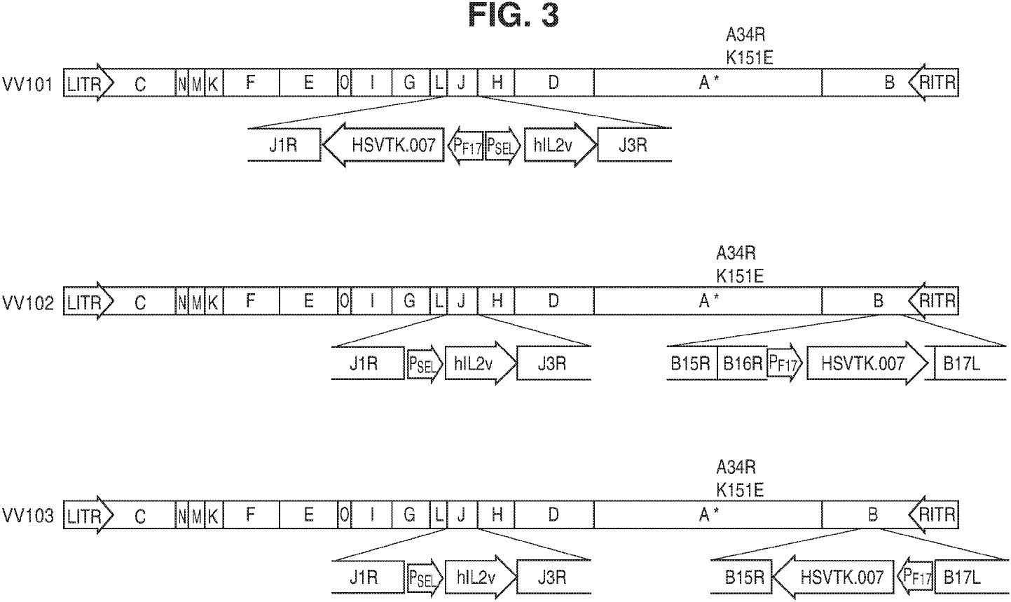

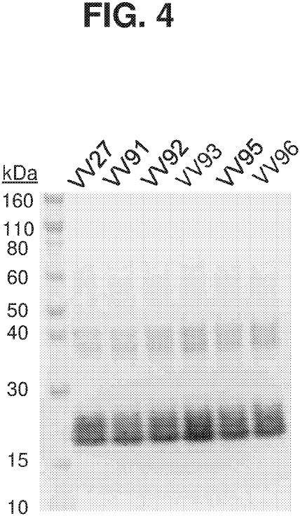

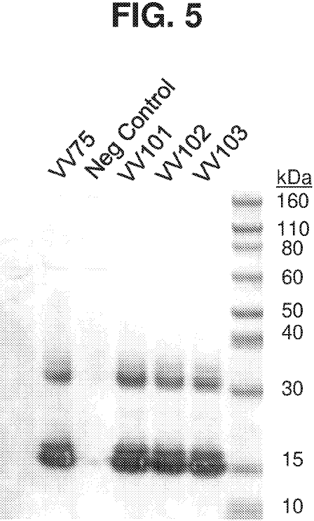

HeLa cells were plated at 6e5 cells/well in 2 mL of culture media in 6-well plates and after approx. 24 hr in culture infected with virus at MOI = 3 for 24 hr. Cells from each well were subsequently harvested and lysed in 200 µL Laemmli buffer then diluted 1:1 with milliQ water. 12 µL of sample was prepared to a final volume of 20 µL in Tris-buffered saline (TBS) containing Reducing Agent and 1x NuPage LDS sample buffer prior to incubation at 95_{□}DC for 5 min and loading on a NuPage 4-12% Bis-Tris gel. Gel electrophoresis with 1xMES running buffer was performed at 200 V for 30 min. Proteins were transferred to a PVDF membrane using an iBlot device and Western Blot was performed using an iBlot device. For detection of mIL-2v the following antibodies were used - anti-IL-2 primary antibody (Abcam, ab11510) at 1:2000 dilution, goat anti-rat IgG-HRP secondary antibody (Invitrogen, #629526) at 1:1000 dilution. For detection of hIL-2v the following antibodies were used - anti-IL-2 primary antibody (Novus Biologicals, NBP2-16948) at 1:500 dilution, mouse anti-rabbit IgG-HRP secondary antibody (Pierce, #31460) at 1:2000 dilution. TMB substrate was subsequently added to the membrane to visualize bands. Membrane was rinsed with water, dried and scanned. Results of mIL-2v expression analysis following infection of cells with recombinant oncolytic vaccinia viruses are provided in FIG. 4. Results of hIL-2v expression analysis following infection of cells with recombinant oncolytic vaccinia viruses are provided in FIG. 5.

### Example 3: Demonstration Oƒ HSV TK.007 Expression From Recombinant Vaccinia Viruses in Virus-Inƒected Cells by RT-QPCR

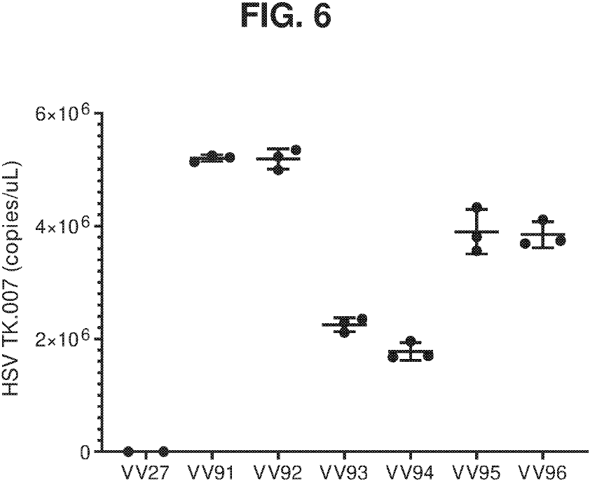

HeLa cells were plated at 7e4 cells/well in 2 mL of culture media in 6-well plates and after approx. 72 hr in culture infected with virus at MOI = 3 for 18 hr. Cells from each well were subsequently harvested and processed for RNA extraction using the RNeasy Plus Universal Mini Kit (Qiagen, #73404). 500 ng total RNA was reverse transcribed using the High Capacity cDNA Reverse Transcription Kit (applied Biosystems, #4368814). cDNA was diluted 1:10 prior to use in qPCR to analyze HSV TK.007 mRNA expression levels using primers and probes specific for the HSV TK.007 transgene encoded in the recombinant viruses and PrimeTime Gene Expression Master Mix (IDT, #1055772). PCR was conducted on a ViiA7 instrument (Applied Biosystems). Plasmid DNA containing the HSV TK.007 cDNA sequence was used as a standard and copies/µL in each test sample determined from the standard curve. Results of HSV TK.007 expression analysis following infection of cells with recombinant oncolytic vaccinia viruses are provided in FIG. 6.

### Example 4: Recombinant Oncolytic Vaccinia Virus Activity in MC38 Tumor-Bearing C57BL/6 Mice (Cop Viruses Expressing mIL-2v)

Female C57BL/6 mice (8-10 weeks old) were implanted subcutaneously (SC) on the right upper rear flank with 5e5 MC38 tumor cells. MC38 is a murine colon adenocarcinoma cell line. See, e.g., Cancer Research (1975) vol. 35, pp. 2434-2439. Eleven days after tumor cell implantation, mice were randomized based on tumor volume into separate treatment groups (average tumor volume per group ~50 mm³; N=18 /group). On day 12 post-implantation, tumors were directly injected with 60 □L vehicle (30 mM Tris, 10% sucrose, pH 8.0) or 60 □L vehicle containing 5e7 plaque forming units (pfu) of recombinant Copenhagen (Cop) vaccinia virus variant. Tumor-bearing mice were observed daily, and both tumor volumes and body weights measured bi-weekly until mice were humanely sacrificed either due to i) tumor volume surpassing 1400 mm³, ii) ≥ 20% body weight loss, or iii) severely diminished health status. Groups of mice were treated as follows:
Group i) vehicle only;
Group ii) VV16: Cop vaccinia virus carrying the A34R-K151E mutation (amino acid substitution) and armed with a Luciferase and green fluorescent protein (Luc-2A-GFP) dual reporter cassette;
Group iii) VV27: Cop vaccinia virus carrying the A34R-K151E substitution and armed with a mIL-2v transgene;
Group iv) VV91: Cop vaccinia virus carrying the A34R-K151E substitution, armed with a mIL-2v transgene, and encoding HSV TK.007 (B16R insertion, forward orientation);
Group v) VV93: Cop vaccinia virus carrying the A34R-K151E substitution, armed with a murine mIL-2v) transgene, and encoding HSV TK.007 (J2R insertion, reverse orientation); or
Group vi) VV96: Cop vaccinia virus carrying the A34R-K151E substitution, armed with a mIL-2v transgene, and encoding HSV TK.007 (B16R insertion, reverse orientation).

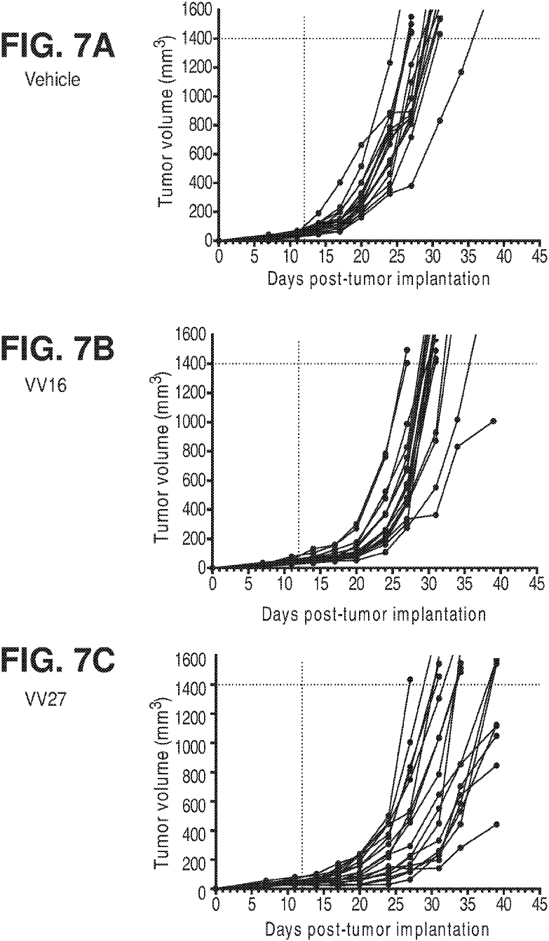

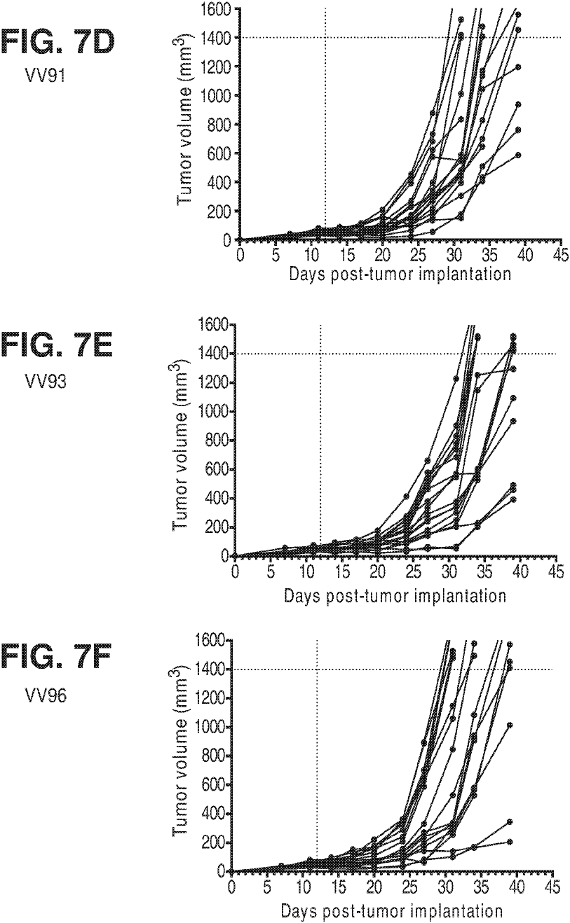

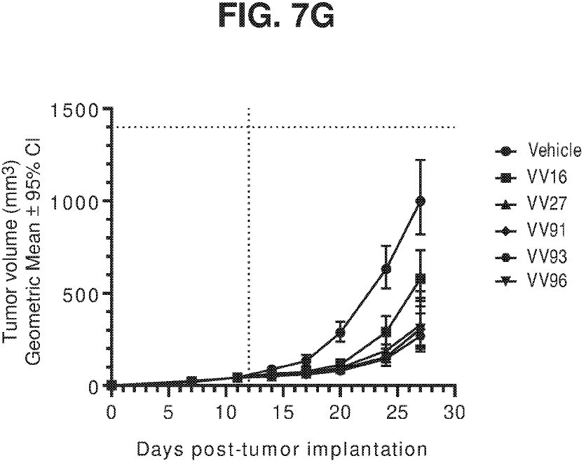

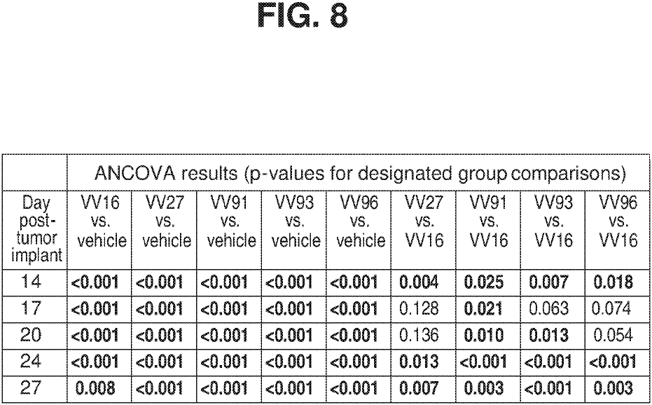

Comparisons between the tumor growth profiles of groups (i) - (vi) (FIGS. 7A-7G) revealed that all test viruses produced a statistically significant inhibitory effect on tumor growth over multiple consecutive days, all of the mIL-2v-armed Cop vaccinia viruses (VV27, VV91, VV93, and VV96) produced a statistically significant inhibitory effect on tumor growth over multiple consecutive days compared to control virus (VV16) (FIG. 8, ANCOVA results), and that there were no statistically significant differences observed when comparing VV27 (mIL-2v only) to either VV91, VV93, or VV96 (each with mIL-2v and HSV TK.007).

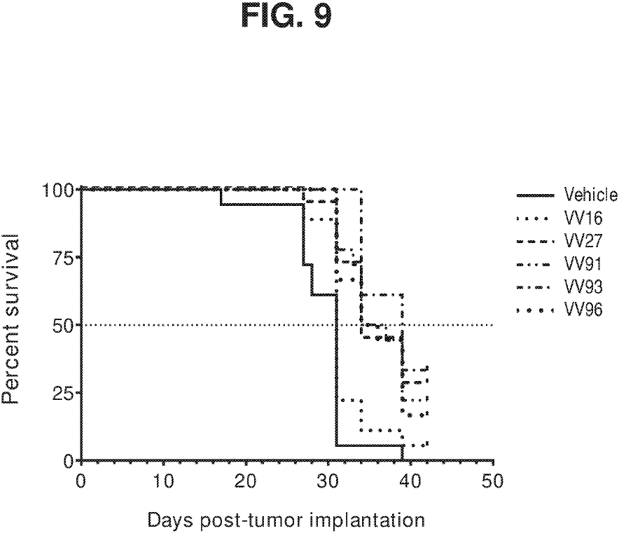

Survival of animals in each treatment group (N=18 /group) was also assessed up through day 41 post-tumor implantation (FIG. 9). The unarmed vaccinia control (VV16) did not significantly improve survival over vehicle control (Log rank/Mantel-Cox test, p=0.133). However, mice treated with armed vaccinia viral variants VV27, VV91, VV93, and VV96 showed a statistically significant mean survival advantage over the reporter transgene-armed vaccinia control (VV16) treatment group (Log rank/Mantel-Cox test, p=0.009, 0.006, <0.0001, and 0.013 respectively).

In addition to monitoring tumor growth inhibition and survival, sera were collected from tumor-bearing mice 24 hr and 48 hr after injection with vehicle or recombinant Cop vaccinia virus to assess circulating IL-2 levels. Circulating IL-2 levels in sera collected from each treatment group 24 hr and 48 hr after receiving intratumoral injections were quantified by ELISA (FIG. 10). Measurable levels of IL-2 were detected in the serum from most animals treated with the mIL-2v-armed Cop vaccinia virus variants (VV27, VV91, VV93, and VV96), while background levels of IL-2 were seen in any animal from the vehicle or other Cop vaccinia virus (VV16) groups. This latter result indicates that intratumoral injection of Cop vaccinia viruses lacking the mIL-2v transgene, at least at the tested dose levels, was insufficient to induce increased circulating IL-2 levels in the sera of treated animals. Thus, elevated levels seen in the sera of mice treated with the mIL-2v-armed Cop vaccinia virus should be indicative of transgene-mediated expression following intratumoral injection.

### Example 5: mIL-2v-armed Vaccinia Virus Activity in Lewis Lung Carcinoma (LLC) Tumor-Bearing C57BL/6 Mice (Cop Viruses Expressing mIL-2v)

Female C57BL/6 mice (8-10 weeks old) were implanted subcutaneously (SC) on the left upper rear flank with le5 LLC tumor cells. Twelve days after tumor cell implantation, mice were randomized based on tumor volume into separate treatment groups (average tumor volume per group ~50 mm³; N=20 /group). On day 13 post-implantation, tumors were directly injected with 60 □L vehicle (30 mM Tris, 10% sucrose, pH 8.0) or 60 □L vehicle containing 5e7 plaque forming units (pfu) of recombinant Copenhagen (Cop) vaccinia virus variant. Tumor-bearing mice were observed daily, and both tumor volumes and body weights measured bi-weekly until mice were humanely sacrificed either due to i) tumor volume surpassing 1400 mm³, ii) ≥ 20% body weight loss, or iii) severely diminished health status. Groups of mice were treated as follows:
Group i) vehicle only;
Group ii) VV16: Cop vaccinia virus carrying the A34R-K151E mutation (amino acid substitution) and armed with a Luciferase and green fluorescent protein (Luc-2A-GFP) dual reporter cassette;
Group iii) VV27: Cop vaccinia virus carrying the A34R-K151E substitution and armed with a mIL-2v transgene;
Group iv) VV91: Cop vaccinia virus carrying the A34R-K151E substitution, armed with a mIL-2v transgene, and encoding HSV TK.007 (B16R insertion, forward orientation);
Group v) VV93: Cop vaccinia virus carrying the A34R-K151E substitution, armed with a mIL-2v transgene, and encoding HSV TK.007 (J2R insertion, reverse orientation); or
Group vi) VV96: Cop vaccinia virus carrying the A34R-K151E substitution, armed with a mIL-2v transgene, and encoding HSV TK.007 (B16R insertion, reverse orientation).

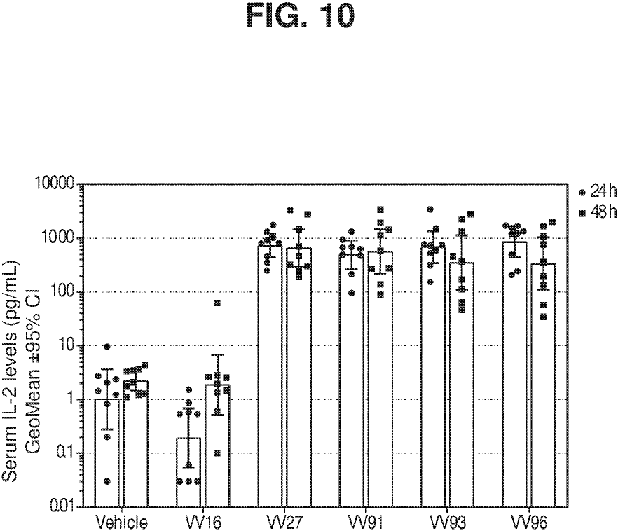

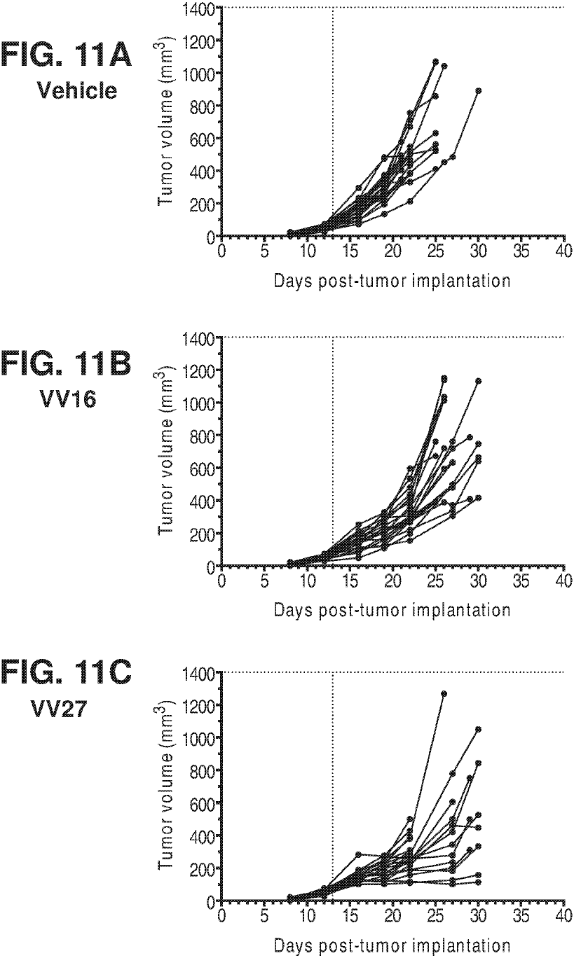

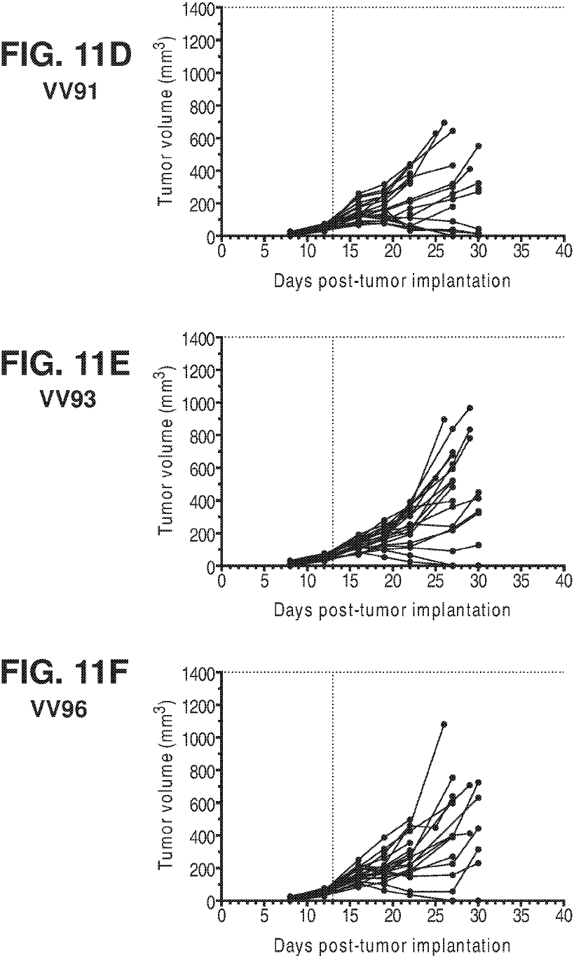

Comparisons between the tumor growth profiles of groups (i) - (vi) (FIGS. 11A-11F) revealed that all test viruses produced an inhibitory effect on tumor growth, with the mIL-2v-armed Cop vaccinia viruses (VV27, W91, VV93, and VV96) produced a more striking inhibitory effect on tumor growth compared to control virus (VV16). Many animals on study were humanely sacrificed due to tumor ulcerations and associated diminished health status limiting statistical analyses of tumor growth inhibition associated with different virus variants. However, analysis of individual animals demonstrated that 7/20, 2/20, and 1/20 tumors were either <50 mm³ or completely regressed by day 30 post-implant following treatment with W91, VV93, or VV96, respectively, with no small tumors or complete regressions observed in other treatment groups.

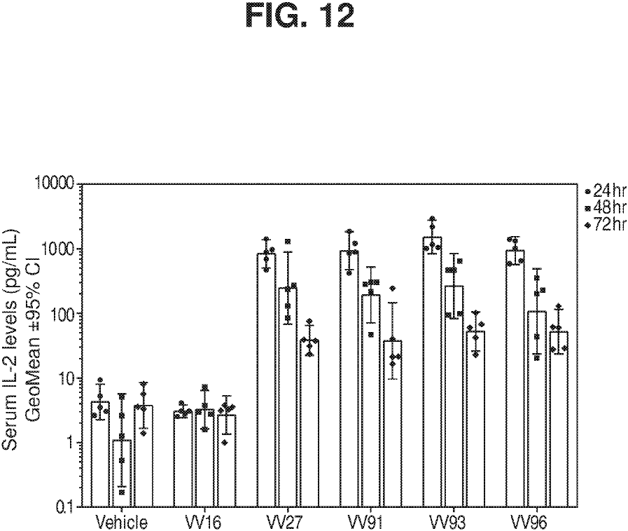

In addition to monitoring tumor growth inhibition and survival, sera were collected from tumor-bearing mice 24, 48, and 72 hr after injection with vehicle or recombinant Cop vaccinia virus to assess circulating IL-2 levels. Circulating IL-2 levels in sera collected from each treatment group at these timepoints after receiving intratumoral injections were quantified by ELISA (FIG. 12). Measurable levels of IL-2 were detected in the serum from most animals treated with the mIL-2v-armed Cop vaccinia virus variants (VV27, VV91, VV93, and VV96), while background levels of IL-2 were seen in any animal from the vehicle or other Cop vaccinia virus (VV16) groups. This latter result indicates that intratumoral injection of Cop vaccinia viruses lacking the mIL-2v transgene, at least at the tested dose levels, was insufficient to induce increased circulating IL-2 levels in the sera of treated animals. Thus, elevated levels seen in the sera of mice treated with the mIL-2v-armed Cop vaccinia virus should be indicative of transgene-mediated expression following intratumoral injection.

### Example 6: Single IV Virotherapy Using Recombinant Oncolytic Vaccinia Virus in MC38 Tumor-Bearing C57BL/6 Mice (WR Viruses Expressing mIL-2v)

C57BL/6 female mice were implanted SC on the left flank with 5e5 MC38 tumor cells. Ten days after tumor cell implantation, mice were randomized based on tumor volume into separate treatment groups (average tumor volume per group ~50 mm³; N=15 /group). On day 11 post-tumor cell implantation, mice were injected IV with 100 □L of vehicle (30 mM Tris, 10% sucrose, pH8.0) or 100 □L of vehicle containing 5e7 pfu recombinant WR vaccinia virus. Tumor-bearing mice were observed daily, and both tumor volume and body weight were measured bi-weekly until mice were humanely sacrificed either due to i) tumor volume surpassing 1400 mm³, ii) ≥ 20% body weight loss, iii) severely diminished health status or iv) study termination.

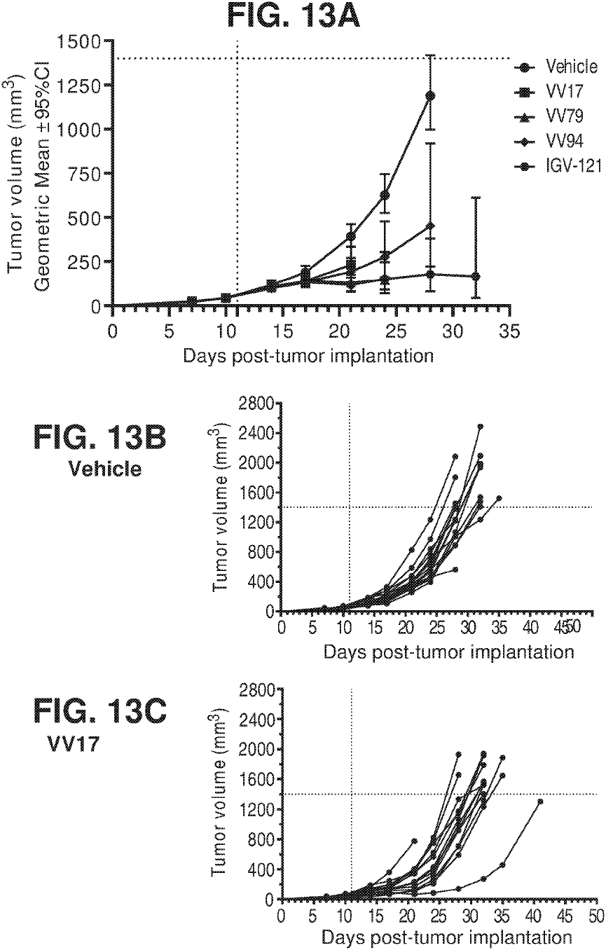

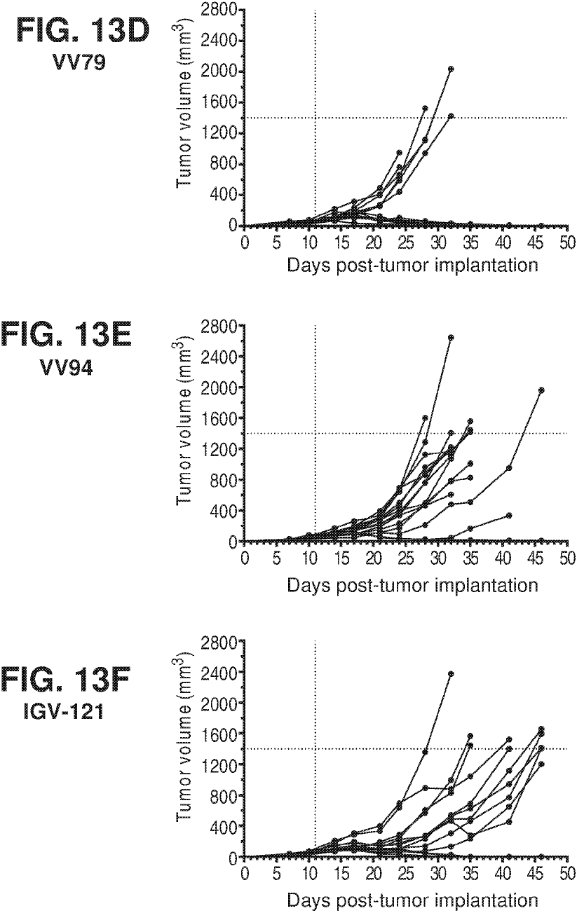

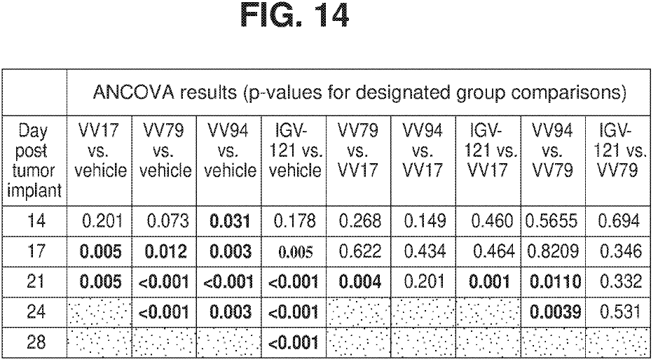

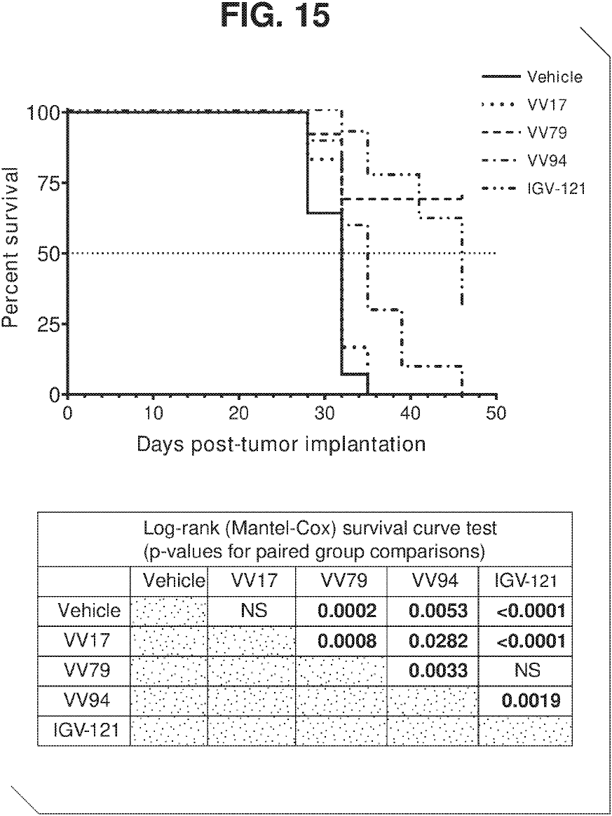

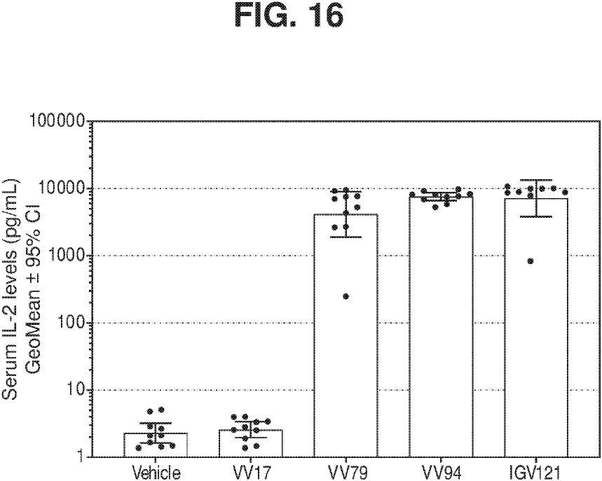

Analysis of tumor growth profiles, shown as group averages for each test virus (FIG. 13A) or as individual mice within each test group (FIGS. 13B-13F), revealed an important finding. IV administration of mIL-2v transgene-armed WR viruses (encoding mIL-2v alone or with HSV TK.007) led to statistically significant inhibition of MC38 tumor growth compared to vehicle and reporter transgene-armed WR virus (VV17) treatment. There was no statistically significant difference between tumor growth inhibition induced by VV79 and IGV-121, however there was a statistically significant difference detected between VV79 and VV94 (FIG. 14, ANCOVA results).

Survival results for the same test viruses showed very similar outcomes as those reported above for tumor growth inhibition. This included statistically superior group survival associated with mIL-2v transgene-armed WR viruses in the presence or absence of the HSV TK.007 compared to the corresponding Luc-GFP reporter-armed WR virus (FIG. 15). Overall, IV delivery of mIL-2v transgene-armed WR virus variants proved to be an effective anti-tumor therapy in the MC38 SC tumor model and demonstrated the potency of a single therapeutic administration of virus.

Sera were also collected from MC38 tumor-bearing mice in each test group at 72 hr (day 14) after the IV virus dose for assessment of circulating IL-2 levels. Consistent with other studies where mIL-2v transgene-armed viruses were tested, elevated and statistically significant serum levels of IL-2 were detected in all test groups where mIL-2v transgene-armed WR virus was administered (FIG. 16).

### Example 7: Single IV Virotherapy Using Recombinant Oncolytic Vaccinia Virus in LLC Tumor-Bearing C57BL/6 Mice (WR viruses Expressing mIL-2v)

In this set of experiments, C57BL/6 female mice were implanted SC on the right flank with le5 LLC tumor cells. Twelve days after tumor cell implantation, mice were randomized based on tumor volume into separate treatment groups (average tumor volume per group ~50 mm³; N=20 /group). On day 14 mice were injected IV with 100 □L of vehicle (30 mM Tris, 10% sucrose, pH8.0) or 100 □L of vehicle containing 5e7 pfu recombinant WR vaccinia virus variants. Tumor-bearing mice were observed daily, and both tumor volume and body weight were measured bi-weekly until mice were humanely sacrificed either due to i) tumor volume surpassing 2000 mm³, ii) ≥ 20% body weight loss, iii) severely diminished health status or iv) study termination.

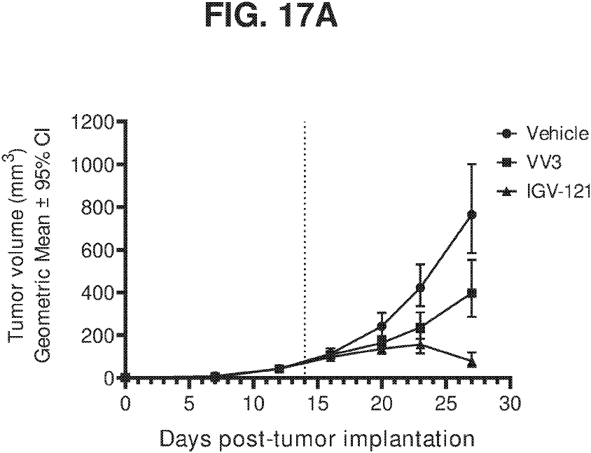

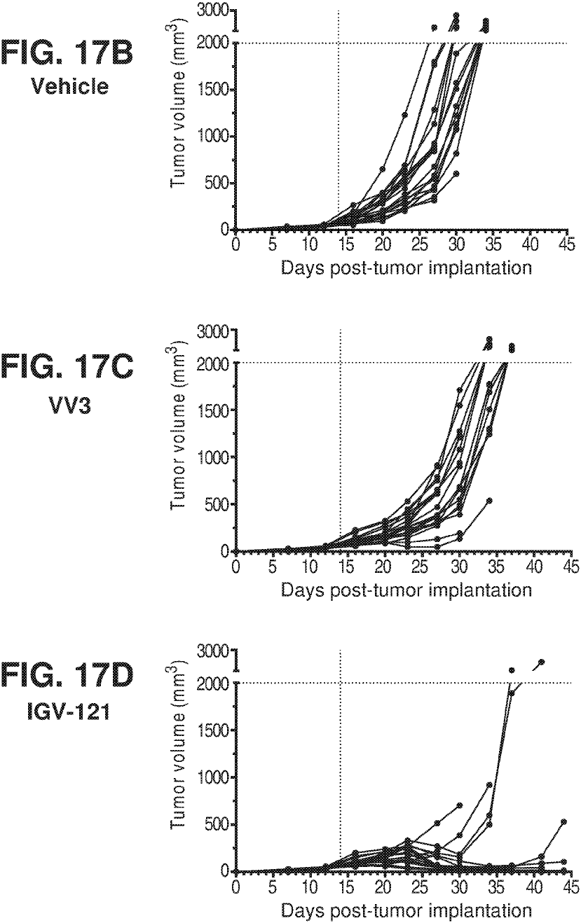

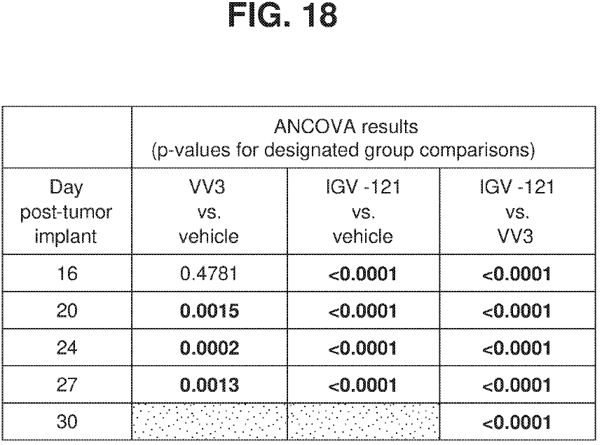

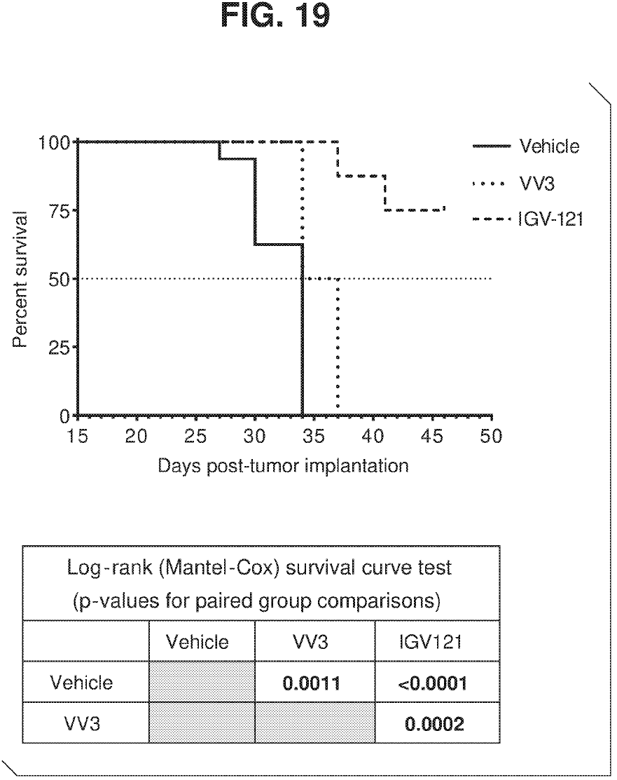

Analysis of tumor growth profiles, shown as group averages for each test virus (FIG. 17A) or as individual mice within each test group (FIGS. 17B-17D) demonstrated that IV administration of the mIL-2v transgene-armed WR viruses encoding HSV TK.007 and the A34R-K151E mutation (IGV-121) led to statistically significant inhibition of LLC tumor growth compared to reporter transgene-armed WR virus treatment (FIG. 18, ANCOVA results).

Survival results for the same test viruses showed very similar outcomes as those reported above for tumor growth inhibition. This included statistically superior group survival associated with mIL-2v and HSV TK.007 transgene-armed WR viruses compared to the corresponding Luc-GFP reporter-armed WR viruses (FIG. 19). Overall, IV delivery of the mIL-2v transgene-armed WR virus variant proved to be an effective anti-tumor therapy in the LLC SC tumor model and demonstrated the potency of a single therapeutic administration of virus.

### Example 8: Recombinant Oncolytic Vaccinia Virus Activity in MC38 Tumor-Bearing C57BL/6 Mice (Cop Viruses Expressing mIL-2v or hIL-2v)

Female C57BL/6 mice (8-10 weeks old) were implanted subcutaneously (SC) on the right upper rear flank with 5e5 MC38 tumor cells. Ten days after tumor cell implantation, mice were randomized based on tumor volume into separate treatment groups (average tumor volume per group ~50 mm³; N=20 /group). On day 11 post-implantation, tumors were directly injected with 60 □L vehicle (30 mM Tris, 10% sucrose, pH 8.0) or 60 □L vehicle containing either 5e7 or 2e8 plaque forming units (pfu) of recombinant Copenhagen (Cop) vaccinia virus variant. Tumor-bearing mice were observed daily, and both tumor volumes and body weights measured bi-weekly until mice were humanely sacrificed either due to i) tumor volume surpassing 1400 mm³, ii) ≥ 20% body weight loss, or iii) severely diminished health status. Groups of mice were treated as follows:
Group i) vehicle only;
Group ii) VV7 at 2e8 pfu dose level: Cop vaccinia virus armed with a Luciferase and green fluorescent protein (Luc-2A-GFP) dual reporter cassette;
Group iii) VV91 at 5e7 pfu dose level: Cop vaccinia virus carrying the A34R-K151E substitution, armed with a murine interleukin 2 variant (mIL-2v) transgene, and encoding HSV TK.007 (B16R insertion, forward orientation);
Group iv) VV91 at 2e8 pfu dose level: Cop vaccinia virus carrying the A34R-K151E substitution, armed with a murine interleukin 2 variant (mIL-2v) transgene, and encoding HSV TK.007 (B16R insertion, forward orientation);
Group v) VV102: at 5e7 pfu dose level: Cop vaccinia virus carrying the A34R-K151E substitution, armed with a human interleukin 2 variant (hIL-2v) transgene, and encoding HSV TK.007 (B16R insertion, forward orientation);
Group vi) VV102 at 2e8 pfu dose level: Cop vaccinia virus carrying the A34R-K151E substitution, armed with a human interleukin 2 variant (hIL-2v) transgene, and encoding HSV TK.007 (B16R insertion, forward orientation);
Group vii) VV10 at 5e7 pfu dose level: Cop vaccinia virus armed with mouse GM-CSF and LacZ reporter transgenes; or
Group viii) VV10 at 2e8 pfu dose level: Cop vaccinia virus armed with mouse GM-CSF and LacZ reporter transgenes;

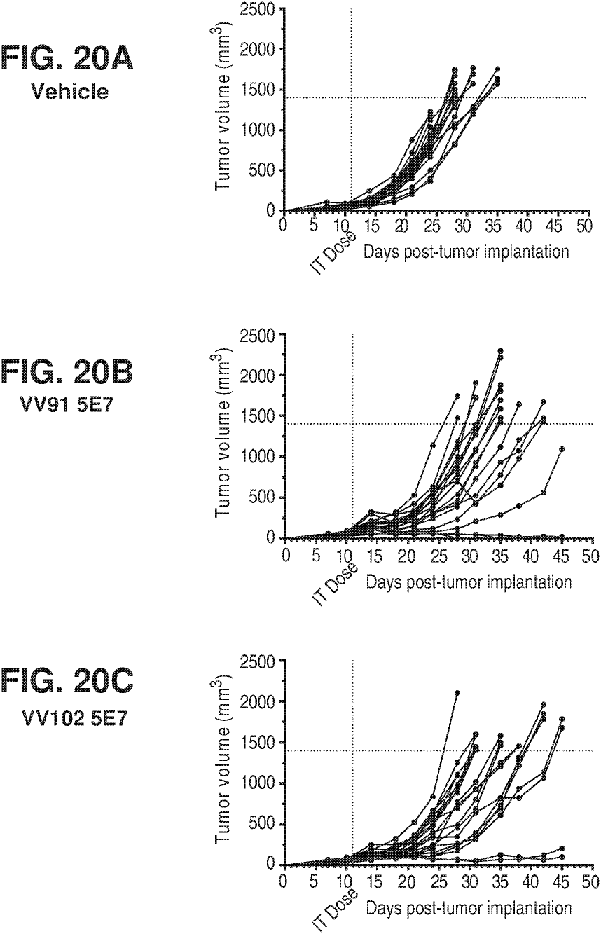

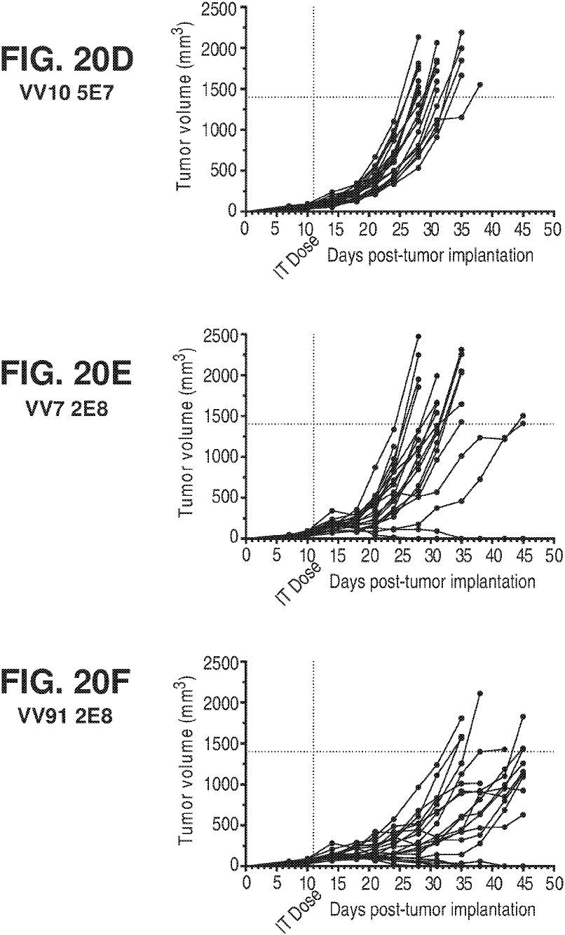

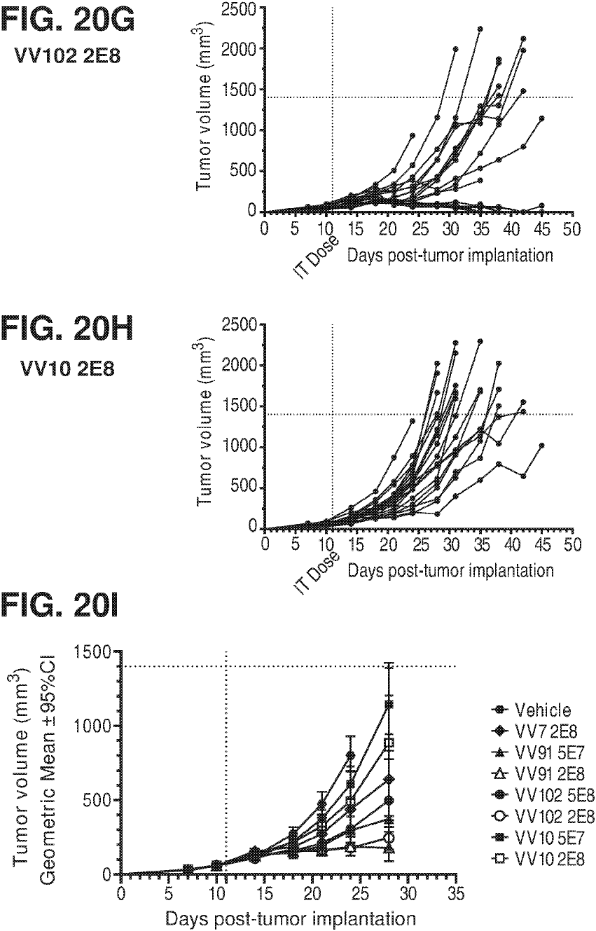

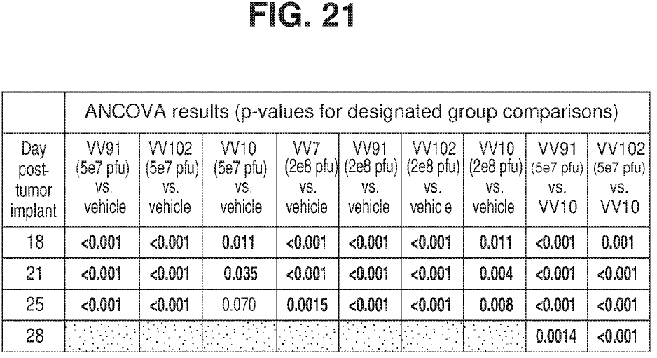

Comparisons between the tumor growth profiles of groups (i) - (viii) (FIGS. 20A-20I) revealed that all test viruses produced a statistically significant inhibitory effect on tumor growth over multiple consecutive days, and that the mouse and human IL-2v-armed Cop vaccinia viruses (W91 and VV102, respectively) produced a statistically significant inhibitory effect on tumor growth over multiple consecutive days compared to mouse GM-CSF-armed Cop vaccinia virus (VV10) (FIG. 21, ANCOVA results). There were no statistically significant differences observed when comparing tumor growth inhibition effects induced by VV91 (mIL-2v and HSV TK\.007) to VV102, (hIL-2v and HSV TK\.007).

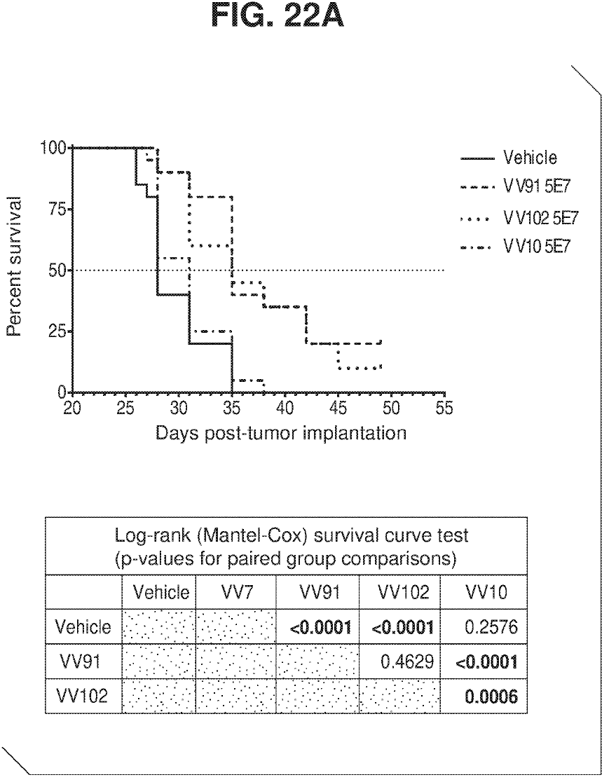

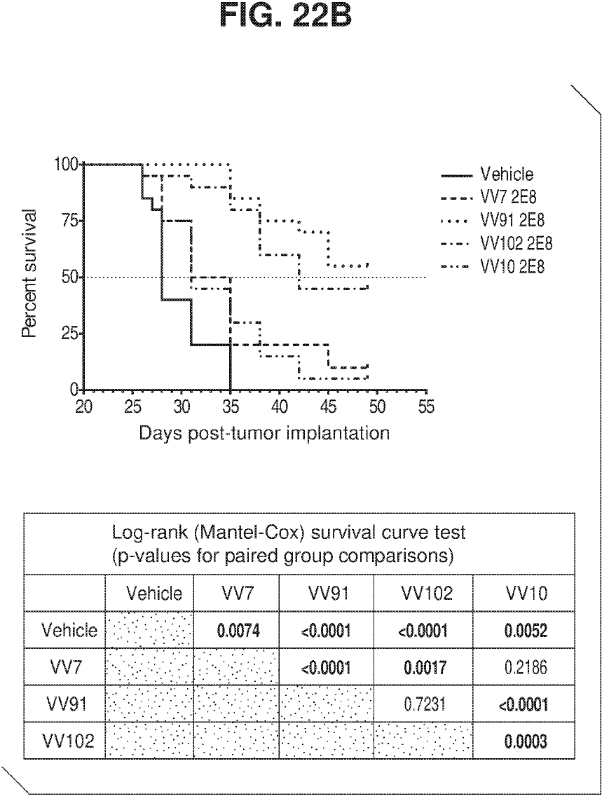

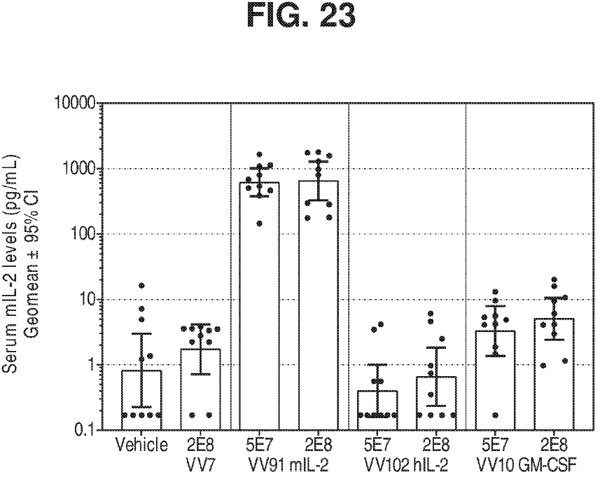

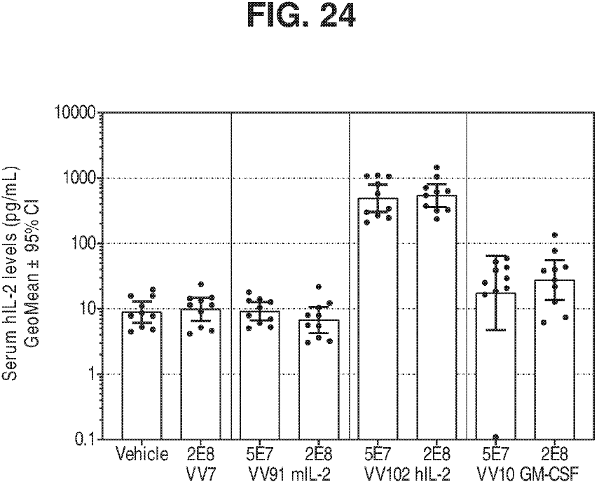

Survival of animals in each treatment group (N=20 /group) was also assessed up through day 42 post-tumor implantation (FIG. 22). In this case, mice treated with both VV91 and VV102 showed a statistically significant mean survival advantage over vehicle, VV7, and VV10 treatment groups (see table in FIG. 22 for P values from Log rank/Mantel-Cox test).

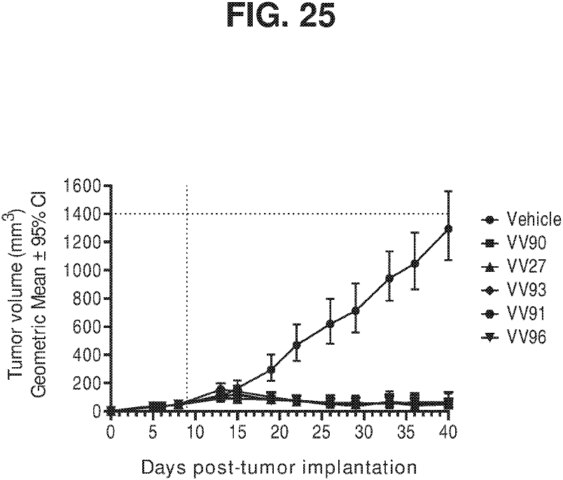

In addition to monitoring tumor growth inhibition and survival, sera were collected from tumor-bearing mice 24 hr after injection with vehicle or recombinant Cop vaccinia virus to assess circulating IL-2 levels. Circulating mouse IL-2 and human IL-2 levels in sera collected from each treatment group 24 hr after receiving intratumoral injections were quantified by ELISA (FIG. 23 and FIG. 24, respectively). Measurable levels of IL-2 were detected in the serum from most animals treated with the IL-2v-armed Cop vaccinia virus variants (VV91, and VV102), while background levels of IL-2 were seen in any animal from the vehicle or other Cop vaccinia virus (VV7 and, W10) groups. Notably, significantly elevated levels of mouse IL-2 ere only detected in serum of mice receiving mIL-2v expressing virus (VV91) and significantly elevated levels of human IL-2 were only detected in serum of mice receiving hIL-2v expressing virus (VV102). Thus, elevated levels seen in the sera of mice treated with the IL-2v-armed Cop vaccinia virus should be indicative of transgene-mediated expression following intratumoral injection.

### Example 9: Recombinant Oncolytic Vaccinia Virus Activity in HCT-116 Tumor-Bearing Nude Mice (Cop Viruses Expressing hIL-2v)

Nude female mice were implanted SC on the right flank with 5e6 HCT-116 tumor cells. Eight days after tumor cell implantation, mice were randomized based on tumor volume into separate treatment groups (average tumor volume per group ~50 mm³; N=20 /group). On day 9 post-tumor cell implantation, mice were injected IV with 100 □L of vehicle only or vehicle containing a suboptimal dose (3e5 pfu) of recombinant oncolytic Cop vaccinia virus. Tumor-bearing mice were observed daily, and both tumor volume and body weight were measured bi-weekly until mice were humanely sacrificed either due to i) tumor volume surpassing 1400 mm³, ii) ≥ 20% body weight loss, iii) severely diminished health status, or iv) study termination. Groups of mice were treated as follows:
Group i) vehicle only;
Group ii) VV90: Cop vaccinia virus carrying the A34R-K151E mutation (amino acid substitution) with no transgene inserted into the deleted J2R gene region;
Group iii) VV27: Cop vaccinia virus carrying the A34R-K151E substitution and armed with a murine interleukin 2 variant (mIL-2v) transgene (VV27);
Group iv) VV91: Cop vaccinia virus carrying the A34R-K151E substitution, armed with a murine interleukin 2 variant (mIL-2v) transgene, and encoding HSV TK.007 (B16R insertion, forward orientation);
Group v) VV93: Cop vaccinia virus carrying the A34R-K151E substitution, armed with a murine interleukin 2 variant (mIL-2v) transgene, and encoding HSV TK.007 (J2R insertion, reverse orientation); or
Group vi) VV96: Cop vaccinia virus carrying the A34R-K151E substitution, armed with a murine interleukin 2 variant (mIL-2v) transgene, and encoding HSV TK.007 (B16R insertion, reverse orientation).

Comparisons between the tumor growth profiles of groups (i) - (vi) (FIG. 25) revealed that all test viruses produced a statistically significant inhibitory effect on tumor growth over multiple consecutive days in the human xenograft tumors.

In embodiments that refer to a method of treatment as described herein, such embodiments are also further embodiments for use in that treatment, or alternatively for the manufacture of a medicament for use in that treatment.

While the present invention has been described with reference to the specific embodiments thereof, it should be understood by those skilled in the art that various changes may be made, and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A replication-competent recombinant oncolytic vaccinia virus (RVV) which comprises modifications in the viral genome relative to the corresponding wild-type virus, wherein the modifications comprises, in the viral genome: (1) an inserted nucleotide sequence encoding an immunostimulatory cytokine polypeptide; and (2) an inserted nucleotide sequence encoding a heterologous thymidine kinase (TK) polypeptide.

2. The RVV of claim 1, wherein the RVV comprises a further modification that renders the vaccinia TK deficient.

3. The RVV of claim 2, wherein the further modification results in lack of J2R expression and/or function.

4. The RVV of claim 2, wherein the RVV is a Copenhagen strain vaccinia virus.

5. The RVV of claim 2, wherein the RVV is a WR strain vaccinia virus.

6. The RW of any one of claims 1-5, wherein the immunostimulatory cytokine polypeptide is an interleukin-2 (IL-2) polypeptide.

7. The RVV of any one of claims 1-6, wherein the immunostimulatory cytokine polypeptide is a wild-type IL-2 polypeptide.

8. The RVV of any one of claims 1-6, wherein the immunostimulatory cytokine polypeptide is a variant IL-2 (IL-2v) polypeptide having reduced undesirable biological activity as compared to the corresponding wild-type IL-2 polypeptide.

9. The RVV of any one of claims 1-8, wherein the heterologous TK polypeptide is an HSV-TK polypeptide.

10. The RW of any one of claims 1-9, wherein the RW comprises an A34R gene comprising a K151E substitution.

11. The RVV of any one of claims 8-10, wherein the IL-2v polypeptide comprises substitutions of one or more of F42, Y45, and L72, based on the amino acid numbering of the IL-2 amino acid sequence of SEQ ID NO: 1.

12. The RVV of any one of claims 8-11, wherein the IL-2v polypeptide comprises substitution an F42L, F42A, F42G, F42S, F42T, F42Q, F42E, F42D, F42R, or F42K substitution, based on the amino acid numbering of the IL-2 amino acid sequence of SEQ ID NO: 1.

13. The RVV of any one of claims 8-12, wherein the IL-2v polypeptide comprises a Y45A, Y45G, Y45S, Y45T, Y45Q, Y45E, Y45N, Y45D, Y45R, or Y45K substitution, based on the amino acid numbering of the IL-2 amino acid sequence of SEQ ID NO: 1.

14. The RVV of any one of claims 8-13, wherein the IL-2v polypeptide comprises a L72G, L72A, L72S, L72T, L72Q, L72E, L72N, L72R, or L72K substitution, based on the amino acid numbering of the IL-2 amino acid sequence of SEQ ID NO: 1.

15. The RVV of any one of claims 8-14, wherein the IL-2v polypeptide comprises a F42A, Y45A, and L72G substitutions, based on the amino acid numbering of the IL-2 amino acid sequence of SEQ ID NO: 1.

16. The RW of any one of claims 8-14, wherein the IL-2v polypeptide-encoding nucleotide sequence is operably linked to a regulatable promoter.

17. The RVV of claim 16, wherein the regulatable promoter is regulated by tetracycline or a tetracycline analog or derivative.

18. The RVV of any one of claims 1-17, wherein heterologous TK polypeptide is capable of catalyzing phosphorylation of deoxyguanosine.

19. The RVV of any one of claims 1-18, wherein the heterologous TK polypeptide is a variant herpes simplex virus (HSV) TK polypeptide.

20. The RVV of claim 19, wherein the variant HSV TK polypeptide comprises an amino acid sequence having at least 80% amino acid sequence identity to a wild-type HSV TK, and comprises a substitution of one or more of L159, I160, F161, A168, and L169, based on the amino acid numbering of wild-type HSV TK amino acid sequence of SEQ ID NO: 25.

21. The RVV of claim 20, wherein the variant HSV TK polypeptide comprises an A168H substitution.

22. The RVV of claim 20, wherein the variant HSV TK polypeptide comprises an L159I substitution, an I160L substitution, an F161A substitution, an A168Y substitution, and an L169F substitution.

23. The RVV of claim 20, wherein the variant HSV TK polypeptide comprises an L159I substitution, an I160F substitution, an F161L substitution, an A168F substitution, and an L169M substitution.

24. The RW of claim 20, wherein the variant HSV TK polypeptide comprises the amino acid sequence of SEQ ID NO: 26, 27, or 28.

25. A composition comprising:
a) the RW of any one of claims 124; and
b) a pharmaceutically acceptable carrier.

26. A method of inducing oncolysis in an individual having a tumor, the method comprising administering to the individual an effective amount of the RVV of any one of claims 1-24, or the composition of claim 25.

27. The method of claim 26, wherein the tumor is a brain cancer tumor, a head and neck cancer tumor, an esophageal cancer tumor, a skin cancer tumor, a lung cancer tumor, a thymic cancer tumor, a stomach cancer tumor, a colon cancer tumor, a liver cancer tumor, an ovarian cancer tumor, a uterine cancer tumor, a bladder cancer tumor, a testicular cancer tumor, a rectal cancer tumor, a breast cancer tumor, or a pancreatic cancer tumor.

28. The method of claim 26, wherein the tumor is a colorectal adenocarcinoma, a non-small cell lung carcinoma, or a triple-negative breast cancer.

29. The method of any one of claims 26-28, wherein the tumor is recurrent.

30. The method of any one of claims 26-28, wherein the tumor is a primary tumor.

31. The method of any one of claims 26-28, wherein the tumor is metastatic.

32. The method of any one of claims 26-31, further comprising administering to the individual a second cancer therapy.

33. The method of claim 32, wherein the second cancer therapy is selected from chemotherapy, biological therapy, radiotherapy, immunotherapy, hormone therapy, antivascular therapy, cryotherapy, toxin therapy, oncolytic virus therapy, a cell therapy, and surgery.

34. The method of claim 32, wherein the second cancer therapy comprises an anti-PD1 antibody or an anti-PD-L1 antibody.

35. The method of any one of claims 26-34, wherein the individual is immunocompromised.

36. The method of any one of claims 26-35, wherein said administering of the RVV or the composition is intratumoral.

37. The method of any one of claims 26-35, wherein said administering of the RVV or the composition is peritumoral.

38. The method of any one of claims 26-35, wherein said administering of the RVV or the composition is intravenous.

39. The method of any one of claims 26-35, wherein said administering of the RVV or the composition is intra-arterial, intrabladder, or intrathecal.

40. The method of any one of claims 26-39, further comprising administering to the individual ganciclovir in an amount that is effective to reduce an adverse side effect of the vaccinia virus.

41. A replication-competent, recombinant oncolytic vaccinia virus (RVV), comprising, in its genome: (1) a nucleotide sequence encoding a variant interleukin-2 (IL-2v) polypeptide comprising SEQ ID NO: 9; (2) a nucleotide sequence encoding a heterologous TK polypeptide comprising SEQ ID NO:28; and (3) a K151E substitution in the A34R gene, wherein the RVV is a Copenhagen strain vaccinia virus and is vaccinia thymidine kinase deficient.

42. The RVV of claim 41, wherein the IL-2v polypeptide further comprises a signal peptide.

43. The RVV of claim 42, wherein the signal peptide comprises SEQ ID NO:22.

44. The RVV of any one of claims 8-24, wherein the nucleotide sequence encoding the variant IL-2v polypeptide comprises SEQ ID NO: 10.

45. The RVV of any one of claims 8-24, wherein the nucleotide sequence encoding the variant IL-2v polypeptide comprises SEQ ID NO: 12.

46. A composition, comprising: (i) the RVV of any one of claims 41 to 45 and (ii) a pharmaceutically acceptable carrier.
